(19) United States
(12) Patent Application Publication
(10) Pub. No.: US 2006/0029546 A1
(43) Pub. Date: Feb. 9, 2006

(54) Materials and methods for identifying anti-schizophrenic agents

(75) Inventors: Mark E. Gurney, Grand Rapids, MI (US); Sascha Haverfield-Gross, Chicago, IL (US); Thorkell Andressen, Reykjavik (IS); Maria Bjarnadottir, Reykjavik (IS)

Correspondence Address: MARSHALL, GERSTEIN & BORUN LLP, 233 S. WACKER DRIVE, SUITE 6300, SEARS TOWER, CHICAGO, IL, 60606, US

(73) Assignee: deCODE Genetics, Inc., Woodridge, IL (US)

(21) Appl. No.: 10/912,971

(22) Filed: Aug. 6, 2004

(51) Int. Cl. A61K 49/00; C12Q 1/68

(52) U.S. Cl. 424/9.1; 435/6

(57) ABSTRACT

The invention relates to materials and methods of identifying modulators of N-methyl-D-Aspartate (NMDA) receptor phosphorylation and function, including anti-schizophrenic agents that modulate NMDA receptor function. The methods of the invention utilize animal models of schizophrenia and cell-based assays to identify modulators of NMDA function. The invention further provides anti-schizophrenic agents identified by these methods. The invention relates to methods of treating schizophrenia using the modulators and anti-schizophrenic agents identified by the methods of the invention.

### FIELD OF INVENTION

The invention relates to materials and methods of identifying modulators of N-methyl-D-Aspartate (NMDA) receptor phosphorylation and function, including anti-schizophrenic agents that modulate NMDA receptor function. The methods of the invention utilize animal models of schizophrenia and cell-based assays. The invention further provides anti-schizophrenic agents identified by these methods. The invention relates to methods of treating schizophrenia using the modulators and anti-schizophrenic agents identified by the methods of the invention.

### BACKGROUND

Schizophrenia is a heritable, highly debilitating psychotic disorder that affects 0.5 to 1% of the general population. The illness is characterized by a variety of positive and negative signs and symptoms, as well as cognitive dysfunction that typically commence in early adulthood and often continue throughout life. The broad phenotypic presentation and a lack of complete disease concordance in monozygotic twins (˜50-60%) imply that a multitude of environmental and/or genetic factors might contribute to disease manifestation (Coyle et al., Ann. NY Acad. Sci. 1003: 318-27, 2003). With the discovery of a number of schizophrenia susceptibility genes, a molecular hypothesis has begun to emerge. Several of the recently described genes conferring susceptibility to the disease are believed to affect neuroplasticity, as well as glutamatergic neurotransmission (Harrison & Owen, Lancet 361: 417-9, 2003).

In a genome wide scan of schizophrenia families carried out in Iceland, a susceptibility gene was mapped to chromosome 8p21. Haplotype analysis identified Neuregulin 1 (NRG1) as a gene conferring susceptibility to schizophrenia (Stefannsson et al., Am. J. Hum. Genet., 72: 83-7, 2003). NRG1 as a schizophrenia disease gene has been replicated in multiple populations (Steffanson et al., Am. J. Hum. Genet. 72: 83-7, 2003; Williams et al., Mol. Psychiatry 8:485-7, 2003; Yang et al., Mol. Psychiatry 8:706-9, 2003). NRG1 is a polypeptide growth factor implicated in the modulation of neurotransmission in developing and adult synapses. Early studies focused on the neuromuscular junction, where NRG1 was identified as acetylcholine receptor-inducing activity (ARIA) factor (Jessell et al., Proc. Natl. Acad. Sci., 76: 5397-5401, 1979; Falls et al., J. Neurocytol., 32: 619-647, 2003).

Dopamine receptor antagonists, primarily D2 receptor selective antagonists, are used clinically for the control of the positive signs of schizophrenia, suggesting that the misregulation of dopamine neurotransmission contributes to disease pathophysiology (Freedman, N. Engl. J. Med., 349: 1738-1749, 2003). However, the dissociative anesthetics that block the NMDA receptor, such as phencyclidine (PCP) and ketamine, produce a schizophrenia-like disorder. Hence, a role for NMDA receptor hypofunction in the disease has also been suggested. (Reviewed in Konradi & Heckers Pharmacology & Therapeutics, 97: 153-197, 2003) Unlike manipulation of dopamine, for example by chronic exposure to amphetamines creating positive symptoms, exposure to dissociative anesthetics acutely reproduces the negative and cognitive signs of schizophrenia. NMDA receptors are ion channels: that function as coincidence detectors. They are simultaneously gated by voltage, as well as by two ligands, glutamate and glycine. Serine/threonine and tyrosine phosphorylation also strongly regulate NMDA receptor function (Yu et al., Science, 275: 674-678, 1997; Wang et al., Nature, 369: 233-235, 1994; Slater et al., Nat. Rev. Neurosci., 5: 317-328, 2004). Subtle misregulation of either membrane potential, ligand binding or tyrosine phosphorylation may therefore have profound effects on the probability and duration of NMDA channel opening, thus influencing behavior modulated by the NMDA receptor (Moghaddam, Neuron, 40: 881-884, 2003).

There is a need to identify new treatments for schizophrenia. To assist in the development of potential therapeutics and anti-schizophrenic agents, accurate and informative in vitro and in vivo assays for predicting and elucidating the effectiveness of potential treatments are needed.

### SUMMARY OF INVENTION

The present invention provides materials and methods that address one or more needs in the field.

For example, the invention includes methods of screening compounds to identify new biological modifiers that are useful as therapeutic agents for schizophrenia, or useful as lead compounds for developing therapeutic agents.

In one aspect, the invention is a method of identifying a modulator of N-methyl D-Aspartate (NMDA) receptor function comprising: (a) administering a test agent to a mammal harboring a genetic defect in a NRG1 signaling pathway, wherein the mammal has an NMDA receptor complex hypophosphorylation phenotype; (b) measuring tyrosine phosphorylation of NMDA receptor complex in the brain of the mammal after administering the test agent; and (c) identifying a modulator of NMDA receptor function from the measurement of NMDA receptor complex tyrosine phosphorylation. While the method can be practiced with single animals, the use of multiple animals is preferred, e.g., to permit statistical evaluation of the significance of results.

The method can be used to screen essentially any chemical, biochemical, or biological agent for its modulatory effects in the system in questions. Exemplary categories of agents, routes of administration, and suitable mammals are described in further detail below. Initial screens are preferably performed in non-human laboratory animals, such as mice and rats. Preclinical and clinical screening in higher mammals, such as porcine, primate and humans, also is contemplated.

NMDA receptors are heterodimeric complexes composed of a common NR1 subunit and at least one of four NR2 subunits (NR2A, NR2B, NR2C and NR2D) in an undetermined ratio. This “NMDA receptor complex” includes the NR1, NR2 and NR3 subunits and PSD95, as shown in FIG. 1. In addition, the NMDA receptor complex includes the ligands glycine and glutamate, which activate the receptor. NMDA receptors are activated upon NR1 subunit binding of glycine and the NR2 subunits binding glutamate with concurrent membrane depolarization. Upon activation, the receptor channel opens and Na⁺ and Ca²⁺ are transmitted through the 2+channel. The influx of Ca²⁺ activates kinases that phosphorylate cellular proteins within the synapse and in the nucleus, and this phosphorylation plays a role in either neuroplastic or cell death mechanisms.

The “NRG1 signaling pathway” is the cascade through which Neuregulin 1 (NRG1) signals through a tyrosine kinase transmembrane receptor that belong to the ErbB receptor family. The NRG1 signaling pathway is depicted in FIG. 1. The NRG1 polypeptide binds to ErbB2, ErbB3 and ErbB4, inducing homo- (ErbB2/2, ErbB4/4) and/or heterodimer (ErbB2/3, ErbB2/4 and ErbB3/4) formation. ErbB4 is the predominant receptor for NRG1 on neurons and is depicted in FIG. 1. NRG1 induced activation of ErbB4 leads to phosphorylation of tyrosine residues located in the cytoplasmic region of the receptor. NRG1 induced recruitment of ErbB4 binding proteins leads to activation of intracellular signaling cascades, resulting in biological responses. NRG1 signaling modulates NMDA receptor complex function through a mechanism involving activation of Fyn kinase activity and phosphorylation of Pyk2 on regulatory sites. Fyn, a non-receptor tyrosine kinase, is capable of binding to tyrosine-phosphorylated ErbB4, resulting in phosphorylation of Fyn Y420 (positive regulatory site) and increased Fyn kinase activity. Activation of the non-receptor kinases Fyn and Pyk2 modulates NMDA receptor function through phosphorylation of regulatory tyrosine residues on NR2 subunits. The term “NRG1 signaling pathway protein” refers to the signaling molecules involved in the NRG1 signaling pathway which include NRG1, ErbB4, Fyn, Pyk, DDEF2 and PSD95.

A “genetic defect in the NRG1 signaling pathway” refers to a mutation resulting in the elimination (knock out) or reduction (knock down) in expression of a NRG1 signaling pathway protein. A genetic defect in the NRG1 signal pathway also includes mutations that result in altered protein expression such as expression of activity-deficient proteins, constitutively active proteins or proteins with altered activity, such as a kinase that phosphorylates an abnormal target protein or regulatory site. Animals having a “NMDA receptor complex hypophosphorylation phenotype” are those which have a reduced level of phosphorylation of one or more proteins of the NMDA receptor complex, compared to a measurement of phosphorylation of the same protein in a wild type animal of the same species. Examples of mammals having a genetic defect in the NRG1 signaling pathway and an NMDA receptor complex hypophosphorylation phenotype include NRG1 heterozygous (NRG1^{+/-}) mice, ErbB4 heterozygous (ErbB4^{+/-) mice and Fyn homozygous knock out (Fyn}^{-/-}) mice. As demonstrated in Example 2 and depicted in FIG. 3, these mice have decreased phosphorylation of NMDA receptor subunit NR2B tyrosine residue 1472 (SEQ ID NO: 6) compared to wild type mice.

Step (b) of the method involves measuring tyrosine phosphorylation of NMDA receptor complex in the brain of the mammal after administering the test agent. A variety of laboratory methods exist for measuring tyrosine phosphorylation such as immunoassays using antibodies specific for phosphorylated proteins or phophorylated amino acids or mass spectrometry. These methods are described in greater detail below. In some variations, tyrosine phosphorylation is imaged in intact brain tissue. In other variations, brain tissue is removed and homogenized, and tyrosine phosphorylation is measured in the homogenate or fractions thereof.

The term “phosphorylation of NMDA receptor complex” refers to measuring phosphorylation of any protein within the NMDA receptor complex including the NMDA receptor subunits NR1, NR2 and NR3. In some variations, the measuring tyrosine phosphorylation of the NMDA receptor complex comprises measuring phosphorylation of a NMDA receptor. The term “NMDA receptor” refers to the channel made up of the NR1, NR2 and NR3 subunits. In some particular variations, the measuring tyrosine phosphorylation of the NMDA receptor complex comprises measuring phosphorylation of a NMDA receptor subunit NR2A. In some preferred variations, the measuring tyrosine phosphorylation of the NMDA receptor complex comprises measuring phosphorylation of a NMDA receptor subunit NR2B. In particular, measuring phosphorylation of a tyrosine residue that corresponds to residue 1472 of the NMDA receptor subunit NR2B amino acid sequence set forth in SEQ ID NO: 6 is a highly preferred variation. It will be appreciated that the tyrosine at position 1472 in SEQ ID NO: 6 may be located at a different position in allelic or species variants of NR2B. However, the corresponding position is easily identified using standard sequence alignment algorithms and confirmed using phosphotyrosine antibody screening.

Step (c) in the above-described method involves identifying a modulator of NMDA receptor function from the measurement of NMDA receptor complex tyrosine phosphorylation. A variety of analytical approaches can be used, both comparative and absolute. Comparative methods are preferred.

For example, in one variation, the identification is made by comparing results from a test animal with results in a genetically and phenotypically similar control animal, e.g., from the same inbred strain of mouse or rat. Thus, in one variation, step (c) comprises: comparing the measurement of NMDA receptor complex tyrosine phosphorylation in the brain of the mammal administered the test agent with a measurement of NMDA receptor complex tyrosine phosphorylation in the brain of a control mammal that did not receive the test agent, wherein the control mammal has the genetic defect and the NMDA receptor complex hypophosphorylation phenotype, and wherein a difference in NMDA receptor complex tyrosine phosphorylation measurements between the mammal administered the test agent and the control mammal identifies the test agent as a modulator of NMDA receptor function. In some preferred variations, a greater NMDA receptor complex tyrosine phosphorylation measurement in the mammal administered the test agent identifies the test agent as a positive modulator of NMDA receptor function. A positive modulator of NMDA receptor function increases ligand binding, increases channel opening and/or increases channel opening time. Positive modulation of NMDA receptor function may be indicated by an increase in the phosphorylation of the NMDA receptor complex or an increase in cellular calcium influx. A decrease in the level of tyrosine phosphorylation relative to a control, indicates that the test agent is an agent that negatively modulates NMDA receptor activity. Negative modulation of the NMDA receptor reduces NMDA receptor function e.g. by decreasing ligand binding, decreasing channel opening probability and/or decreasing channel open time.

In other variations, instead of comparing to a control animal of the same type, the comparison is made to a control animal have a “normal” phenotype. For example, step (c) optionally comprises: comparing the measurement of NMDA receptor complex tyrosine phosphorylation in the brain of the mammal administered the test agent with a measurement of NMDA receptor complex tyrosine phosphorylation in the brain of a control mammal free of the genetic defect and having a wild type NMDA receptor complex phosphorylation phenotype, wherein similar NMDA receptor complex tyrosine phosphorylation measurements in the mammal harboring the genetic defect and the control mammal identifies the test agent as a positive modulator of NMDA receptor function. A mammal having a “wild type NMDA receptor complex phosphorylation phenotype” is an animal that has a normal level of phosphorylation of the NMDA receptor complex or a level of phosphorylation of the NMDA receptor complex that naturally occurs in the mammal of that species. Preferably, the mammal having the wild type NMDA receptor phosphorylation phenotype may be free of neuronal defects and/or will not exhibit a behavioral symptom of schizophrenia.

In some variations of the above-described method, the administering step comprises administering two or more concentrations of the test agent to two or more of the mammals, wherein dose-dependent differences in NMDA receptor complex tyrosine phosphorylation measurements from in the mammals identifies the test agent as a modulator of NMDA receptor function.

In other variations, step (c) optionally comprises: comparing the measurement of NMDA receptor complex tyrosine phosphorylation in the brain of the mammal administered the test agent with a measurement of NMDA receptor complex tyrosine phosphorylation in brain of a control mammal that received an agent known to increase NMDA receptor complex tyrosine phosphorylation, wherein the control mammal has the genetic defect in a NRG1 signaling pathway and the NMDA receptor complex hypophosphorylation phenotype, and wherein similar NMDA receptor complex tyrosine phosphorylation measurements between the mammal administered the test agent and the control mammal identifies the test agent as a positive modulator of NMDA receptor function. An example of an agent known to increase NMDA receptor complex tyrosine phosphorylation is clozapine.

As another aspect, the invention includes methods of screening compounds to identify modulators that are useful as therapeutic agents for schizophrenia, or useful as lead compounds wherein the compounds are administered to an animal having a chemically-induced NMDA receptor hypophosphorylation phenotype.

For example, the invention is a method of identifying a modulator of N-methyl D-Aspartate (NMDA) receptor function comprising: (a)(1) administering an agent that reduces phosphorylation of an NMDA receptor complex to a mammal, in an amount effective to induce a NMDA receptor complex hypophosphorylated phenotype in a mammal and administering a test agent to the mammal; (a)(2) administering a test agent to the mammal, (b) measuring tyrosine phosphorylation of NMDA receptor complex in the brain of the mammal administered the test agent: (c) and identifying a modulator of NMDA receptor function from the measurement of tyrosine phosphorylation. While the method can be practiced with single animals, the use of multiple animals is preferred, e.g., to permit statistical evaluation of the significance of results.

The method can be used to screen essentially any chemical, biochemical, or biological agent for its modulatory effects in the system in questions. Exemplary categories of agents, routes of administration, and suitable mammals are described in further in detail. Initial screens are preferably performed in non-human laboratory animals, such as mice and rats. Preclinical and clinical screening in higher mammals, such as porcine, primate and humans, also is contemplated.

An agent that reduces phosphorylation of an NMDA receptor complex is any agent that reduces tyrosine or serine/threonine phosphorylation of the NMDA receptor complex. These agents are also referred to herein as “phosphorylation-reducing agents.” Examples of an agent that reduce phosphorylation of the NMDA receptor complex include phencyclidine (PCP), ketamine and amphetamine. An “agent-induced NMDA receptor complex hypophosphorylation phenotype” is a chemically induced reduction of the level of phosphorylation of one or more proteins of the NMDA receptor complex, compared to a measurement of phosphorylation of the same protein in a wild type animal of the same species.

Step (b) of the method involves measuring tyrosine phosphorylation of NMDA receptor complex in the brain of the mammal after administering the test agent. A variety of laboratory methods exist for measuring tyrosine phosphorylation such as immunoassays using antibodies specific for phosphorylated proteins or phophorylated amino acids or mass spectrometry. These methods are described in greater detail below. In some variations, tyrosine phosphorylation is imaged in intact brain tissue. In other variations, brain tissue is removed and homogenized, and tyrosine phosphorylation is measured in the homogenate or fractions thereof.

In some embodiments, the measuring tyrosine phosphorylation of the NMDA receptor complex comprises measuring phosphorylation of a NMDA receptor, in which the term “NMDA receptor” refers to a channel made up of NR1, NR2 and NR3 subunits. In some particular variations, the measuring tyrosine phosphorylation of the NMDA receptor complex comprises measuring phosphorylation of a NMDA receptor subunit NR2A. In some preferred variations, the measuring tyrosine phosphorylation of the NMDA receptor complex comprises measuring phosphorylation of a NMDA receptor subunit NR2B. In particular, measuring phosphorylation of a tyrosine residue that corresponds to residue 1472 of the NMDA receptor subunit NR2B amino acid sequence set forth in SEQ ID NO: 6 is a highly preferred variation. It will be appreciated that the tyrosine at position 1472 in SEQ ID NO: 6 may be located at a different position in allelic or species variants of NR2B. However, the corresponding position is easily identified using standard sequence alignment algorithms and confirmed using phosphotyrosine antibody screening.

Step (c) in the above-described method involves identifying a modulator of NMDA receptor function from the measurement of NMDA receptor complex tyrosine phosphorylation. A variety of analytical approaches can be used, both comparative and absolute. Comparative methods are preferred.

For example, in one variation, the identification is made by comparing results from a test animal with results in a genetically and phenotypically similar control animal, e.g., from the same inbred strain of mouse or rat. Thus, in one variation, step (c) comprises: comparing the measurement of NMDA receptor complex tyrosine phosphorylation in the brain of the mammal administered the test agent with a measurement of NMDA receptor complex tyrosine phosphorylation in the brain of a control mammal that did not receive the test agent, wherein the control mammal has the agent-induced NMDA receptor complex hypophosphrylated phenotype, and wherein a difference in NMDA receptor complex tyrosine phosphorylation measurements between the mammal administered the test agent and the control mammal identifies the test agent as a modulator of NMDA receptor function. In some preferred variations, a greater tyrosine phosphorylation measurement in the mammal administered the test agent identifies the test agent as a positive modulator of NMDA receptor function.

In other variations, instead of comparing to a control animal of the same type, the comparison is made a control mammal free of the agent-induced phosphorylation phenotype. For example, step (c) optionally comprises: comparing the measurement of NMDA receptor complex tyrosine phosphorylation in the brain of the mammal administered the test agent with a measurement of NMDA receptor complex tyrosine phosphorylation in the brain of a control mammal free of the agent-induced NMDA receptor complex hypophosphorylated phenotype and having a wild type NMDA receptor complex phosphorylation phenotype, wherein similar NMDA receptor complex tyrosine phosphorylation measurements in the mammal receiving the phosphorylation-reducing agent and the control mammal identifies the test agent as a positive modulator of NMDA receptor function. A mammal “free of the agent-induced NMDA receptor complex hypophopshorylation phenotype” is a mammal that has not been administered the phosphorylation-reducing agent. A mammal having a “wild type NMDA receptor complex phosphorylation phenotype” is an animal that has a normal level of NMDA receptor complex phosphorylation or the level of phosphorylation of the NMDA receptor complex that naturally occurs in an animal of that species.

In some variations of the above-described method, the administering step comprises administering two or more concentrations of the test agent to two or more of the mammals, wherein dose-dependent differences in NMDA receptor complex tyrosine phosphorylation measurements from in the mammals identifies the test agent as a modulator of NMDA receptor function.

As another aspect, the invention includes methods of screening compounds to identify modulators that are useful as therapeutic agents for schizophrenia, or useful as lead compounds wherein the compounds are administered to an animal exhibiting a behavioral symptom of schizophrenia.

In one aspect, the invention is a method of identifying modulators of N-methyl-D-aspartate (NMDA) receptor function comprising: (a) administering a test agent to a mammal exhibiting a behavioral symptom of schizophrenia, wherein the mammal has a NRG1 signaling pathway protein hypophosphorylation phenotype, (b) measuring tyrosine phosphorylation of a NRG1 signaling pathway protein in the brain of the mammal after administering the test agent; and (c) identifying a modulator of NMDA receptor function from the measurement of tyrosine phosphorylation of the NRG1 signaling pathway protein. While the method can be practiced with single animals, the use of multiple animals is preferred, e.g., to permit statistical evaluation of the significance of results.

The method can be used to screen essentially any chemical, biochemical, or biological agent for its modulatory effects in the system in questions. Exemplary categories of agents, routes of administration, and suitable mammals are described in further detail below. Initial screens are preferably performed in non-human laboratory animals, such as mice and rats. Preclinical and clinical screening in higher mammals, such as porcine, primate and humans, also is contemplated.

Behavioral symptoms of schizophrenia refer to behavioral traits in an animal that mimics a clinical aspect of schizophrenia in humans. Examples of the behavioral symptoms of schizophrenia include hyperactivity, decreased social interaction, defective memory function, defective cognitive function, decreased prepulse inhibition and combinations thereof. Described below are methods of measuring the behavioral symptoms of schizophrenia. Examples of mammals exhibiting behavioral symptom of schizophrenia, wherein the mammal has a NRG1 signaling pathway protein hypophosphorylation phenotype include NRG1^{+/-} mice and ErbB4^{+/-} mice. A “NRG1 signaling pathway protein hypophosphorylated phenotype” refers to an animal which has a reduced level of phosphorylation of one or more protein components of the NRG1 signaling pathway, compared to measurement of phosphorylation of the same protein in a wild type animal of the same species.

Step (b) of the method involves measuring tyrosine phosphorylation of a NRG1 signaling pathway protein of the mammal after administering the test agent. A “NRG1 signaling pathway protein” is a signaling molecule involved in the NRG1 signaling pathway. In some variations, measuring tyrosine phosphorylation of the NRG1 signaling proteins comprises measuring phosphorylation of ErbB4, Fyn or Pyk2. A variety of laboratory methods exist for measuring tyrosine phosphorylation such as immunoassays using antibodies specific for phosphorylated proteins or phophorylated amino acids or mass spectrometry. These methods are described in greater detail below. In some variations, tyrosine phosphorylation is imaged in intact brain tissue. In other variations, brain tissue is removed and homogenized, and tyrosine phosphorylation is measured in the homogenate or fractions thereof.

Step (c) in the above-described method involves identifying a modulator of NMDA receptor function from the measurement of tyrosine phosphorylation of the NRG1 signaling pathway protein. A variety of analytical approaches can be used, both comparative and absolute. Comparative methods are preferred.

For example, in one variation, the identification is made by comparing results from a test animal with results in a genetically and phenotypically similar control animal, e.g., from the same inbred strain of mouse or rat. Thus, in one variation, step (c) comprises: comparing the measurement of tyrosine phosphorylation of the NRG1 signaling pathway protein in the brain of the mammal administered the test agent with the measurement of tyrosine phosphorylation of the NRG1 signaling pathway protein in the brain of a control mammal not administered the test agent, wherein the control mammal exhibits the same behavioral symptom, and wherein a test agent that increases tyrosine phosphorylation of a positive regulatory site or an activation site of a NRG1 signaling pathway protein in the mammal administered the test agent is identified as a positive modulator of NMDA receptor function. NRG1 regulatory sites are residues that upon phosphorylation either activate (positive regulatory sites) or inhibit (negative regulatory sites) the signaling activity of the protein.

In other variations, instead of comparing to a control animal of the same type, the comparison is made to a control mammal having a “normal” phenotype. For example, step (c) optionally comprises: comparing the measurement of tyrosine phosphorylation of the NRG1 signaling pathway protein in the brain of the mammal administered the test agent with a measurement of tyrosine phosphorylation of the NRG1 signaling pathway protein in the brain of a wild type mammal that does not exhibit the symptom, wherein a test agent that increases the tyrosine phosphorylation of a positive regulatory site of a NRG1 signaling pathway protein in the mammal having the NRG1 signaling pathway protein hypophosphorylation phenotype to a level similar to the level of tyrosine phosphorylation of the NRG1 signaling pathway protein in the brain of the wild type mammal is identified to be a positive modulator of NMDA receptor function. A wild type mammal is a mammal that does not exhibit a behavioral symptom of schizophrenia. Preferably, the wild type mammal may be free of neuronal defects, mutations in the NRG1 signaling pathway and does not exhibit a NMDA receptor hypophosphorylation phenotype.

In another aspect, the invention is a method of identifying modulators of N-methyl-D-aspartate (NMDA) receptor function comprising: (a) administering a test agent to a mammal exhibiting a behavioral symptom of schizophrenia, wherein the mammal has a NMDA receptor complex hypophosphorylation phenotype, (b) measuring tyrosine phosphorylation of a NMDA receptor complex in the brain of the mammal after administering the test agent and (c) identifying a modulator of NMDA receptor function from the measurement of tyrosine phosphorylation of the NMDA receptor complex. While the method can be practiced with single animals, the use of multiple animals is preferred, e.g., to permit statistical evaluation of the significance of results.

The method can be used to screen essentially any chemical, biochemical, or biological agent for its modulatory effects in the system in questions. Exemplary categories of agents, routes of administration, and suitable mammals are described in further detail below. Initial screens are preferably performed in non-human laboratory animals, such as mice and rats. Preclinical and clinical screening in higher mammals, such as porcine, primate and humans, also is contemplated.

Behavioral symptoms of schizophrenia refer to behavioral traits in an animal that mimics a clinical aspect of schizophrenia in humans. Examples of the behavioral symptoms of schizophrenia include hyperactivity, decreased social interaction, defective memory function, defective cognitive function and decreased prepulse inhibition and combinations thereof. Described below are methods of measuring the behavioral symptoms of schizophrenia. Examples of mammals exhibiting a behavioral symptom of schizophrenia, wherein the mammal has a NMDA receptor complex hypophosphorylation phenotype include NRG1^{+/-} mice and ErbB4^{+/-}. See FIG. 3.

Step (b) of the method involves measuring tyrosine phosphorylation of a NMDA receptor complex in the brain of the mammal after administering the test agent. The methods may comprise measuring the tyrosine phosphorylation of the NMDA receptor. The invention also provides for measuring tyrosine phosphorylation of the NMDA receptor subunit NR2A or NR2B. Preferably, the method comprises measuring the phosphorylation of tyrosine residue 1472 of the NMDA receptor subunit NR2B having the amino acid sequence of SEQ ID NO: 6. A variety of laboratory methods exist for measuring tyrosine phosphorylation such as immunoassays using antibodies specific for phosphorylated proteins or phophorylated amino acids or mass spectrometry. These methods are described in greater detail below. In some variations, tyrosine phosphorylation is imaged in intact brain tissue. In other variations, brain tissue is removed and homogenized, and tyrosine phosphorylation is measured in the homogenate or fractions thereof.

Step (c) in the above-described method involves identifying a modulator of NMDA receptor function from the measurement of NMDA receptor complex tyrosine phosphorylation. A variety of analytical approaches can be used, both comparative and absolute. Comparative methods are preferred.

For example, in one variation, the identification is made my comparing results in a genetically and phenotypically similar control animal, e.g., from the same inbred strain of mouse or rat. Thus, in one variation, step (c) comprises: comparing the measurement of tyrosine phosphorylation of the NMDA receptor complex in the brain of the mammal administered the test agent with a measurement of tyrosine phosphorylation of the NMDA receptor complex in the brain of a control mammal not administered the test agent, wherein the control mammal exhibits the same behavioral symptom, and wherein a test agent that increases tyrosine phosphorylation in the mammal administered the test agent is identified as a positive modulator of NMDA receptor function.

In other variations, instead of comparing to a control animal of the same type, the comparison is made to a control animal having a “normal” phenotype. For example, step (c) optionally comprises: comparing the measurement of tyrosine phosphorylation of the NMDA receptor complex in the brain of the mammal administered the test agent with a measurement of tyrosine phosphorylation of the NMDA receptor complex in the brain of a wild type mammal that does not exhibit the symptom, wherein a test agent that increases the tyrosine phosphorylation in the mammal administered the test agent to a level similar to the level of tyrosine phosphorylation in the brain of the wild type mammal is identified to be a positive modulator of NMDA receptor function. A wild type mammal is a mammal that does not exhibit a behavioral symptom of schizophrenia. Preferably, the wild type mammal may be free of neuronal defects, mutations in the NRG1 signaling pathway and/or does not exhibit a NMDA receptor hypophosphorylation phenotype.

Any of the above-described methods of the invention may be carried out using mammals to identify modulators of NMDA receptor function, including non-human primates and rodents. For example, rodents useful for the invention include heterozygous NRG1 (NRG1^{+/-}) mice such as those generated by Harvey et al. (described in Steffanson et al., Am. J. Hum. Gen. 72: 83-87, 2003) and Erickson et al., (Development 24(24): 4999-5011, 1997), conditional NRG1 knock out mice, homozygous ErbB4 (ErbB4^{-/-}) mice such as the conditional ErbB4 knock mice generated by Golub et al. (Behav. Brain Res. 153(1): 159-170, 2004) and heterozygous ErbB4 (ErbB4^{+/-}) mice such as those generated by Gassmann et al. (Nature 378(6555): 390-394, 1995), homozygous Fyn (Fyn^{-/-}) mice such as those generated by Grant et al. (Science 258: 1903-1910, 1992), heterozygous Fyn (Fyn^{+/-}) mice, NR1 knock down mice such as those generated by Mohn et al. (Cell 98: 427-436, 1999) and heterozygous or knock down NR2A or NR2B mice. The invention also contemplates using rodents that have a genetic defect in any of the NRG1 signaling components including NRG1, ErbB4, Fyn or Pyk2. In addition, the methods of the invention contemplate using a mammal that is a rodent that exhibits at least one behavioral symptom of schizophrenia such as hyperactivity or increased psychomotor activity, stereotypic activity, decreased or impaired social activity, social withdrawal, impaired cognitive functions, impaired sensorimotor gating, impaired pre-pulse inhibition (PPI) and attentional defects Example of a mammal exhibiting at least one of these behavioral symptoms of schizophrenia include NMDA receptor subunit NR1^{-/-} mice, Reeler mice, DiGeorge mice, proline-dehydrogenase mutant mice and 5HT-2A receptor/mice.

The above-described methods of identifying modulators of NMDA receptor function comprise administering a test agent to a mammal. The invention contemplates formulating the test agent in a pharmaceutically acceptable carrier or a physiological acceptable carrier such as water, saline, salt solutions, buffered saline, alcohols, glycerol, ethanol, gum arabic, vegetable oils, benzyl alcohols, polyethylene glycols, gelatin, carbohydrates, dextrose, magnesium stearate, talc, silicic acid, viscous paraffin, perfume oil, fatty esters, hydroxymethylcellulose, polyvinyl pyroline or a combination thereof. The invention contemplates administering the test agent a route such as intradermal administration, intramuscular administration, intraperitoneal administration, intraocular administration, subcutaneous administration, topical administration, oral administration or intranasal administration. In a variation, any of the above-described methods may further comprising steps of monitoring the mammal that receives the test agent for behavioral symptoms of schizophrenia.

In another aspect, any of the above-described methods may optionally further comprise: (d) formulating a composition comprising a pharmaceutically acceptable carrier and a test agent identified as a modulator of NMDA receptor function in step (c) as a modulator of NMDA receptor function; (e) administering the composition to a mammal that exhibits at least one behavioral symptom of schizophrenia; and (f) monitoring the mammal for a decrease in the behavioral symptoms of schizophrenia.

As another aspect, the invention includes methods of screening compounds to palliate schizophrenia or useful as lead compounds using cell-based screening assays.

In one aspect, the invention is a method of screening for a candidate compound to palliate schizophrenia comprising: (a) contacting a test agent to a cell that expresses a NMDA receptor subunit NR2 and (b) measuring tyrosine phosphorylation of the NR2 subunit of the NMDA receptor of the cell in the presence and absence of the test agent, wherein an increase in tyrosine phosphorylation of the NMDA receptor NR2 subunit identifies an agent as a candidate compound to palliate schizophrenia. In some variations, the measuring comprises measuring tyrosine phosphorylation of NMDA receptor subunit NR2A or NR2B. In some preferred variations, the measuring comprises measuring phosphorylation of a tyrosine corresponding to the tyrosine at position 1472 of the NMDA receptor subunit NR2B having the amino acid sequence of SEQ ID NO: 6. It will be appreciated that the tyrosine at position 1472 in SEQ ID NO: 6 may be located at a different position in allelic or species variants of NR2B. However, the corresponding position is easily identified using standard sequence alignment algorithms and confirmed using phosphotyrosine antibody screening. Compounds that palliate schizophrenia are those agents that lessen or abate the symptoms of schizophrenia. Examples of behavioral symptoms of schizophrenia exhibited in humans include psychotic symptoms, stereotypic behavior, vulnerability to stress, informational processing defects, attentional defects, cognitive defects and social withdrawal.

The method can be used to screen essentially any chemical, biochemical, or biological agent for its modulatory effects in the system in questions. Exemplary categories of agents, routes of administration, and suitable mammals are described in further detail below. Initial screens are preferably performed in non-human laboratory animals, such as mice and rats. Preclinical and clinical screening in higher mammals, such as porcine, primate and humans, also is contemplated.

In variations, the methods comprise contacting a test agent to a cell that is recombinantly modified to express increased amounts of NMDA receptor subunit NR2. In some variations, the methods comprise contacting a test agent to a cell that further expresses a NRG1 signaling pathway protein, such as the NRG1 signaling pathway proteins ErbB4, Fyn or Pyk2. The above-identified methods may comprise a mammalian cell. Preferably, the methods comprise a neuron or a cell from a neuronal cell line such as BE(2)-M17 (ECACC No. 95011816) or SH-SY5Y (ATCC Accession No. CRL-2266).

In other variations, the above-described method of screening for a candidate compound to palliate schizophrenia may further comprise: (d) formulating a composition comprising a pharmaceutically acceptable carrier and a test agent identified in step (c) as a modulator of NMDA receptor function; administering the composition to a mammal that exhibits a behavioral symptom of schizophrenia; and (f) and monitoring the mammal for a decreased in the behavioral symptom of schizophrenia.

In another aspect, the invention is a method of identifying modulators of NMDA function comprising: (a) contacting a test agent to a cell that expresses a NMDA receptor subunit NR2 and the NRG1 signaling pathway proteins ErbB4 and Fyn, and (b) measuring tyrosine phosphorylation of a NRG1 signaling pathway protein in the presence and absence of the test agent, wherein an increase in tyrosine phosphorylation of a positive regulatory site or an activation site of a NRG1 signaling pathway protein identifies an agent that positively modulates NMDA receptor function; and a decrease in tyrosine phosphorylation of a negative regulatory site of a NRG1 signaling pathway protein the NRG1 signaling pathway protein in the presence of the test agent identifies an agent that negatively modulates NMDA receptor function.

The method can be used to screen essentially any chemical, biochemical, or biological agent for its modulatory effects in the system in questions. Exemplary categories of agents, routes of administration, and suitable mammals are described in further detail below. Initial screens are preferably performed in non-human laboratory animals, such as mice and rats. Preclinical and clinical screening in higher mammals, such as porcine, primate and humans, also is contemplated.

In any of the above-identified cell-based methods, the invention contemplates contacting a test agent with a cell transfected with a polynucleotide that encodes at least one protein of the NRG1 signaling pathway. In some variations, the methods comprise contacting at test agent with a cell that endogenously expresses the NMDA receptor subunit NR2 and the NRG1 signaling pathway proteins ErbB4 and Fyn, such as the neuroblastoma cell lines BE(2)-M17 (ECACC No. 95011816) and SH-SY5Y (ATCC Accession No. CRL-2266). In a particular variation, the method comprises contacting a test agent to a cell transfected with a polynucleotide that encodes at least one of the following NRG1 signaling pathway proteins: (a) ErbB4 polypeptide having the amino acid sequence of SEQ ID NO: 12, (b) Fyn polypeptide having the sequence of SEQ ID NO: 14, or (c) Pyk2 polypeptide having the sequence of SEQ ID NO: 16. In another variation, the method comprises contacting a test agent to a cell tranfected with a polynucleotide encoding at least one of the following NMDA receptor subunit NR2 polypeptide: (a) NR2A polypeptide having an amino acid sequence of SEQ ID NO: 4 and (b) the NR2B polypeptide having the amino acid sequence of SEQ ID NO: 6.

In another aspect, the invention is a method of identifying a modulator of NMDA receptor function comprising: (a) culturing a cell in the presence and absence of a test agent, wherein the cell expresses a NR2 polypeptide and polypeptide components of a NRG1 signaling pathway, and wherein the cell exhibits a physiological response or a biochemical response to NRG1, and (b) measuring tyrosine phosphorylation of a NRG1 signaling pathway protein in the presence and absence of the test agent, wherein an increase in tyrosine phosphorylation of a positive regulatory site or an activation site of a NRG1 signaling pathway protein in the presence of the test agent identifies the agent as a positive modulator or NMDA receptor function and wherein a decrease in tyrosine phosphorylation of a negative regulatory sites of a NRG1 signaling pathway protein NRG1 signaling pathway component in the presence of the test agent identifies an agent that is a negative modulator of NMDA receptor function. A physiological response to NRG1 is a cellular activity in response to NRG1 signaling such as regulation of expression of NRG1 response genes, induction of acetylcholine receptors and induction of sodium channels, stimulation of cell differentiation, stimulation of cell proliferation or stimulation of stem cell maturation. A biochemical response to NRG1 is activation or phosphorylation of a protein within the NRG1 signaling cascade in response to NRG1. Examples of a biochemical response to NRG1 include those described in Example 1, such as dimerization of ErbB4 receptor, phosphorylation of ErbB4, phosphorylation of Fyn tyrosine 420, phosphorylation of Pyk2 or Fyn binding ErbB4.

The foregoing summary is not intended to define every aspect of the invention, and additional aspects are described in other sections, such as the Detailed Description. The entire document is intended to be related as a unified disclosure, and it should be understood that all combinations of features described herein are contemplated, even if the combination of features are not found together in the same sentence, or paragraph, or section of this document.

In addition to the foregoing, the invention includes, as an additional aspect, all embodiments of the invention narrower in scope in any way than the variations defined by specific paragraphs above. For example, certain aspects of the invention that are described as a genus, and it should be understood that every member of a genus is, individually, an aspect of the invention. Also, aspects described as a genus or selecting a member of a genus, should be understood to embrace combinations of two or more members of the genus. Although the applicant(s) invented the full scope of the invention described herein, the applicants do not intend to claim subject matter described in the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

### BRIEF DESCRIPTION OF FIGURES

FIG. 1 is a schematic depicting the NRG1 signaling pathway through which NRG1 modulates phosphorylation of the NMDA receptor subunit NR2.

FIG. 2 depicts NRG1-induced modulation of the NMDA receptor complex in human BE(2)-M 17 neuroblastoma cells differentiated in retinoic acid measured by Western blot analysis. As shown from top to bottom, “ErbB4” detects ErbB4 expression which was used as a control for protein loading. “ErbB4 PY” detects tyrosine phosphorylation of ErbB4 protein. “Fyn/Src PY420” detects phosphorylation of Fyn protein on tyrosine residue 420. “Pyk2 PY402” detects phosphorylation of Pyk2 protein on tyrosine residue 402. “NR2B PY1472” detects phosphorylation of NMDA receptor subunit NR2B on tyrosine residue 1472.

FIG. 3 depicts hypophosphorylation of NMDA receptor subunit NR2B in NRG^{+/-} and ErbB4^{+/-} mutant mice. Western blot analysis was carried out on hippocampal lysates from wild type mice (lane 1), ErbB4^{+/-} (lane 2) and NRG1^{+/-} (lane 3), and Fyn^{-/-} mutant mice (lane 4). As shown in Panel A, NMDA receptor subunit NR2B Tyr1472 (NR2B PY1472) was hypophosphorylated tyrosine residue 1472 of the NR2B subunit the in hippocampal lysates from ErbB4^{+/-} and NRG1^{+/-} mutant mice compared to age and sex matched wild-type C57B1/6 mice. Phosphorylation of tyrosine residue 420 of Fyn (Fyn/Src PY420) also was reduced in ErbB4^{+/-} and NRG1^{+/-} mutant mice. Hippocampal lysates from Fyn^{-/-} null mice served as a control for NR2B Tyr1472 hypophosphorylation. In Panel B, Clozapine reversed NR2B hypophosphorylation in NRG1^{+/-} mutant mice. NR2B Tyr1472 phosphorylation was increased 2.5-3 fold in NRG1^{+/-} mutant mice (n=3). The same dose of clozapine had no effect on NR2B Tyr1472 phosphorylation when administered to age and sex matched wild-type mice (n=2).

FIG. 4 depicts phosphorylation of the ErbB4 polypeptide (Panel A) and the NMDA receptor subunit NR2B (Panel B) on tyrosine residue 1472 (SEQ ID NO: 6) in the presence of a modulator of NMDA receptor function identified by the methods of the present invention.

### DETAILED DESCRIPTION

The invention provides methods of identifying molecules that modulate NMDA receptor function using animal models of schizophrenia or cells in culture which express the NMDA receptor complex and components of the NRG1 signaling pathway. Modulators that increase the phosphorylation of NMDA receptors are useful for the treatment of schizophrenia. Glutamate neurotransmission is known to play a role in schizophrenia. In particular, facilitation of the ionotrophic glutamate receptor N-methyl-D-aspartate (NMDA) receptor is contemplated to be therapeutically beneficial to schizophrenia patients. Thus, the present invention provides methods of identifying agents that will positively modulate NMDA receptor function as measured by tyrosine phosphorylation of the NMDA receptor.

Neuregulin 1

Neuregulin 1 (also called ARIA, GGF2 and heregulin) is a group of polypeptide factors that arise from alternative RNA splicing of a single gene (Fischbach & Rosen, Annu. Rev. Neurosci. 20:429-458, 1997; Orr-Urtreger, et al., Proc. Natl. Acad. Sci. USA 90:1746-1750, 1993; see also, Corfas et al., Neuron 14(1):103-15, 1995 and Meyer et al., Development 124(18):3575-86, 1997). The basic structure of neuregulin 1 (NRG1) includes a N-terminal region, an immunoglobulin (Ig) motif, a glycosylation-rich spacer domain, an EGF-like domain, and a cytoplasmic tail. See Fischbach & Rosen, Annu. Rev. Neurosci. 20:429-458, 1997; Loeb et al., Development 126(4):781-91, 1999; and Meyer et al., Development 124(18):3575-86, 1997.

NRG1 isoforms are expressed in many tissues, including the central nervous system (CNS), and these isoforms clearly have a developmental role as indicated by knockout mice displaying severe developmental anomalies in the heart and the nervous system (Gerlai et al. Behav. Brain Res. 109: 219-227, 2000; Liu et. al, Proc. Natl. Acad. Sci., 95: 13024, 1998). A NRG1 isoform originally discovered on the presynaptic membrane of the motor neuron (as ARIA), was found to be the factor inducing expression and localization of acetylcholine receptors (AChR) at the neuromuscular synapse. NRG1 isoforms influence gliogenesis and neuronal migration during development of the brain, while in the adult nervous system, NRG1 appears to have a marked impact on the expression and activation of several neurotransmitter receptors including NMDA glutamate receptors (Ibrahim et al., Am. J. Psychiatry, 157: 1811, 2000), possibly Ca²⁺ activated K⁺ channels (Chu et al., Neuron, 14: 329, 1995; Cameron et al., Proc. Natl. Acad. Sci. 98: 2832, 2001) and it facilitates neurotransmitter release from GABAergic interneurons.

The transmembrane forms of NRG1 are present within synaptic vesicles, including those containing glutamate. After exocytosis, NRG1 is in the presynaptic membrane where the ectodomain of NRG1 may be cleaved off. The ectodomain then migrates across the synaptic cleft and binds to and activates a member of the EGF receptor family on the postsynaptic membrane

Neuregulin and Schizophrenia

DNA polymorphisms in the NRG1 gene are an important genetic risk factor for susceptibility to schizophrenia. The NRG1 gene is contemplated to be associated with schizophrenia for many reasons, including its role in the expression of the N-methyl-D-aspartate (NMDA) receptor, in activation of AChR gene expression as well as activation of epidermal growth factor receptors and GABA(a) receptor subunits, and also its induction of components in a G-protein signaling cascade. Each of these activities of NRG1 is discussed briefly below.

NRG1 is involved in the expression of the NMDA receptor subunits (Mohn et al. Cell 98(4):427-36, 1999). NMDA receptors expressed in brain are made up of at least one NR1 subunit and a NR2 subunit (A, B, C and/or D). Genetically engineered mutant mice expressing only 5% of the normal number of NR1 subunits display schizophrenic features.

NRG1 is a potent activator of AChR gene expression. The neural signals proposed to induce the mRNA expression of acetylcholine receptors in muscle include neuregulin (NRG1). Neuregulin increases AChR expression by binding and activating ErbB receptor tyrosine kinases, including the recruitment of the SH2 domain protein SCH, and subsequently activating the Ras/Raf, MAPK cascade (Lindstrom, Mol. Neurobiol. 15(2):193-222, 1997). Pathogenic roles of AChRs are being discovered in many diseases involving mechanisms ranging from mutations, to autoimmune responses, and involving signs and symptoms ranging from muscle weakness to epilepsy, to neurodegenerative disease, to psychiatric disease, to nicotine addiction (id.). A high proportion of schizophrenics are intense tobacco users. It has been suggested that they may be attempting to self-medicate. Mutation in the NRG1 gene may alter the expression of the AChR gene and, through that mechanism cause the disease.

One important function of NRG1 is interaction with the ErbB family of receptors to assist in regulating cell growth and differentiation. For example, neuregulin activates the epidermal growth factor receptors ErbB3 and ErbB4 (Zhu et al., EMBO J. 14(23):5842-8, 1995; Komblum, et al., Dev. Neurosci. 22(1-2):15-24, 2000). Expression of NRG1 and the ErbB receptors in the developing nervous system is indicative of their role in neural development, including the regulation of cell fate specification, proliferation and survival in the neural crest lineage. Recent evidence indicates that ErbB3 and ErbB4 play an important role in the development of the CNS. Some theories on the causes of schizophrenia postulate that the disease is caused by defective brain development and there are studies that support the presence of neurodevelopmental abnormalities in schizophrenia (Kornblum et al., Dev. Neurosci. 22(1-2):16-24, 2000).

NRG1 induces expression of the GABA(A) receptor beta2 subunit. This increase in subunit expression is paralleled by an increase in functional GABA(A) receptors (Rieff et al., J. Neurosci. 19(24):10757-66, 1999). One hypothesis is that the pathophysiology of schizophrenia may be associated with a dysfunction in GABA transmission in the human prefrontal cortex. Dysfunction of the dorsolateral prefrontal cortex appears to be a central feature of the pathophysiology of schizophrenia, and this dysfunction may be related to alterations in gamma aminobutyric acid (GABA) neurotransmission (id.).

Genetic evidence suggests a linkage of schizophrenia to chromosome 8p in Icelandic families. This observation is supported by evidence of linkage in four other populations. The genetic relevance in humans is further supported by highly significant association of overlapping DNA haplotypes, that contain only one gene within the overlap, namely NRG1. The population attributed risk for the identified core haplotype is 16%, which is a substantial contribution to the public health burden.

In addition, as described in detail below, mice heterozygotic for each of two mutations in NRG1 gene and one mutation in a receptor for NRG1 (ErbB4) display behavioral alterations that overlaps with mouse models for schizophrenia, and this behavior is reversed with clozapine in a NRG1 mutant line. The number of NMDA receptors in the NRG1 heterozygotic mice were reduced, which agrees with observations made on brains from human schizophrenia patients. These data indicate that variants of the NRG1 gene contribute to the pathogenesis of schizophrenia in some patients, probably through a decrease in NRG1 signaling. The overlap in behavioral phenotype between the NRG1 and ErbB4 heterozygotic mice, and the lack of a similar behavioral phenotype in ErbB2 or ErbB3 mice (Gerlai et al., Behav. Brain Res. 109: 219-227, 2000), suggest that the defect in these mice is primarily neuronal. This is evidence that NRG1 plays a significant role in the pathogenesis of schizophrenia. Modulators of NRG1 signaling are contemplated as therapeutics for schizophrenia, in particular those modulators which positively modulate NRG1 signaling and thereby increase phosphorylation of the NMDA receptor complex.

Neuregulin Signaling

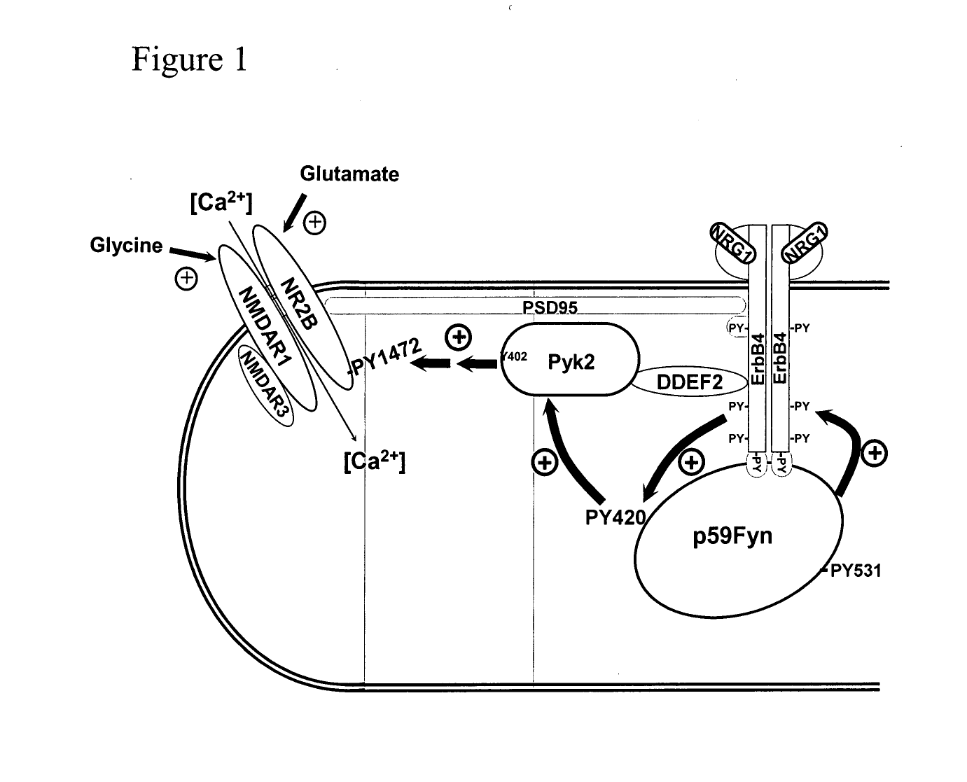

The polypeptide growth factor Neuregulin 1 (NRG1) signals through a tyrosine kinase transmembrane receptor family known as the ErbB receptors. The four different receptors (ErbB1-4) that belong to this family all have high protein sequence homology. The NRG1 polypeptide binds to ErbB2, ErbB3 and ErbB4, inducing homo- (ErbB2/2, ErbB4/4) and/or heterodimer (ErbB2/3, ErbB2/4 and ErbB3/4) formation. ErbB4 is the predominant receptor for NRG1 on neurons and is depicted in FIG. 1. NRG1 induced activation of ErbB4 leads to phosphorylation of tyrosine residues located in the cytoplasmic region of the receptor. Examples of tyrosine residues that are phosphorylated upon NRG1 binding include Y1056, Y1188 and Y1242. These tyrosine residues have been identified as docking sites for the SH2 domain containing proteins Shc (Y1188 and Y1242) and P13 kinase (Y1056). NRG1 induced recruitment of these and other ErbB4 binding proteins leads to activation of intracellular signaling cascades, resulting in biological responses.

The experiments described in Example 1, demonstrate that NRG1 signaling modulates NMDA receptor complex function through a mechanism involving activation of Fyn kinase activity and phosphorylation of Pyk2 on regulatory sites. Fyn, a non-receptor tyrosine kinase, is capable of binding to tyrosine-phosphorylated ErbB4, resulting in phosphorylation of Fyn Y420 (positive regulatory site) and increased Fyn kinase activity. Fyn Y420 is an example of a NRG1 signaling pathway protein regulatory site or activation site. In brain, ErbB4, Fyn, Pyk2 and PSD95 as well as the NMDA receptor are members of the postsynaptic density.

Activation of the non-receptor kinases Fyn and Pyk2 modulates NMDA receptor function through phosphorylation of regulatory tyrosine residues on NR2 subunits. NRG1 regulatory sites are residues that upon phosphorylation either activate (positive regulatory sites) or inhibit (negative regulatory sites) the signaling activity of the protein. For example, phosphorylation of a positive regulatory site, such as Fyn Y420, induces the NRG1 signaling cascade downstream of the phosphorylated protein. Alternatively, phosphorylation of a negative regulatory site, such as Fyn Y531 shuts down or deactivates the NRG1 signaling cascade downstream of the phosphorylated protein. Tyrosine phosphorylation of residue 1472 of the NMDA receptor subunit NR2B has been demonstrated in the brain. NMDA NR2 tyrosine phosphorylation by Fyn increases NMDA receptor channel open probability and mean open time.

The invention contemplates that NRG1 signaling, through activation of Fyn and Pyk2, will modulate NMDA receptor complex function and induce phosphorylation of any intracellular tyrosine residue of the NMDA receptor subunit NR2 or any intracellular serine or threonine residue of the NMDA receptor subunit NR1.

The signaling pathway described above and depicted in FIG. 1 is referred to herein as a “NRG1 signaling pathway” and the signaling molecules involved in the pathway are referred to herein as “NRG1 signaling pathway components” or “NRG1 signaling pathway proteins.” The NRG1 signaling pathway components are depicted in FIG. 1. The central NRG1 signaling components referred to herein are ErbB4, Fyn and Pyk2. The invention provides for methods of screening modulators of NMDA receptor function utilizing the NRG1 signaling pathway, including identifying modulators of the interactions of Fyn and Pyk2 with ErbB4 or NRG1. As schizophrenia correlates with hypophosphorylated NMDA receptors, agents that enhance NRG1 signaling, such as NRG1-induced activation of Fyn or Pyk2, are contemplated as therapeutics for schizophrenia.

NRG1 acts as a trophic factor for neurons and glia cells and regulates the expression of genes important for neuronal biology such as neurotransmitter receptors and voltage-gated ion channels. Both NRG1 and the ErbB receptors are widely expressed during development and in the adult. ErbB3 and ErbB4 are the major ErbB receptors in brain although low levels of ErbB2 expression is found in glia. Of these, ErbB4 is the principal receptor for NRG1 on neurons. It is most abundant in the cerebral cortex, slightly lower in the midbrain, and lowest in the cerebellum and brainstem. There is a good spatial correlation between expression of NRG1 and ErbB4 in the central nervous system and more importantly, the pattern of ErbB4 expression correlates well with the neuronal circuitry that has been implicated in schizophrenia. For example, in the cortex, ErbB4 is expressed by GABAergic interneurons, a subset of these appear to be primarily affected in schizophrenia. In the hippocampus, a region of the brain thought to be prominently involved in schizophrenia, the GABAergic interneurons also express ErbB4 and also express the NMDA receptor complex.

It appears that schizophrenia is caused by a defect in NRG1/ErbB4 signaling that leads to decreased neurotransmission function in the GABAergic interneurons and even perhaps to decreased numbers of the GABAergic interneurons; therefore, to treat schizophrenia (e.g., to correct the defect), an agent that potentiates NRG1 signalling or ErbB4 kinase activity can be used.

NMDA Receptor Complex

N-methyl-D-aspartate (NMDA) receptors are a subclass of ionotrophic glutamate receptors which play a role in synapse refinement, neuronal plasticity and excitotoxicity. NMDA receptors are heterodimeric complexes composed of a common NR1 subunit and at least one of four NR2 subunits (NR2A, NR2B, NR2C and NR2D) in an undetermined ratio. This “NMDA receptor complex” includes the NR1, NR2 and NR3 subunits and PSD95. In addition, the NMDA receptor complex includes glycine and glutamate which activate the receptor. See FIG. 1. The term “NMDA receptor” refers to the channel made up of the NR1, NR2 and NR3 subunits. NMDA receptors are activated upon NR1 subunit binding of glycine and the NR2 subunits binding glutamate with concurrent membrane depolarization. Upon activation, the receptor channel opens and Na⁺ and Ca²⁺ are transmitted through the channel. The influx of Ca²⁺ activates kinases that phosphorylate cellular proteins within the synapse and in the nucleus, and this phosphorylation play a role in either neuroplastic or cell death mechanisms such as apoptosis. It is known that the glutamate system is hypoactive in schizophrenia, and stimulation of glutamate receptors, such as NMDA receptors, is a therapeutic goal for schizophrenic patients. (Reviewed in Konradi & Heckers, Pharm Ther. 97: 153-179, 2003).

NMDA receptor activity is highly regulated by modulation of intracellular and extracellular sites including phosphorylation on tyrosine residues. In particular, NR2 subunits are tyrosine phosphorylated and this phosphorylation modulates the receptor properties. Nonreceptor kinases, Fyn and Pyk2, are known to phosphorylate NR2A and NR2B subunits. In particular, it has been reported that Tyr1472 of NR2B is the principle Fyn-mediated phosphorylation site (Nakazawa et al., J. Biol. Chem. 276: 693-699, 2001).

Assays for Evaluating NMDA Function

The invention provides methods of identifying modulators of NMDA function. One embodiment of the invention identifies modulators of NMDA function by measuring phosphorylation of the NMDA receptor complex. NMDA receptor function may be evaluated by determining the level of tyrosine phosphorylation of the NR2 subunit or determining the level of serine/threonine phosphorylation of the NR1 subunit. As demonstrated in Example 2, NRG1 signaling induces tyrosine phosphorylation of the NR2B subunit and Example 3 demonstrates that the NMDA receptors in NRG1 heterozygous mice and ErbB4 heterozygous mice have reduced phosphorylation of the NMDA receptor subunit NR2B.

Tyrosine phosphorylation may be detected with an antibody specific for phosphotyrosine residues. Examples of anti-phosphotyrosine antibodies include commercially available antibodies 4G10 (Upstate Biotechnology,) ab9329 and 13F9 (Abcab, Inc., Cambridge, Mass.), and commercially available antibodies PY20, PT66, P-Tyr-0.100 (PerkinElmer, Wellesley, Mass.). In an embodiment of the invention, the animal are administered a test agent and the hippocampus is dissected from the brain and hippocampal homogenates are prepared. The proteins of interest, such as an NMDA receptor subunit or a NRG1 signaling pathway protein, are immunoprecipitated from the homogenates using standard methods in the art and described herein. The invention also provides for cell-based assays that measure the tyrosine phosphorylation of the NMDA receptors of cells in culture. After contacting the cell with a test agent, the cells may be lysed and the protein of interest is immunoprecipitated from the cell lysate. The immunoprecipitated proteins from the brain homogenates and the cell lysates are purified on a SDS-PAGE gel and phosphorylation of the protein is detected by Western blot analysis with a phosphotyrosine antibody.

Further, antibodies that are specific for phosphoryated proteins such as phosphoryated NMDA receptor subunit NR2B, may be used in the methods of the invention. These antibodies can detect the phosphorylated proteins of interest such as anti-phospho-ErbB4, anti-Fyn/Src PY420 (Biosource, Camarillo, Calif.) and anti-NR2B-phospho-specific Tyr1472 (Calbiochem, San Diego, Calif.) by Western blot and thereby surpassing the immunoprecipitation step described above.

Imaging analysis may be used to quantify antibody binding on a Western blot in order to quantify the tyrosine phosphorylation of the protein of interest. Fluorescent and chemiluminescent labels such as ECL Western Blot detection system exemplified herein, may be used to quantity antibody binding. Densitometry may also be used to quantify the tyrosine phosphorylation.

An embodiment of the invention, measures the phosphorylation of a the NMDA receptor subunit. There are commercially available antibodies that specifically bind the NMDA receptor complex including those that are specific for phosphorylated NMDA receptor subunits which may be used to immunoprecipitate the NMDA receptor subunit or to detect the NMDA receptor subunit on a Western Blot. Examples of commercially available antibodies specific for NR1 is antibody IQ183 (Immunoquest, Ltd., Cleveland, UK). Examples of commercially available antibodies specific for NR2A are A-6473 (Molecular Probes, Eugene, Oreg.), IQ1884 (Immunoquest, Ltd., Cleveland, UK), 9061-005 (Signet, Dedham, Mass.). Examples of commercially available antibodies specific for NR2B are NRB2-Phospho-Specific Tyr1472 (Calbiochem, San Diego, Calif.), 9063-005 (Signet, Dedham, Mass.), NB300-182 (Novus Biologicals, Littleton, Colo.), A-6474 (Molecular Probes, Eugene, Oreg.) and IQ185 (Immunoquest, Ltd., Cleveland, UK). Examples of commercially available antibodies specific for NR2C and NR2D is IQ186 (Immunoquest, Ltd., Cleveland, UK).

Another embodiment of the invention measures the tyrosine phosphorylation of a NRG1 signaling pathway proteins such as ErbB4, Fyn, Pyk2. The NRG1 signaling pathway proteins may be immunoprecipitated with an antibody specific for the protein such as commercially available antibodies. Examples of commercially available antibodies specific for ErbB4 are sc-283 (Santa Cruz Biotechnology, Santa Cruz, Calif.), MAB 1131 (R&D Systems, Minneapolis, Minn.), HM1133 (Hypomatrix, Worcester, Mass.), RB9045 (NeoMarkers, Fremont, Calif.) and E5900 (Sigma, St. Louis, Mo.). Examples of commercially available antibodies specific for Fyn are Fyn/Src PY420 (Biosource, Camarillo, Calif.) and Fyn/Src PY531 (Biosource, Camarillo, Calif.) FynAB-1 (NeoMarkers, Fremont, Calif.) and FA1075 (Fusion Antibodies, Belfast, Ireland). Examples of commercial antibodies for Pyk2 are sc-9019 (Santa Cruz Biotechnology, Santa Cruz, Calif.) P3902 and P6614 (Sigma, St. Louis, Mo.). The invention may be carried out with any antibody that specifically binds the NMDA receptor subunit NR2 or an NRG1 signaling pathway protein and any antibody that specifically binds phosphorylated tyrosine residues. In a particular embodiment, the method comprises assessing NMDA receptor function by measuring the phosphorylation of tyrosine 1472 on the NR2B subunit.

The invention also contemplates immunoprecipitating the NR1 subunit and detecting phosphorylated serine and threonine residues with an antibody specific for phospho-serine/threonine residues. Examples of commercially available antibodies specific for phospho-serine and phospho-threonine residues are 12-362 (Upstate Biotechnology, Waltham, Mass.), S38820 (BD Transduction, San Jose, Calif.) and p34cdc2 (Biodesign, Saco, Me.).

Protein expression or protein post-translational modification levels may be measured by mass spectrometry using electrospray (ESI) or matrix-assisted laser desorption/ionization (MALDI) mass spectrometers. The concentration of a phosphorylated protein of interest (analyte concentration) may be measured with mass spectrometry data. When a known quantity of an internal standard is added to the analytical sample. Following extraction and sample preparation steps, the ratio of intensities or areas of the peaks recorded by the mass spectrometer can be used to calculate analyte concentration.

In an alternative approach, a proteolytic digestion of the target protein(s) and measuring surrogate peptide analyte(s) using mass spectrometry may be a more efficient method for measuring the level of phosphorylated protein. Peptide sequencing also allows for confirmation of analyte identity rather than evaluating a protein molecular weight alone. This approach relies on being able to prove complete proteolysis of the target protein or at least equivalent proteolysis of the target protein between samples, (which could be shown for example by Western blotting following the digestion step). For example, analysis of Tyr-1472 on NMDA receptor subunit NR2B, a tryptic digest of the protein would result in generation of the phosphopeptide AFNGSSNGHVY*EK (SEQ ID NO: 18) as well as some non-phosphorylated variant (Y* refers to the phosphorylated residue of the peptide). A sample preparation method which allows the detection of this phosphorylated peptide in the mass spectrometer is generated. Generally, immobilized metal affinity chromatography (IMAC) or immunoaffinity extraction are commonly used methods to extract phosphopeptides from complex peptide mixtures prior to ESI-MS or MALDI-MS. If the phosphorylated peptide cannot be detected but the non-phosphorylated peptide variant can be detected, an internal standard corresponding to the non-phosphorylated peptide could be synthesized. Following addition of the internal standard, the sample is split into two. The first portion of the sample is analyzed to quantify the amount of protein without phosphorylation. The remainder of the sample would be subjected to an additional proteolysis step, to remove any phosphorylation from the peptide, before determination. The difference between the level of peptide before and after dephosphorylation would represent the proportion of protein phosphorylated.

Another option to measure tyrosine phosphorylation with mass spectrometry applies a two-dimensional approach for relative quantification between samples. Similarly, this approach involves mass spectrometry analysis of surrogate peptide analytes following proteolytic digestion, but in this case isotopic differences between two whole protein or peptide pools are introduced before combining them. Isolation and separation of the corresponding surrogate peptide analytes of interest analytes, and comparing ion abundances for the “light” and “heavy” counterparts allows relative quantification of the protein in question in the mass spectrometer. In this approach, the samples may undergo metabolic isotope labeling or chemical derivatisation strategies. Metabolic labeling methods introduce isotope labels into proteome pools by growing cells on isotopically distinct culture media, for example, one culture grown on media containing the natural abundance of isotopes, the other grown on the same medium enriched in ¹⁵N. This can also be accomplished by growing cells in medium deficient of certain amino acids, and supplementing isotopically labeled amino or non-isotopically labeled amino acids for growth of the two cell variants. The metabolic labeling method however does not allow for relative quantification of proteins extracted from tissue. Chemical derivatisation of proteins or peptides post-extraction is more versatile. Various examples include the well documented isotope-coded affinity tag (ICAT) approach (Gyni et al., Nat, Biochem. 17:994-999, 1999), performing tryptic digestion in H₂O or H₂¹⁸O proteins in one pool are cleaved to generate peptides containing two ¹⁸O atoms at their C-termini) or methylesterification of carboxylic acid groups present on Asp and Glu residues, as well as the C-termini, with ²H₀- or ²H₃-methanolic-HCl. Saloman et al. (PNAS, 100(2), 443-448, 2003) demonstrates that this derivatisation method with IMAC and nanoLC-MS may be used to profile tyrosine phosphorylation in human cells.

In one embodiment, the level of phosphorylation of the NMDA receptors of the brain of the mammals administered the test agent are measured. The brains of the mammals administered the test agent may be obtained by killing the mammal, dissecting free the hippocampus and preparing homogenates of the hippocampal tissue. The hippocampal tissue is preferred for biochemical analysis of NMDA receptor complex phosphorylation. In another embodiment, the phosphorylation is detected by staining slices of the hippocampus using immunohistochemical detection methods. The invention also contemplates using portions of the brains obtained by biopsy from a viable mammal.

If the level of the NMDA receptor function in the presence of the test agent differs, preferably by an amount that is statistically significant, from the level of the activity in the absence of the test agent, then the test agent is an agent that modulates NMDA receptor function. An increase in the level of tyrosine phosphorylation relative to a control, indicates that the agent is an agent that positively modulates NMDA receptor activity. Positive modulation of the NMDA receptor enhance NMDA receptor function e.g. by increasing ligand binding, increasing channel opening probability and/or increasing channel open time. Positive modulation NMDA receptor function may be indicated by an increase in the phosphorylation of the NMDA receptor complex and/or an increase in cellular calcium influx. Positive modulators of NMDA receptor function include agonist that directly activate the NMDA receptor to invoke a greater response by the NMDA receptor. Positive modulators of NMDA receptor function also include agents that act indirectly to enhance NMDA receptor function.

Similarly, a decrease in the level of tyrosine phosphorylation relative to a control, indicates that the test agent is an agent that negatively modulates NMDA receptor activity. Negative modulation of the NMDA receptor reduces NMDA receptor function e.g. by decreasing ligand binding, decreasing channel opening probability and/or decreasing channel open time. Negative modulators of NMDA receptor function include antagonists that directly inhibit the NMDA receptor to invoke a reduced response by the NMDA receptor. Negative modulators of NMDA receptor function also include agents that act indirectly to reduce NMDA receptor function. For the methods of identifying modulators of NMDA function wherein the test agent is administered to an animal, the contemplated controls include animals of the same species not administered the test agent, preferably litter mates and wild type animals of the same species. For the cell-based assays for identifying modulators of NMDA function, the contemplated controls are cells of the same type or cell line that are not contacted with the test agent.

Serine/threonine and tyrosine phosphorylation are indicators of NMDA receptor activation and phosphorylation also strongly modulate NMDAR function (Yu et al., Science 275: 674-678, 1997; Wang & Salter Nature 369: 233-235, 1994; Slater & Kalia, Nat. Rev. Neurosci. 5: 317-328, 2004). For example, Src-family kinases has been shown to phosphorylate the NMDAR subunit NR2B on tyrosine 1472 (Tyr1472), thereby increasing NMDAR channel open time and channel open probability (Nakazawa et al., J. Biol. Chem. 276: 693-696, 2001; Yu et al., Science. 275: 674-678, 1997). This phosphorylation correspondingly facilitates NMDA receptor neurotransmission.

Activated NMDA receptors transmit calcium into the cell and the invention contemplates measuring calcium influx into a cell contacted with the test agent of the present invention. In one embodiment, electrophysiological techniques may be used to measure influx of calcium into a cell or in whole hippocampal slices. The concentration of intracellular calcium may be measured using intracellular electrodes, such as a calcium selective microelectrode. The calcium influx through the NMDA receptor may be measured using patch clamp techniques, which clamp the voltage across the receptor to maintain a fixed voltage. For example, a cell or a hippocampal slice is placed in a perfusion chamber, and the NMDA receptor field excitory postsynaptic potentials are recorded in the absence and presence of the test agent. For specificity to the NMDA receptor response, the GABA and AMPAR receptor responses may be blocked using selective inhibitors such as NBQX (AMPAR blocker) and picotoxin (GABA blocker), and the experiments may be carried out with a NMDA receptor antagonist such as DL-2-amino5-phosphonovaleric acid. (See Alvestad et al., J. Biol. Chem. 278: 11020-11025).

Calcium influx may also be monitored with fluorescent indicators that show a spectral response upon binding calcium. The use of fluorescent indicators allows calcium concentrations to be measured using fluorescence microscopy, flow cytometry and fluorescence spectroscopy. Examples of fluorescent calcium indicators are Bis-fura, BTC, calcium-green-1, calcium green-2, calcium green-5N, calcium orange, calcium crimson, Fluo-3, Fluo-4, Fluo-5, Fluo-5F, Fluo-4FF, Fluo-5N, Fura-2, Fura-5F, Fura-4F, Fura-6F, Fura-FF, Fura Red, Indo-1, Indo-5F, Mag-fluo-4, Mag-fura-5, mag-indo-1, magnesium Green, Oregon Green, Quin-2, Rhod-2, Rhod-FF, Rhod-5N, X-rhod-1, X-rhod-5F and X-rhod-FF. The fluorescence dye may be measured by Molecular Device's FLIPR with an argon laser (excitation at 488 nm) using the FLIPR Calcium assay kit (Molecular Devices) Calcium influx may also be monitored light emitting indicators such as assays using the luminescent protein aequorin.

Increased influx of calcium into a cell is toxic and will induce cellular apoptosis. Apoptosis is programmed cell death that is characterized by morphological changes within the cell. These morphological changes include shrinking and condensation of the nucleus, and fragmentation of the DNA. Apoptosis is detected by observing and quantitating these morphological changes with standard techniques.

Cells of the Invention

In some embodiments of the invention, a test agent is screened to determine whether it alters the function of NMDA receptors expressed by a cell in culture, or a solution containing an NMDA receptor subunit or a NMDA receptor complex. In one embodiment, the methods of identifying modulators of NMDA receptor function comprises contacting a test agent with a cell expressing a NMDA receptor subunit, preferably subunit NR2 and the central components of the NRG1 signaling pathway. The central components of the NRG1 signaling pathway are ErbB4, Fyn and Pyk2. Cells known to endogenously express the central components of the NRG1 signaling pathway and the NMDA receptor complex may be used in the methods of the invention.

Preferably, the methods will use neuronal cell lines such as HCN-1A (ATCC Accession No. CRL-10442), HCN-2 (ATCC Accession No. CRL-10742), A2B5 (ATCC Accession No. CRL-1573), SK-N-AS (ATCC Accession No. CRL-2142), SK-N-FI (ATCC Accession No. CRL-2142), and SK-N-DZ (ATCC Accession No. CRL-2149) and Daoy (ATCC Accession No. HTB-186). In particular, BE(2)-M17 neuroblastoma cells (ECACC No. 95011816) which have a neuron-like appearance after differentiation induced by retinoic acid and are known to express ErbB4 and the NMDA receptor subunits NR1 and NR2B. Another example of a preferable cell line to use in the present invention is SH-SY5Y (ATCC Accession No. CRL-2266).

Cell transfected to express NMDA receptor subunits and/or NRG1 signaling pathway components may be used in methods of the present invention. A number of transfection techniques are well known in the art such as calcium phosphate transfection, DEAE, dextran mediated transfection, lipofectamine transfection or electroporation (Davis et al., Basic Methods in Molecular Biology, 1986; Sambrook et al., Molecular Cloning, a laboratory Manual, Cold Spring Harbor Laboratories (New York, 1989); Davis et al., Basic Methods in Molecular Biology, Elsevier, 1986; and Chu et al., Gene, 0.1:3:197, 1981).

The methods of the present invention may be carried our with cells transfected or transformed to express a NMDA receptor complex and the NRG1 signaling components. For a cell to be used in the methods of the invention, the cell must endogenously or be transfected or transformed to express at a minimum a NMDA receptor subunit NR2 and Fyn and/or optionally ErbB4 and Pyk2. The invention provides for nucleic acid constructs containing a nucleic acid molecules that encode these polypeptides. The constructs comprise a vector (e.g., an expression vector) into which one or more sequences of the NRG1 signaling pathway have been inserted in a sense or antisense orientation. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked. In general, expression vectors of utility in recombinant DNA techniques are often in the form of plasmids. However, the invention is intended to include such other forms of expression vectors, such as viral vectors (e.g., replication defective retroviruses, adenoviruses and adeno-associated viruses) that serve equivalent functions.

Preferred recombinant expression vectors comprise a nucleic acid molecule that encodes a desired polypeptide in a form suitable for expression of the polypeptide in a host cell. Therefore, recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). The design of the expression vector can depend on such factors as the choice of the host cell to be transformed and the level of expression of polypeptide desired. The expression vectors can be introduced into host cells to thereby produce polypeptides, including fusion polypeptides, encoded by nucleic acid molecules as' described herein.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein. Aspects of the invention described by reference to a cell should be understood to embrace embodiments using multiple cells.

Vector DNA can be introduced into prokaryotic or eukaryotic cells via conventional transformation or transfection techniques. As used herein, the terms “transformation” and “transfection” are intended to refer to a variety of art-recognized techniques for introducing a foreign nucleic acid molecule (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. Molecular Cloning, a laboratory manual, Cold Spring Harbor Laboratories (New York, 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those that confer resistance to drugs, such as G418, hygromycin and methotrexate. Nucleic acid molecules encoding a selectable marker can be introduced into a host cell on the same vector as the nucleic acid molecule of the invention or can be introduced on a separate vector. Cells stably transfected with the introduced nucleic acid molecule can be identified by drug selection (e.g., cells that have incorporated the selectable marker gene will survive, while the other cells die).

A host cell of the invention, such as a prokaryotic or eukaryotic host cell in culture, can be used to produce (i.e., express) polypeptides described herein for both assays using the host cells of the invention and assays using isolated polypeptides. Standard methods comprising culturing the host cell of invention (into which a recombinant expression vector encoding a polypeptide of the invention has been introduced) in a suitable medium such that the polypeptide is produced, and optionally isolating the polypeptide from the medium or the host cell, are contemplated.

Polynucleotides and Polypeptides

The invention provides methods that use cells expressing a NMDA receptor complex. These cells may be transfected or transformed to express the NMDA receptor complex, including subunits NR1, NR2A, NR2B, NR2C and NR2D. To express the human NMDA receptor subunit NR1, cells may be transformed or transfected with the polynucleotide sequence of SEQ ID NO: 1 (Genbank Accession No. LI 3266) or any polynucleotide sequence that encodes the amino acid sequence of SEQ ID NO: 2 (Genbank Accession No. AAB59360). To express the human NMDA receptor subunit NR2A, cells may be transformed or transfected with the polynucleotide sequence of SEQ ID NO: 3 (Genbank Accession No. NM_{—}000833) or any polynucleotide sequence that encodes the amino acid sequence of SEQ ID NO: 4 (Genbank Accession No. NP_{—}000824). To express the human NMDA receptor subunit NR2B, cells may be transformed or transfected with the polynucleotide sequence of SEQ ID NO: 5 (Genbank Accession No. NM_{—}000834) or any polynucleotide sequence that encodes the amino acid sequence of SEQ ID NO: 6 (Genbank Accession No. NM_{—}000825). To express the human NMDA receptor subunit NR2C, cells may be transformed or transfected with the polynucleotide sequence of SEQ ID NO: 7 (Genbank Accession No. NM_{—}000835) or any polynucleotide sequence that encodes the amino acid sequence of SEQ ID NO: 8 (Genbank Accession No. NM_{—}000826). To express the human NMDA receptor subunit NR2D, cells may be transformed or transfected with the polynucleotide sequence of SEQ ID NO: 9 (Genbank Accession No. NM_{—}000836) or any polynucleotide sequence that encodes the amino acid sequence of SEQ ID NO: 10 (Genbank Accession No. NM_{—}000827). The cells may also be transfected or transformed to express variants or fragments of the NR1 and NR2 subunits, wherein these variants and fragments retain the ability to form a heterodimeric ionotrophic channel that binds glutamate and glycine. Practice of the invention with human allelic variants including variants that correlate with increased risk of schizophrenia are specifically contemplated.

The invention also contemplates using cells transfected or transformed with other mammalian NMDA receptor subunits other than a the human NMDA receptor subunits. These mammalian NMDA receptor subunit NR1 polypeptides include mouse NR1 polypeptide (Genbank Accession No. BAA00920) and rat NR1 polypeptide (Genbank Accession No. NP_{—}058706.1). These mammalian NMDA receptor subunit NR2A polypeptides include mouse NR2A polypeptide (Genbank Accession No. BAA01069) and rat NR2A polypeptide (Genbank Accession No. AAC03565). These mammalian NMDA receptor subunit NR2B polypeptides include mouse NR2B polypeptide (Genbank Accession No. NP_{—}032197) and rat NR2B polypeptide (Genbank Accession No. AAA41714.1). These mammalian NMDA receptor subunit NR2C polypeptides include mouse NR2C polypeptide (Genbank Accession No. NP_{—}034480.1) and rat NR2C polypeptide (Genbank Accession No. BAA02499.1). These mammalian NMDA receptor subunit NR2D polypeptides include mouse NR2D polypeptide (Genbank Accession No. NP_{—}032198) and rat NR2D polypeptide (Genbank Accession No. BAA02500.1).

To express the human ErbB4 polypeptide, cells may be transformed or transfected with the polynucleotide sequence of SEQ ID NO: 11 (Genbank Accession No. L07868) or any polynucleotide sequence that encodes the amino acid sequence of SEQ ID NO: 12. (Genbank Accession No. AAB59446). The extracellular domain contains the ligand binding site. The protein has a single transmembrane domain that anchors it to the plasma membrane. The intracellular domain (amino acids 676-1308 of SEQ ID NO: 12) contains the tyrosine kinase (amino acids 713-988 of SEQ ID NO: 12) and examples of tyrosine residues that are phosphorylated on ErbB4 include Y1056, Y1188 and Y1242. Some methods of the invention use cells expressing ErbB4 polypeptide. The cells may also be transfected or transformed to express variants or fragments of the ErbB4 polypeptide, wherein these variants and fragments retain the ability to bind NRG1.

To express human Fyn polypeptide, cells may be transformed or transfected with the polynucleotide sequence of SEQ ID NO: 13 (Genbank Accession No. NM_{—}002037) or any polynucleotide sequence that encodes the amino acid sequence of SEQ ID NO: 14 (Genbank Accession NO. NP_{—}002028). To express human Pyk2 polypeptide, cells may be transformed or transfected with the polynucleotide sequence of SEQ ID NO: 15 (Genbank Accession No. U33284) or any polynucleotide sequence that encodes the amino acid sequence of SEQ ID NO: 16 (Genbank Accession No. AAC50203.1).

The invention also contemplates using cells transfected or transformed with other mammalian NRG1 signaling pathway components other than human NRG1 signaling pathway components. The invention contemplates using cells transfected or transformed to express other mammalian ErbB4 polypeptide. These mammalian ErbB4 polypeptides include mouse ErbB4 polypeptides (Genbank Accession Nos. AAA93534.1, AAC288333.1 and XP136682.3) and rat ErbB4 polypeptides (Genbank Accession Nos. NP_{—}0677350.1, AAQ77349.1; AAQ77348.1 and AAD08899.1). The invention also contemplates using cells transfected or transformed to express other mammalian Fyn polypeptides. These mammalian Fyn polypeptides include mouse Fyn polypeptide (Genbank Accession No. AAH32149). These mammalian Pyk2 polypeptides include mouse Pyk2 (Genbank Accession No. Q9QVP9) and rat Pyk2 polypeptide (Genbank Accession No. NP_{—}059014).

Sequence Variants

The invention contemplates using variants or fragments of any of the above-described NMDA receptor complex proteins and any of the NRG1 signaling pathway components, and polynucleotides encoding them. The description of these variants and fragments herein refers to all of the polypeptides of the invention. The term “polypeptide” refers to a polymer of amino acids, and not to a specific length; thus, peptides, oligopeptides and proteins are included within the definition of a polypeptide. As used herein, a polypeptide is said to be “isolated” or “purified” when it is substantially free of cellular material when it is isolated from recombinant and non-recombinant cells, or free of chemical precursors or other chemicals when it is chemically synthesized. A polypeptide, however, can be joined to another polypeptide with which it is not normally associated in a cell (e.g., in a “fusion protein”) and still be “isolated” or “purified.”

Variants include a substantially homologous polypeptide encoded by the same genetic locus in an organism, i.e., an allelic variant, as well as other splicing variants. Preferred variants are biologically active. Variants also include polypeptides substantially homologous or identical to these polypeptides but derived from another organism, i.e., an ortholog. Variants also include polypeptides that are substantially homologous or identical to these polypeptides that are produced by chemical synthesis. Variants also include polypeptides that are substantially homologous or identical to these polypeptides that are produced by recombinant methods.

Preferred polypeptides have an amino acid sequence at least 80% identical to a human or other mammalian sequence described herein or in a public database or later discovered, or a fragment of such sequences, so long as the polypeptide retains either wild type function or, in some embodiments, reduced function characteristic of a schizophrenia phenotype in humans. Fragments and variants that are more similar, e.g., at least 85%, 90%, 91%, 92%, 93%, 94%, 95%, 96%, 97%, 98%, 99%, 99.5%, or 100%, are highly preferred. Fragments or variants that are at least 35%, 40%, 45%, 50%, 55%, 60%, 65%, 70%, and 75% are also contemplated. A genus of similar polypeptides can alternatively be defined by the ability of encoding polynucleotides to hybridize to the complement of a nucleotide sequence that corresponds to the cDNA sequence encoding the polypeptide.

Preferred polypeptides may be described as having an amino acid sequence encoded by a nucleic acid sequence at least 80% identical to a full length or fragment of a naturally occurring sequence or cDNA sequence. Nucleic acids that are more similar, e.g., at least 85%, 90%, 91%, 92%, 93%, 94%, 95%, 96%, 97%, 98%, 99%, 99.5%, or 100% are highly preferred. Nucleic acids that are 35%, 40%, 45%, 50%, 55%, 60%, 65%, 70%, and 75% are also contemplated.

Hybridization stringency is principally determined by temperature, ionic strength, and the concentration of denaturing agents such as formamide. Examples of “highly stringent conditions” for hybridization and washing are 0.015M sodium chloride, 0.0015M sodium citrate at 65-68° C. or 0.015M sodium chloride, 0.0015M sodium citrate, and 50% formamide at 42° C. See Sambrook, Fritsch & Maniatis, Molecular Cloning: A Laboratory Manual, 2^{nd} Ed., Cold Spring Harbor Laboratory, (Cold Spring Harbor, N.Y. 1989).

When selecting variant sequences to practice the invention, it will be understood that conservative substitutions sequence changes constitute a preferred class of substitutions. Such substitutions are those that substitute a given amino acid in a polypeptide by another amino acid of like characteristics. Conservative substitutions are likely to be phenotypically silent. Typically seen as conservative substitutions are the replacements, one for another, among the aliphatic amino acids Ala, Val, Leu and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe and Tyr. Guidance concerning which amino acid changes are likely to be phenotypically silent are found in Bowie et al., Science 247:1306-1310 (1990).

A variant polypeptide can differ in amino acid sequence by one or more substitutions, deletions, insertions, inversions, fusions, and truncations or a combination of any of these. Further, variant polypeptides can be fully functional or can lack function in one or more activities. Fully functional variants often contain mostly conservative variation or variation in residues or regions less important for biological activity. Functional variants can also contain substitution of similar amino acids that result in no change or an insignificant change in function. Alternatively, such substitutions may positively or negatively affect function to some degree. Non-functional variants typically contain one or more non-conservative amino acid substitutions, deletions, insertions, inversions, or truncation or a substitution, insertion, inversion, or deletion in a critical residue or critical region.

Amino acids that are essential for function can be identified by methods known in the art, such as site-directed mutagenesis or alanine-scanning mutagenesis (Cunningham et al., Science, 244:1081-1085-1989). The latter procedure introduces single alanine mutations at every residue in the molecule. The resulting mutant molecules are then tested for biological activity in vitro, or in vitro proliferative activity. Sites that are critical for polypeptide activity can also be determined by structural analysis such as crystallization, nuclear magnetic resonance or photoaffinity labeling (Smith et al., J. Mol. Biol., 224:899-904, 1992; de Vos et al. Science, 255:306-312, 1992).

The invention also includes polypeptide fragments of the polypeptides of the invention. Fragments can be derived from a polypeptide encoded by a nucleic acid molecule of the present invention or a portion thereof or the polypeptide having the amino acid sequence of the present invention. Biologically active fragments (peptides which are, for example, 6, 9, 12, 15, 16, 20, 30, 35, 36, 37, 38, 39, 40, 50, 100 or more amino acids in length) can comprise a domain, segment, or motif that has been identified by analysis of the polypeptide sequence using well-known methods, e.g., signal peptides, extracellular domains, one or more transmembrane segments or loops, ligand binding regions, kinase domains, DNA or protein binding domains, acylation sites, glycosylation sites, or phosphorylation sites. Enzymatically active fragments can comprise a domain, segment, or motif that has been identified by analysis of an enzyme using well-known methods, as described above. Such biologically active fragments or enzymatically active fragments can be identified using standard means for assaying activity of a polypeptide or enzyme.

It is also contemplated that embodiments of the invention can be practiced with chimeric or fusion polypeptides. These comprise a polypeptide of the invention operatively linked to a heterologous protein or polypeptide having an amino acid sequence not substantially homologous to the polypeptide. “Operatively linked” indicates that the polypeptide and the heterologous protein are fused in-frame. The heterologous protein can be fused to the N-terminus or C-terminus of the polypeptide. In one embodiment the fusion polypeptide does not affect function of the polypeptide per se. For example, the fusion polypeptide can be a GST-fusion polypeptide in which the polypeptide sequences are fused to the C-terminus of the GST sequences. Other types of fusion polypeptides include, but are not limited to, enzymatic fusion polypeptides, for example β-galactosidase fusions, yeast two-hybrid GAL fusions, poly-His fusions and Ig fusions. Such fusion polypeptides, particularly poly-His fusions, can facilitate the purification of recombinant polypeptide. In certain host cells (e.g., mammalian host cells), expression and/or secretion of a polypeptide can be increased by using a heterologous signal sequence. Therefore, in another embodiment, the fusion polypeptide contains a heterologous signal sequence at its N-terminus.

A chimeric or fusion polypeptide can be produced by standard recombinant DNA techniques. For example, DNA fragments coding for the different polypeptide sequences are ligated together in-frame in accordance with conventional techniques. In another embodiment, the fusion gene can be synthesized by conventional techniques including automated DNA synthesizers. Alternatively, PCR amplification of nucleic acid fragments can be carried out using anchor primers which give rise to complementary overhangs between two consecutive nucleic acid fragments which can subsequently be annealed and re-amplified to generate a chimeric nucleic acid sequence (see Ausubel et al., Current Protocols in Molecular Biology, 1992). Moreover, many expression vectors are commercially available that already encode a fusion moiety (e.g., a GST protein). A nucleic acid molecule encoding a polypeptide of the invention can be cloned into such an expression vector such that the fusion moiety is linked in-frame to the polypeptide.

The isolated polypeptide can be purified from cells that naturally express it, purified from cells that have been altered to express it (recombinant), or synthesized using known protein synthesis methods. In one embodiment, the polypeptide is produced by recombinant DNA techniques. For example, a nucleic acid molecule encoding the polypeptide is cloned into an expression vector, the expression vector introduced into a host cell and the polypeptide expressed in the host cell. The polypeptide can then be isolated from the cells by an appropriate purification scheme using standard protein purification techniques.

Test Agents of the Invention

Test agents of the present invention can be any organic or inorganic molecule, complex or substance. Exemplary test agents are obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to polypeptide libraries, while the other four approaches are applicable to polypeptide, non-peptide oligomer or small molecule libraries of compounds (Anticancer Drug Des., 12:145, 1997). Numerous chemical libraries already exist, e.g., as proprietary libraries of pharmaceutical companies, and compounds in such libraries are suitable test agents. Test agents of the invention include fusion proteins, polypeptides, peptidomimetics, prodrugs, receptors, binding agents, ribozymes, small molecules, peptides, antibodies, or other drugs which can be screened for the ability to modulate NMDA receptor function or NMDA receptor complex phosphorylation.

In embodiments of the invention, test agents will be administered to a mammal to screen for modulators of NMDA receptor function. For administration to a mammal, the test agent may be formulated with a pharmaceutically or physiologically acceptable carrier or expedient. The carrier and test agent may be sterile and the formulation should suit the mode of administration. The test agent may be administered to the mammal using methods of introduction conventional in the art including, but not limited to, intradermal, intramuscular, intraperitoneal, intraocular, intravenous, subcutaneous, topical, oral and intranasal administration.

Models of Schizophrenia

The present invention provides methods of identifying modulators of NMDA receptor function wherein reduced NMDA function is indicative of schizophrenic condition. In some embodiments, the methods are practiced with an animal model of schizophrenia. Two genetic mouse models that are exemplified in the present application are NRG1 heterozygotic mice and ErbB4 heterozygotic mice. Genetic models are discussed in greater detail below. The same animal model optionally can be used to study both biochemical and behavioral indicia of schizophrenia and the effects of test compounds on each.

Behavioral Assays

Some aspects of human schizophrenia may be measured in animal models and measuring these behavioral traits has proved to be a valid approach for studying schizophrenia. The term “behavioral symptom of schizophrenia” refers to a behavioral trait in an animal that mimics a clinical aspect of schizophrenia in humans. These behavioral symptoms include increased psychomotor activity or hyperactivity, stereotyped behavior, decreased social activity or social withdrawal, impaired cognitive, primarily executive, functions relating to prefrontal cortical function and impaired sensorimotor gating (i.e., P50 auditory gating).

Impaired social interactions and social withdrawal have been shown as key features of schizophrenia. Altered social behavior has been identified as early marker of behavioral abnormalities before the appearance of the full-blown psychosis. Social withdrawal was shown in rhesus monkeys injected with amphetamine as one of the drug-induced animal models of schizophrenia. More recently, mice with genetically lower expression of NMDR receptor subunit NR1, described in detail below, showed altered social behavior that could be normalized by the atypical antipsychotic drug clozapine.

To measure social dysfunction, home cage observation may be carried out. Mice exhibit a specific behavioral repertoire when they meet with the same-sex in their home cage. This behavior includes approach, avoidance, aggressive and non-aggressive interactions. Tests are available to analyze this aspect of the disorder by carefully analyzing the interaction between two or more mice (Lijam et al. Cell, 905: 895-905, 1997). Group-housed mice are observed for two, two-hour periods in one day, during the light and the dark periods. During this time behavior is recorded and later is analyzed by an experienced blind observer. The social aspects of the behavior focused on include huddling, social grooming, tail pulling, aggression, and associated behaviors such as genital sniffing. In addition, nesting behavior is observed and ability to create nests is assessed.

Social dysfunction may also be analyzed by social investigation that allows the careful analysis of the interaction between two mice in a neutral arena (open field for 30-40 minutes). Whereas the behavior of the “observed” mouse is controlled by the use of restrain (wire mesh cage in the center of the arena) the behavior of the experimental and control observer mice are carefully recorded.

Impaired sensorimotor gating is another behavioral symptom of schizophrenia observed in animal models. The acoustic startle is an unconditioned reflex response to external auditory stimulation. Prepulse inhibition (PPI), which consists of a reduced startle response to auditory stimulation following presentation of a weak auditory stimulus, has been used as a tool for the assessment of deficiencies in sensory-motor gating, such as those seen in schizophrenia (Paylor & Crawley, Pyschopharmacology, 132(2): 169-180, 1997). PPI is impaired in schizophrenic patients, and is considered a test with good predictive and construct validity for schizophrenia. Impaired PPI is also associated with cognitive deficits such as distractibility and thought disorder in schizophrenic patients.

Sensorimotor gating may be analyzed by measuring in PPI and habituation. There is some evidence that schizophrenic patents have a deficit in habituation, where habituation refers to the decrease in the unconditioned response to the repeated presentation of a stimulus (Braff et al., Arch gen. Psychiatry 49(3): 260-215, 1992). Habituation can be studied in the mouse by repeatedly presenting an auditory signal in a startle chamber and measuring the decline in a startle response, or by studying the habituation of an exploratory response to a novel environment (Brodkin, Behav. Pharmacol., 10(5): 445-451, 1999). Habituation of the startle response is measured in mice by exposing them to a series of loud sounds (50 trials of a 120 dB sound). PPI and their startle levels are measured and the rate of habituation within a session is assessed (Geyer & Braff, Schizophrenia Bull. 13(4): 643-668, 1987).

Sensorimotor gating may be analyzed by measuring prepulse inhibition of startle with clozapine. Mice harboring a genetic defect and wild type were injected with 2-3 doses of clozapine and a vehicle. Adding a pharmacological agent to the prepulse inhibition of startle allows assessment of the sensitivity of the mutant mice to a drug that affects various neurotransmitters systems, assumed to be involved in the pathology of schizophrenia. A differential drug response in the mutant mice may indicate altered functional activity of the major neurotransmitter systems thought to be involved in schizophrenia.

The hyperlocomotion induced by PCP and amphetamines in mice has been proposed to mimic the psychosis found in schizophrenia. To assess drug-induced hyperlocomotion, studies measuring PCP or amphetamine-induced locomotion in a dimly light open field are carried out. The open field is a standard test in which spontaneous activity can be measure. Running this test in the dark will help reduce the influence of factors such as visual acuity and anxiety-like responses. Animals will be placed in a square open field box and their behavior will be recorded for 30 minutes. Overall activity will be recorded. After this first session in the open field, animals will be picked up and injected with a dose of amphetamine or vehicle 20 minutes after the injection, activity will be recorded.

It has been proposed that schizophrenic patients have attentional deficits that impair the conscious processing of information. Attentional problems can be construed as deficit in allocation of one or more pools of attentional resources to attend a certain task. This can lead to profound alterations in the information processing that may present itself clinically as psychotic thoughts. The most common test to measure sustained attention in humans is the continuous performance task (CPT) that has been used to measure attentional deficits in schizophrenics and their relatives (Walker & Shaye, Arch Gen Psychiatry 39(10): 1153-1156, 1982). In schizophrenics, some aspects of their performance on the CPT test can improved by classical neuroleptics (Nestor et al. Neuropsychopharmacy 4(2): 145-149, 1991; Liu et al. Eur. J. Pharmacol. 398(3): 333-339, 2000)

The Continuous Performance Test in animal provides a test that is analogous to the human Continuous Performance Test (CPT) that is used to measure impulsivity (as behavioral disinhibition) and sustained attention in humans, monkeys and rats (Rutschmann et al., Arch Gen Psychiatry 141(1); 57-65, 1977; Muir et al., Cereb. Cortex 6(3): 470-481, 1996). In the human version responding on a keyboard is correct only if it occurs during the presentation of the letter “K”. Responding when no signal is present is a “commission error” and no responding during the signal is an “omission error”. Correct responses are “hits”. Whereas commission errors are a measure of motor impulsivity or hyperactivity (more impulsivity or hyperactivity, higher commission error rate), omission errors are a measure of inattention (less attention, more omission errors). There are three versions of this test which differ in the number of possible responses (1, 2 or 3). The three-response version is called the 3-hole attentional task. This task is very similar to the five-hole attentional task, commonly use in rats (Baunez & Ribbins, Psychopharmacology, 141(1): 57-65, 1999) with the only difference being the number of holes. In this test, subjects must maintain attention throughout the session (sustained attention) and respond only when a cue (light) indicates so. In the 3-hole attentional task there are four possible outcomes; responses to the right hole, response to the wrong hole #1, response to the wrong hole #2, no response.

Cognitive deficits are also contemplated as a behavioral symptom of schizophrenia in animal models. Time perception is a useful measure of cognitive function, sensitive to dopaminergic and cholinergic manipulations. As in all behavioral tasks, several processes underlie good steady state performance in a temporal task: attention, motivation, short and long term memory, motor coordination and instrumental learning. Three main types of temporal tasks exist: (i) in “Scaling”, subjects must, for example, categorize a stimulus into a given set of categories or verbally estimate the duration, (ii) in “Discrimination” a comparison is made between two durations, and (iii) in “Reproduction”, a response is made that bears some relation with the stimulus). Timing in both animals and humans is very sensitive to both dopaminergic and cholinergic manipulations.

Spontaneous alternation in the Y maze to measure working memory is also analyzed to assess cognitive deficits. Mice have an innate tendency to explore their environment. A successful exploration depends on the ability to remember places recently visited and avoiding repeat visits. The advantage of this test is that it is ethologically based and does not involve food or other types of rewards. In this test, mice are placed in a Y maze for a period of 8 minutes and their activity is recorded. This includes number of visits in each arm and the order of arm visits. Global activity was assessed by measuring the number of visits to the different arms. Simple alternation is defined as a visit to one arm followed by a visit to an adjacent arm. In the proposed design mice could repeat entries into a single arm, resulting in a chance performance level of 22% alternations. Percentage alternation will be calculated by dividing the number of triads containing entries into all three arms by alternation opportunities X 100.

Animal Models

One variation of the methods of the invention contemplates using genetic animal models of schizophrenia. Any animal harboring a genetic defect in the NRG1 signaling pathway is useful for carrying out the methods of the invention. Preferably, the animal harboring the genetic defect will have a NMDA receptor complex hypophosphorylation phenotype. The term “NMDA receptor complex hypophosphorylation phenotype” refers to an animal which has a reduced level of phosphorylation of one or more proteins of the NMDA receptor complex, compared to a measurement of phosphorylation of the same protein in a wild type animal of the same species. Examples of animals exhibiting a NMDA receptor complex hypophosphorylated phenotype include the NRG1 heterozygotic mice generated by Harvey et al. and described in Steffanson et al., Am. J. Hum. Genet. 72: 83-7, 2003, and those NRG1 heterozygotic mice described in Erickson et al., Development, 24(24): 4999-5011, 1997.

The methods of the invention may also be carried out with a mammal having a NRG1 signaling protein hypophosphorylated phenotype. The term “NRG1 signaling pathway protein hypophosphorylated phenotype” refers to an animal which has a reduced level of phosphorylation of one or more protein components of the NRG1 signaling pathway, compared to measurement of phosphorylation of the same protein in a wild type animal of the same species. Examples of animals exhibiting a NRG1 signaling protein hypophosphorylated phenotype include ErbB4 heterozygous mice described in Gassmann et al. (Nature 378(6555): 390-4, 1995).

Animals having a NMDA receptor complex hypophosphorylated phenotype or a NRG1 signaling pathway protein hypophosphorylated phenotype may be identified by measuring the level of phosphorylation in the brain homogenates of the animal using conventional methods in the art and those described herein. For example, phosphorylation may be measured by immunoprecpitation of proteins of the NMDA receptor complex or the NRG1 signaling pathway and detecting phosphorylation by Western blot analysis using phospho-specific antibodies, such as anti-phosphotyrosine antibodies and anti-phosphoserine/phosphothreonine antibodies. In addition, mass spectrometry may be used to measure the phosphorylated proteins.

NRG1 hypomorphic mutant mice (NRG1^{+/-}) are heterozygotic mice that have a mutant NRG1 allele in which the NRG1 exon encoding the transmembrane domain is disrupted in the heterozygote. These mice develop normally and breed well. In behavioral tests, the NRG1^{+/-} mice exhibited an abnormal phenotype. In an open field test under dim red light, these mice were significantly more active than the wild type mice but did not differ in measures of anxiety such as time in the center of the area. Treatment of the NRG1^{+/-mice with a clozapine (}1 mg/kg), an atypical antipsychotic drug commonly used to treat schizophrenia, for 25 minutes prior to testing reversed the behavioral abnormality. Clozapine (1 mg/kg) did not affect the activity of the litter-mate control mice; thus, it was not sedating at the dose used in the test. In addition, it is known that the NRG1^{+/-} had 16% fewer functional NMDA receptors overall as compared to litter-mate controls as determined by MK-801 (NMDA antagonist) binding studies on forebrain homogenates (Steffanson et al., Am. J. Hum. Genet. 72: 83-7, 2003). Therefore, these mice are relevant as an in vivo model for schizophrenia.

Additional NRG1 heterozygotic mice are described in Erickson et al., Development, 24(24): 4999-5011, 1997. These mice were generated by replacing the exon encoding the N-terminal half of the EGF domain with a neomycin cassette. This null mutation results in disruption of all splice forms of NRG1. Homozygotic null mice were embryonically lethal and had developmental defects in cardiac abnormalities and abnormalities in the cranial nerves and ganglia. Theses NRG1 heterozygotic mice also exhibited significant elevated activity in open field tests and T-maze tests Gerlai et al. (Behav. Brain Res. 109: 219-227, 2000). It is contemplated that the NRG1 hypomorphic mice may be used in the methods of identifying anti-schizophrenic agents and modulators of NMDA receptor function of the invention.

The experiments described in Example 3 are also carried out on ErbB4^{+/-} heterozygotic mice generated as described in Gassmann et al. (Nature 378(6555): 390-4, 1995). These mice develop normally, bred well, but showed abnormal behavior. Similar to the NRG1^{+/-} mice, the ErbB4^{+/-} mice were significantly more active than wild type mice in the open field-test performed under dim red light. As the NRG1 signaling pathway is initiated by binding to ErbB4, the ErbB4^{+/-} mice are contemplated to be relevant as an in vivo model for schizophrenia. In addition, there is ErbB4 heterozygotic mouse generated by Long et al. (Development 130: 5257-68, 2003) which has a loxP site inserted in intron I and another in intron 2 of the ErbB4 gene, thus flanking exon 2. A conditional homozygous ErbB4 knock out mouse was generated by Golub et al. Behav Brain Res. 153(1):159-70, 2004. These mice lacking both alleles of the erbB4 gene in neurons/glia and these mice exhibit a lower level of spontaneous motor activity and reduced grip strength compared to wild type mice. Heterozygous nulls were delayed in motor development and male heterozygous nulls demonstrated altered cue use in a Morris maze learning and memory task. The invention contemplate using the ErbB4^{+/-} mice to carry out the methods of identifying anti-schizophrenic agents and modulators of NMDA receptor function of the present invention.

The experiments described in Example 1 demonstrate that NRG1 signaling involves the ErbB4 binding protein Fyn. Fyn^{-/-} (null) mutant mice are known to have defects in NMDA receptor-dependent long term potentiation and spatial memory (Grant et al., Science 258: 1903-1910, 1992). In addition, the Fyn^{-/-} mice have reduced tyrosine phosphorylation of the NR2A and NR2B subunits. (Tezuka et al., Proc. Natl. Acad. U.S.A., 96: 735-440, 1999), and are known to exhibit various neural defects. (Nakazawa et al., J. Biol. Chem. 276: 693-699, 2001) As Fyn is shown to play a role in NRG1 signaling, the Fyn^{-/-} mice may also be relevant as an in vivo model for schizophrenia. It is contemplated that the Fyn^{-/-} mice may be used in the methods of identifying anti-schizophrenic drugs or methods of modulating NMDA receptor function of the present invention.

Additional genetic animal models of the glutamatergic hypothesis of schizophrenia include NMDA receptor subunit NR1 hypomorphic (or knock-down) mice (Mohn et al., Cell 98: 427-436, 1999) and NMDA receptor subunit NR2A subunit null mice (Miyamoto et al., J. Neurosci. 21: 750-757, 2001). The NR1 hypomorphic mice exhibit moderate hyperactivity and a deficit in social interactions that are responsive to anti-psychotic drugs such as clozapine. The NR2A null mice exhibited moderate hyperactivity and deficits in learning and eye blink conditioning. Thus, these animal models may also be used in the methods of identifying anti-schizophrenic drugs and methods of identifying modulators of NMDA receptor function of the present invention.

Other genetic models of schizophrenia include the “Reeler mice.” These mice have a spontaneous mutation in the reelin protein and exhibit neurodevelopmental abnormalities such as misplacement of neurons and associated ataxia. The reelin protein is a large extracellular protein that is secreted by pioneer neurons that coordinate cell positioning during neurodevelopment. Studies have indicated that there is a reduction in reelin immunoreactivity in the hippocampus of schizophrenic patients. The heterozygotic Reelin mice exhibit postpubertal appearance of sensory-motor gating (PPI) deficits, deficits in hippocampus-specific cognitive functions, selective decrease of cortical dentritic spine density and a selective decrease of nicotiniamideiadenine dinucleotide phosphate positive GABA-ergic neurons in superficial white matter but an increase in deeper white matter. These findings closely resemble neuromorphical and behavioral abnormalities seen in schizophrenic patients. Two additional autosomal recessive mutant mice, scrambler and yotari exhibit phenotypes similar to the Reeler mice. These mice have a mutation in the mouse gene, mdab1, which is related to the Drosophila gene disabled (dab). These mice are available from Jackson Labs (Bar Harbor, Me.).

Deletions or rearrangements of human chromosome 2q11 lead to a variety of clinical symptoms such as DiGeorge syndrome and velo-cardiofacial syndrome. In addition, patients with deletions within 2q11 have an increased incidence of schizophrenia. Heterozygous mutant mice (known as DiGeorge mice) that have a deletion within the conserved DGS critical region on mouse chromosome 16 and exhibit an increase in prepulse inhibition (PPI) of startle response and a manifestation of sensorimotor gating. The DiGeorge mice are an animal model that may be used in the methods of the invention.

Another animal model for schizophrenia are proline-dehydrogenase mutant mice, which are available from Jackson Labs (Bar Harbor, Me.). These mice have a defect in sensorimotor gating (PPI) accompanied by regional neurochemical alterations in the brain dopaminergic systems. Another animal model of schizophrenia is mutant mice with a deletion in the catechol-O-methyltransferase (COMT) gene which is a candidate for the psychopathology of schizophrenia. Homozygotic female COMT knock out mice display a heightened anxiety-like behavior in the dark/light exploratory model of anxiety. Heterozygotic male COMT knock out mice exhibit increased aggressive behavior.

Serotonin (5HT)-2A receptors play a role in the pathophysiology of schizophrenia and the mechanism of action of atypical antipsychotics. 5HT-2A receptor knock out (5-HT-2A^{-/-}) mice are models of schizophrenia. Female 5HT-2A receptor knock mice have a reduced PPI. Mice that are deficient for Dv11, one of the mouse homologs for the Drosphila segment polarity gene Disheveleed, exhibit reduced social interaction and abnormal sensorimotor gating.

As an alternative to genetic models, animal models for schizophrenia for use in the invention can be created chemically. For example, other animal models for the glutamatergic hypothesis of schizophrenia include animals treated with moderate doses of NMDA receptor antagonists including but not limited to phencyclidine (PCP), ketamine, MK-801, dextrorphan, ethanol and memantine. Animals administered NMDA receptor antagonists display locomotor hyperactivity, deficits in social interactions, deficits in sensorimotor gating and cognitive deficits. The invention contemplates methods comprising administering an agent that reduces phosphorylation of the NMDA receptor subunit NR2, such as NMDA receptor antagonists, in the presence and absence of a test agent. The term “agent that reduces NMDA receptor complex phosphorylation” refers to any agent that reduces tyrosine or serine/threonine phosphorylation of the NMDA receptor complex.

Construction of Animal Models of Schizophrenia

In addition to the existing genetic models of schizophrenia taught herein, the methods of the invention may be carried out by generating transgenic mice that have reduced or null expression of any of the NRG1 signaling components, including ErbB4, Fyn or Pyk2. The invention provides for methods of making genetic animal models of schizophrenia. Such animals are themselves an aspect of the invention.

As noted above, a particular embodiment of the present invention provides transgenic animals heterozygous for a mutation in the NRG1 signaling pathway. Preferred animals exhibit a NMDA receptor complex hypophosphorylated phenotype. Transgenic animals expressing mutant NRG1 signaling component transgenes, recombinant cell lines derived from such animals, and transgenic embryos may be useful in methods for screening for and identifying agents that will be useful in treating or otherwise intervening in the events that lead to schizophrenia.

The transgenic animals of the present invention are constructed using an expression cassette employed to insert mutations in a NRG1 signaling pathway component gene of the animals, or to replace NRG1 signaling pathway component genes with orthologs, such as human wild-type and mutant alleles that correlate with NRG1 signaling pathway components.

Four basic approaches are contemplated for blocking expression of the NRG1 signaling pathway component gene: First, constructs may be designed to homologously recombine into particular endogenous gene loci, rendering the endogenous gene nonfunctional. Second, constructs are designed to integrate randomly throughout the genome. Third, constructs are designed to introduce nucleic acids complementary to a target endogenous gene. Expression of RNAs corresponding to these complementary nucleic acids will interfere with the transcription and/or translation of the target sequences. And fourth, constructs are designed to introduce nucleic acids encoding ribozymes, RNA-cleaving enzymes, that will specifically cleave a target mRNA corresponding to the endogenous gene. While homologous recombination is the preferred method, it is contemplated that the other strategies also may be useful for blocking NRG1 signaling pathway component gene expression.

Homologous Recombination

A desirable approach for blocking of endogenous NRG1 signaling pathway component protein production involves the use of homologous recombination. Homologous recombination relies, much like antisense described below, on the tendency of nucleic acids to base pair with complementary sequences. In this instance, the base pairing serves to facilitate the interaction of two separate nucleic acid molecules so that strand breakage and repair can take place. In other words, the “homologous” aspect of the method relies on sequence homology to bring two complementary sequences into close proximity, while the “recombination” aspect provides for one complementary sequence to replace the other by virtue of the breaking of certain bonds and the formation of others.

Put into practice, homologous recombination is used as follows. First, a target gene is selected within the host cell. Sequences homologous to the target gene are then included in a genetic construct, along with some mutation that will render the target gene inactive (stop codon, interruption, etc.). The homologous sequences on either side of the inactivating mutation are said to “flank” the mutation. Flanking, in this context, simply means that target homologous sequences are located both upstream (5′) and downstream (3′) of the mutation. These sequences should correspond to some sequences upstream and downstream of the target gene. The construct is then introduced into the cell, thus permitting recombination between the cellular sequences and the construct.

As a practical matter, the genetic construct will normally be designed to act as far more than a vehicle to interrupt the gene. For example, it is important to be able to select for recombinants and, therefore, it is common to include within the construct a selectable marker gene. This gene permits selection of cells that have integrated the construct into their genomic DNA by conferring resistance to various biostatic and biocidal drugs. In addition, a heterologous gene that is to be expressed in the cell also may advantageously be included within the construct.

Thus, using this kind of construct, it is possible, in a single recombinatorial event, to (i) “knock out” an endogenous gene, (ii) provide a selectable marker for identifying such an event and (iii) introduce a heterologous gene for expression. In the present invention, a preferred embodiment involves knocking out the endogenous mouse NRG1 signaling pathway component gene and replacing it with normal or mutant forms heterologous NRG1 signaling pathway component gene. Most preferably, normal or mutant forms of human NRG1 signaling pathway component are introduced into homozygous mice null for mouse NRG1 signaling pathway component. Of course, it is contemplated that the transgenic mice may be generated using heterologous NRG1 signaling pathway component genes from other organisms including but not limited to mammals such as sheep, cows, pigs, horses, rats, dogs cats, as well as other primates such as monkeys, apes, and baboons.

Another refinement of the homologous recombination approach involves the use of a “negative” selectable marker. This marker, unlike the selectable marker, causes death of cells which express the marker. Thus, it is used to identify undesirable recombination events. When seeking to select homologous recombinants using a selectable marker, it is difficult in the initial screening step to identify proper homologous recombinants from recombinants generated from random, non-sequence specific events. These recombinants also may contain the selectable marker gene and may express the heterologous protein of interest, but will, in all likelihood, not have the desired “knock out” phenotype. By attaching a negative selectable marker to the construct, but outside of the flanking regions, one can select against many random recombination events that will incorporate the negative selectable marker. Homologous recombination will likely not introduce the negative selectable marker, as it is outside of the flanking sequences.

In a first embodiment, a target NRG1 signaling pathway component gene within a host cell is selected as the target gene into which a selected gene is to be transferred. Sequences homologous to the target gene are included in the expression vector, and the selected gene is inserted into the vector such that target gene homologous sequences are interrupted by the selected gene or, put another way, the target gene homologous sequences “flank” the selected gene. In preferred embodiments, a drug selectable marker gene also is inserted into the target gene homologous sequences. Given this possibility, it should be apparent that the term “flank” is used broadly herein, namely, as describing target homologous sequences that are both upstream (5′) and downstream (3′) of the selected gene and/or the drug selectable marker gene. In effect, the flanking sequences need not directly abut the genes they “flank.” Application of a drug to such cells will permit isolation of recombinants.

On the other hand, site-specific recombination, relying on the homology between the vector and the target NRG1 signaling pathway component gene, will result in incorporation of drug selectable marker or other gene to replace the wild-type NRG1 signaling pathway component gene. Further screens for these phenotypes, either functional or immunologic, may be applied.

The Cre/Lox site specific recombination system from bacteriophage P1 (Sauer, Methods Enzymol., 225:890-900, 1993; Sauer, Methods, 14(4) 381-392, 1998, available through Gibco/BRL, Inc., Gaithersburg, Md.) may be used to remove specific genes out of a genome. Briefly, the system involves the use of a bacterial nucleotide sequence known as a LoxP site, which is recognized by the bacterial Cre protein. The Cre protein catalyzes a site specific recombination event. This event is bidirectional, i.e., Cre will catalyze the insertion of sequences at a LoxP site or excise sequences that lie between two LoxP sites.

Thus, if a construct containing a NRG1 signaling pathway component gene also has LoxP sites flanking the NRG1 signaling pathway component gene, introduction of the Cre protein, or a polynucleotide encoding the Cre protein, into the cell will catalyze the removal of the NRG1 signaling pathway component gene. In a further engineering step, it will be possible to employ a selectable marker to be inserted into the LoxP site vacated by the NRG1 signaling pathway component gene. This technique will thus yield a cell which is null for the NRG1 signaling pathway component that can be recognized by the expression of the selectable marker phenotype. This technology is explained in detail in U.S. Pat. No. 4,959,317, which is hereby incorporated by reference in its entirety. The various markers that may be employed are well known to those of skill in the art and certain examples are described in further detail elsewhere in the application.

Yet another system that may be used for homologous recombination is the flp/frt recombinase system from yeast. In a preferred embodiment, mouse genomic NRG1 signaling pathway component DNA is used to construct a knock-out mouse by homologous recombination. As such, flp recombinase can be expressed in mouse embryonic stem cells containing the targeting construct and will delete the NRG1 signaling pathway component gene or a portion thereof before introducing the stem cells into the recipient blastocyst.

Antisense Technology

In other preferred embodiments, the inventors use antisense oligonucleotides directed to the rat NRG1 signaling pathway component gene. Injection of these antisense oligonucleotides into the brains of rats may be an effective method of blocking the expression of the wild-type protein in those animals. Alternatively, the antisense sequences may be delivered through other techniques of gene transfer commonly employed in the art, such as, for example, viral transfer, receptor-mediated uptake, liposomal transfer and the like.

Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired With uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation whereas targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo.

Antisense constructs may be designed to bind to the promoter and other control regions, exons, introns or even exon-intron boundaries of a gene. It is contemplated that the most effective antisense constructs may include regions complementary to intron/exon splice junctions. Thus, antisense constructs with complementarity to regions within 50-200 bases of an intron-exon splice junction may be used. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

Random Integration

Although less specific than homologous recombination, there may be situations where random integration will be used as a method of knocking out a particular endogenous gene. Unlike homologous recombination, the recombinatorial event here is completely random, i.e., not reliant upon base-pairing of complementary nucleic acid sequences. Random integration is like homologous recombination, however, in that a gene construct, often containing a heterologous gene encoding a selectable marker, integrates into the target cell genomic DNA via strand breakage and reformation

Due to the lack of sequence specificity, the chances of any given recombinant integrating into the target gene are greatly reduced. As a result, it may be necessary to “brute force” the selection process. In other words, it may be necessary to screen hundreds of thousands of drug-resistant recombinants before a desired mutant is found. Screening can be facilitated, for example, by examining recombinants for expression of the selectable marker or even using immunologic or functional tests where the gene inserted to replace the NRG1 signaling pathway component gene is not a conventional selectable marker but is some other heterologous gene that may be advantageously incorporated into the genome.

Ribozymes

Ribozymes may be used to block endogenous NRG1 signaling pathway component protein production. Ribozymes are RNA-protein complexes that cleave nucleic acids in a site-specific fashion. Ribozymes have specific catalytic domains that possess endonuclease activity (Kim & Cech, Proc. Nat'l Acad. Sci. 84(24), 8788-92, 1987; Gerlach et al., Nature, 328:802-805, 1987; Forster and Symons, Cell 49(2):211-20, 1987; Forster and Symons, Cell 50(1) 9-16, 1987). For example, a large number of ribozymes accelerate phosphoester transfer reactions with a high degree of specificity, often cleaving only one of several phosphoesters in an oligonucleotide substrate (Cech et al., Cell, 27:487-96, 1981; Michel and Westhof, J. Mol. Biol., 216:585-610, 1990; Reinhold-Hurek and Shub, Nature, 357:173-176, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence specific cleavage/ligation reactions involving nucleic acids (Joyce, Nature, 338:217-244, 1989; Cech et al., Cell, 27:487-96, 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression (Scanlon et al., Proc Natl Acad. Sci. USA., 88:10591-10595, 1991; Sarver et al., Science, 247:1222-1225, 1990). It has been reported that ribozymes elicited genetic changes in some cell lines to which they are applied; the altered genes included the oncogenes H-ras, c-fos and genes of HIV. Most of this work involved the modification of a target mRNA, based on a specific mutant codon that is cleaved by a specific ribozyme. In light of the information included herein and the knowledge of one of ordinary skill in the art, the preparation and use of ribozymes that are specifically targeted against the NRG1 signaling pathway component gene will be useful in abrogating the function of the NRG1 signaling pathway component gene product.

Methods of Making Transgenic Animals

A transgenic animal can be prepared in a number of ways. A transgenic organism is one that has an extra or exogenous fragment of DNA incorporated into its genome, sometimes replacing an endogenous piece of DNA. At least for the purposes of this invention, any animal whose genome has been modified to introduce a NRG1 signaling pathway component gene mutation, as well as its mutant progeny, are considered transgenic animals. In order to achieve stable inheritance of the extra or exogenous DNA, the integration event must occur in a cell type that can give rise to functional germ cells. The two animal cell types that are used for generating transgenic animals are fertilized egg cells and embryonic stem cells. Embryonic stem (ES) cells can be returned from in vitro culture to a “host” embryo where they become incorporated into the developing animal and can give rise to transgenic cells in all tissues, including germ cells. The ES cells are transfected in culture and then the mutation is transmitted into the germ line by injecting the cells into an embryo. The animals carrying mutated germ cells are then bred to produce transgenic offspring. The use of ES cells to make genetic changed in the mouse germ line is well recognized. For a reviews of this technology, those of skill in the art are referred to Bronson & Smithies, J. Biol. Chem., 269(44), 27155-27158, 1994; Torres, Curr. Top. Dev. Biol., 36, 99-114; 1998 and the references contained therein.

Generally, blastocysts are isolated from pregnant mice at a given stage in development, for example, the blastocyst from mice may be isolated at day 4 of development (where day 1 is defined as the day of plug), into an appropriate buffer that will sustain the ES cells in an undifferentiated, pluripotent state. ES cell lines may be isolated by a number of methods well known to those of skill in the art. For example, the blastocysts may be allowed to attach to the culture dish and approximately 7 days later, the outgrowing inner cell mass picked, trypsinized and transferred to another culture dish in the same culture media. ES cell colonies appear 2-3 weeks later with between 5-7 individual colonies arising from each explanted inner cell mass. The ES cell lines can then be expanded for further analysis. Alternatively, ES cell lines can be isolated using the immunosurgery technique (described in Martin, Proc. Natl. Acad. Sci. USA 78:7634-7638, 1981) where the trophectoderm cells are destroyed using anti-mouse antibodies prior to explanting the inner cell mass.

In generating transgenic animals, the ES cell lines that have been manipulated by homologous recombination are reintroduced into the embryonic environment by blastocyst injection (as described in Williams et al., Cell 52:121-131, 1988). Briefly, blastocysts are isolated from a pregnant mouse and expanded. The expanded blastocysts are maintained in oil-drop cultures at 4° C. for 10 minutes prior to culture. The ES cells are prepared by picking individual colonies, which are then incubated in phosphate-buffered saline, 0.5 mM EGTA for 5 minutes; a single cell suspension is prepared by incubation in a trypsin-EDTA solution containing 1% (v/v) chick serum for a further 5 minutes at 4° C. Five to twenty ES cells (in Dulbecco's modified Eagle's Medium with 10% (v/v) fetal calf serum and 3,000 units/ml DNAase 1 buffered in 20 mM HEPES [pH 8]) are injected into each blastocyst. The blastocysts are then transferred into pseudo-pregnant recipients and allowed to develop normally. The transgenic mice are identified by coat markers (Hogan et al., Manipulating the Mouse Embryo, Cold Spring Harbor, N.Y. (1986)). Additional methods of isolating and propagating ES cells may be found in, for example, U.S. Pat. No. 5,166,065; U.S. Pat. No. 5,449,620; U.S. Pat. No. 5,453,357; U.S. Pat. No. 5,670,372; U.S. Pat. No. 5,753,506; U.S. Pat. No. 5,985,659, each incorporated herein by reference.

An alternative method involving zygote injection method for making transgenic animals is described in, for example, U.S. Pat. No. 4,736,866, incorporated herein by reference. Additional methods for producing transgenic animals are generally described by Wagner and Hoppe (U.S. Pat. No. 4,873,191; which is incorporated herein by reference), Brinster et al. Proc. Nat'l Acad. Sci. USA, 82(13) 4438-4442, 1985; which is incorporated herein by reference in its entirety) and in Manipulating the Mouse Embryo; A Laboratory Manual, 2nd edition (eds., Hogan, Beddington, Costantimi and Long, Cold Spring Harbor Laboratory Press, 1994; which is incorporated herein by reference in its entirety).

Briefly, this method involves injecting DNA into a fertilized egg, or zygote, and then allowing the egg to develop in a pseudo-pregnant mother. The zygote can be obtained using male and female animals of the same strain or from male and female animals of different strains. The transgenic animal that is born, the founder, is bred to produce more animals with the same DNA insertion. In this method of making transgenic animals, the new DNA typically randomly integrates into the genome by a non-homologous recombination event. One to many thousands of copies of the DNA may integrate at a site in the genome Generally, the DNA is injected into one of the pronuclei, usually the larger male pronucleus. The zygotes are then either transferred the same day, or cultured overnight to form 2-cell embryos and then transferred into the oviducts of pseudo-pregnant females. The animals born are screened for the presence of the desired integrated DNA.

DNA clones for microinjection can be prepared by any means known in the art. For example, DNA clones for microinjection can be cleaved with enzymes appropriate for removing the bacterial plasmid sequences, and the DNA fragments electrophoresed on 1% agarose gels in TBE buffer, using standard techniques. The DNA bands are visualized by staining with ethidium bromide, and the band containing the expression sequences is excised. The excised band is then placed in dialysis bags containing 0.3 M sodium acetate, pH 7.0. DNA is electroeluted into the dialysis bags, extracted with a 1:1 phenol:chloroform solution and precipitated by two volumes of ethanol. The DNA is redissolved in 1 ml of low salt buffer (0.2 M NaCl, 20 mM Tris, pH 7.4, and 1 mM EDTA) and purified on an Elutip-DTM column. The column is first primed with 3 ml of high salt buffer (1 M NaCl, 20 mM Tris, pH 7.4, and 1 mM EDTA) followed by washing with 5 ml of low salt buffer. The DNA solutions are passed through the column three times to bind DNA to the column matrix. After one wash with 3 ml of low salt buffer, the DNA is eluted with 0.4 ml high salt buffer and precipitated by two volumes of ethanol. DNA concentrations are measured by absorption at 260 nm in a UV spectrophotometer. For microinjection, DNA concentrations are adjusted to 3 mg/ml in 5 mM Tris, pH 7.4 and 0.1 mM EDTA.

Additional methods for purification of DNA for microinjection are described in Hogan et al. Manipulating the Mouse Embryo (Cold Spring Harbor Laboratory, Cold Spring Harbor, N.Y., 1986), in Palmiter et al Nature 300:611 (1982); in The Qiagenologist, Application Protocols, 3rd edition, published by Qiagen, Inc., Chatsworth, Calif.; and in Sambrook et al. Molecular Cloning: A Laboratory Manual (Cold Spring Harbor Laboratory, Cold Spring Harbor, N.Y., 1989).

In an exemplary microinjection procedure, female mice six weeks of age are induced to superovulate. The superovulating females are placed with males and allowed to mate. After approximately 21 hours, the mated females are sacrificed and embryos are recovered from excised oviducts and placed in an appropriate buffer, e.g., Dulbecco's phosphate buffered saline with 0.5% bovine serum albumin (BSA; Sigma). Surrounding cumulus cells are removed with hyaluronidase (1 mg/ml). Pronuclear embryos are then washed and placed in Earle's balanced salt solution containing 0.5% BSA in a 37.5° C. incubator with a humidified atmosphere at 5% CO₂, 95% air until the time of injection. Embryos can be implanted at the two-cell stage.

Randomly cycling adult female mice are paired with vasectomized males. C57BL/6 or Swiss mice or other comparable strains can be used for this purpose. Recipient females are mated at the same time as donor females. At the time of embryo transfer, the recipient females are anesthetized with an intraperitoneal injection of 0.015 ml of 2.5% avertin per gram of body weight. The oviducts are exposed by a single midline dorsal incision. An incision is then made through the body wall directly over the oviduct. The ovarian bursa is then torn with watchmakers forceps. Embryos to be transferred are placed in DPBS (Dulbecco's phosphate buffered saline) and in the tip of a transfer pipette (about 10 to 12 embryos). The pipette tip is inserted into the infundibulum and the embryos transferred. After the transfer, the incision is closed by two sutures. The pregnant animals then give birth to the founder animals which are used to establish the transgenic line.

Pharmaceutical Compositions

An aspect of the invention involves administering agents to mammals, e.g., administering a test agent to a mammal for the purpose of determining its modulatory activity. In preferred variations of the invention, the test agent is formulated with a pharmaceutically acceptable or a physiological acceptable carrier for administration.

Also, the present invention pertains to pharmaceutical compositions comprising an agent identified by the methods of the invention. The agent can be formulated with a pharmaceutically acceptable or physiologically acceptable carrier or excipient to prepare a pharmaceutical composition. The carrier and composition can be sterile. The formulation should suit the mode of administration.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active agents.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrollidone, sodium saccharine, cellulose and magnesium carbonate.

Methods of introduction of these compositions include, but are not limited to, intradermal, intramuscular, intraperitoneal, intraocular, intravenous, subcutaneous, topical, oral and intranasal. Other suitable methods of introduction can also include gene therapy (as described below), rechargeable or biodegradable devices, particle acceleration devises (“gene guns”) and slow release polymeric devices. The pharmaceutical compositions of this invention can also be administered as part of a combinatorial therapy with other agents.

The composition can be formulated in accordance with the routine procedures as a pharmaceutical composition adapted for administration to human beings. For example, compositions for intravenous administration typically are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

For topical application, nonsprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, sols, liniments, salves, aerosols, etc., which are, if desired, sterilized or mixed with auxiliary agents, e.g., preservatives, stabilizers, wetting agents, buffers or salts for influencing osmotic pressure, etc. The agent may be incorporated into a cosmetic formulation. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., pressurized air.

Agents described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine and procaine.

The agents are administered in a therapeutically effective amount. The amount of agents which will be therapeutically effective in the treatment of a particular disorder or condition, such as a clinical symptom of schizophrenia, will depend on the nature of the disorder or condition, and can be determined by standard clinical techniques. In addition, in vitro or in vivo assays may optionally be employed to help identify optimal dosage ranges. The precise dose to be employed in the formulation will also depend on the route of administration, and the seriousness of the symptoms of schizophrenia, and should be decided according to the judgment of a practitioner and each patient's circumstances. Effective doses may be extrapolated from dose-response curves derived from in vitro or animal model test systems.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use of sale for human administration. The pack or kit can be labeled with information regarding mode of administration, sequence of drug administration (e.g., separately, sequentially or concurrently), or the like. The pack or kit may also include means for reminding the patient to take the therapy. The pack or kit can be a single unit dosage of the combination therapy or it can be a plurality of unit dosages. In particular, the agents can be separated, mixed together in any combination, present in a single vial or tablet. Agents assembled in a blister pack or other dispensing means is preferred. For the purpose of this invention, unit dosage is intended to mean a dosage that is dependent on the individual pharmacodynamics of each agent and administered in FDA approved dosages in standard time courses.

Antibodies

Another aspect of the invention is an antibody that specifically binds polypeptides relevant to the invention. For example, antibody substances that specifically bind component proteins of the NMDA receptor signaling pathway are and protein components of the NRG1 signaling pathway are contemplated, and preferably antibodies that are specific for the phosphorylated or activated state of the polypeptide. Such antibodies can be either monoclonal or polyclonal antibodies, as well fragments thereof and humanized forms or fully human forms, such as those produced in transgenic animals. The invention further provides a hybridoma that produces an antibody according to the invention. Antibodies of the invention are useful for detection and/or purification of the polypeptides of the invention.

Protein may be used to immunize animals to obtain polyclonal and monoclonal antibodies which specifically react with the protein. Such antibodies may be obtained using either the entire protein or fragments thereof as an immunogen. The peptide immunogens additionally may contain a cysteine residue at the carboxyl terminus, and are conjugated to a hapten such as keyhole limpet hemocyanin (KLH). Methods for synthesizing such peptides are known in the art, for example, as in Merrifield, J. Amer. Chem. Soc. 85, 2149-2154, 1963; Krstenansky, et al., FEBS Lett. 211:10, 1987. In general, techniques for preparing polyclonal and monoclonal antibodies as well as hybridomas capable of producing the desired antibody are well known in the art (Campbell, Monoclonal Antibodies Technology: Laboratory Techniques in Biochemistry and Molecular Biology, Elsevier Science Publishers, Amsterdam, The Netherlands, 1984; St. Groth et al., J. Immunol. 35:1-21, 1990; Kohler & Milstein, Nature 256:495-497, 1975), the trioma technique, the human B-cell hybridoma technique (Kozbor et al., Immunology Today 4:72, 1983; Cole et al., in Monoclonal Antibodies and Cancer Therapy, Alan R. Liss, Inc., 1985, pp. 77-96).

Any animal (mouse, rabbit, etc.) which is known to produce antibodies can be immunized with a peptide or polypeptide of the invention. Methods for immunization are well known in the art. Such methods include subcutaneous or intraperitoneal injection of the polypeptide. One skilled in the art will recognize that the amount of the protein or peptide used for immunization will vary based on the animal which is immunized, the antigenicity of the peptide and the site of injection. The protein or peptide that is used as an immunogen may be modified or administered in an adjuvant in order to increase the protein's antigenicity. Methods of increasing the antigenicity of a protein are well known in the art and include, but are not limited to, coupling the antigen with a heterologous protein (such as globulin or galactosidase) or through the inclusion of an adjuvant during immunization.

For monoclonal antibodies, spleen cells from the immunized animals are removed, fused with myeloma cells, such as SP2/0-Ag14 myeloma cells, and allowed to become monoclonal antibody producing hybridoma cells. Any one of a number of methods well known in the art can be used to identify the hybridoma cell which produces an antibody with the desired characteristics. These include screening the hybridomas with an ELISA assay, western blot analysis, or radioimmunoassay (Lutz et al., Exp. Cell Research. 175:109-24, 1988).

Hybridomas secreting the desired antibodies are cloned and the class and subclass is determined using procedures known in the art (Campbell, Monoclonal Antibody Technology: Laboratory Techniques in Biochemistry and Molecular Biology, Elsevier Science Publishers, Amsterdam, The Netherlands (1984)). Techniques described for the production of single chain antibodies (U.S. Pat. No. 4,946,778) can be adapted to produce single chain antibodies to proteins of the present invention.

For polyclonal antibodies, antiserum containing antibodies is isolated from the immunized animal and is screened for the presence of antibodies with the desired specificity using one of the above-described procedures. The present invention further provides the above-described antibodies in delectably labeled form. Antibodies can be delectably labeled through the use of radioisotopes, affinity labels (such as biotin and avidin), enzymatic labels (such as horseradish peroxidase and alkaline phosphatase) fluorescent labels (such as FITC or rhodamine) and paramagnetic atoms. Procedures for accomplishing such labeling are well-known in the art, for example, see Stemberger, et al., J. Histochem. Cytochem. 18:315, 1970; Bayer et al., Meth. Enzym. 62:308, 1979; Engval et al., Immunol. 109:129, 1972; Goding J. Immunol. Meth. 13:215, 1976.

The labeled antibodies of the present invention can be used for in vitro, in vivo, and in situ assays to identify cells or tissues in which a fragment of the polypeptide of interest is expressed. The present invention further provides the above-described antibodies immobilized on a solid support. Examples of such solid supports include plastics such as polycarbonate, complex carbohydrates such as agarose and sepharose, acrylic resins and such as polyacrylamide and latex beads. Techniques for coupling antibodies to such solid supports are well known in the art (Weir et al., Handbook of Experimental Immunology 4th Ed., Blackwell Scientific Publications, Oxford, England, Chapter 10, 1986; Jacoby et al., Meth. Enzym. 34 Academic Press, N.Y., 1974). The immobilized antibodies of the present invention can be used for in vitro, in vivo, and in situ assays as well as for immuno-affinity purification of the proteins of the present invention.

In an alternative embodiment, human antibodies can be produced from phage-display libraries (Hoogenboom et al., J. Mol. Biol. 227:381 (1991); Marks et al., J. Mol. Biol. 222:581 (1991). These processes mimic immune selection through the display of antibody repertoires on the surface of filamentous bacteriophage, and subsequent selection of phage by their binding to an antigen of choice. One such technique is described in PCT Application no. PCT/US98/17364.

Chimeric, CDR grafted, and humanized antibodies are typically produced by recombinant methods. Nucleic acids encoding the antibodies are introduced into host cells and expressed using materials and procedures described herein. In a preferred embodiment, the antibodies are produced in mamma lian host cells, such as CHO cells. Monoclonal (e.g., human) antibodies may be produced by the expression of recombinant DNA in host cells or by expression in hybridoma cells as described herein.

The following examples are for illustration purposes only, and should not be construed as limiting the scope of the invention in any way.

### EXAMPLES

### Example 1

### Analysis of the NRG1 Signaling Cascade

p59Fyn is an ErbB4 Binding Partner

To characterize components of the NRG1 signaling pathway downstream of the ErbB4 receptor tyrosine kinase, the predominant neuronal receptor for NRG1, the cytoplasmic domain of ErbB4 was used as a bait in a yeast two-hybrid interaction screen. The ErbB4 bait was created by cloning the entire cytoplasmic domain into the vector pGBKT7 (Clontech, Palo Alto, Calif.) using homologous recombination in the yeast strain AH109 (Clontech, Palo Alto, Calif.). Liner vector and the cytoplasmic fragment of ErbB4 were transfected into the yeast strain using the lithium acetate (LiAc) method and plated onto leucine negative selection media plates. Plasmid DNA was isolated from positive colonies and the presence of ErbB4 was verified by sequencing.

The ErbB4 bait was capable of tyrosine auto-phosphorylation in yeast cells. Tyrosine phosphorylation was absent from ErbB4 baits which contained mutations in the ATP binding site (K751A) or the catalytic site (D843A). Homo sapiens random prime hippocampus cDNA library was created using the ZAP-cDNA Gigapack III Gold Cloning Kit (Stratagene) and transformed into yeast using Li Acetate. Screening of a human hippocampus cDNA library yielded a partial cDNA clone comprising the SH2 and SH3 domains of Fyn (amino acids 60-252 of SEQ ID NO: 3). SH2 domains are protein modules able to bind phosphorylated tyrosine residues. The interaction was confirmed in a yeast two-hybrid growth assay as well as in yeast β-galactosidase assays. No Fyn clones were isolated when the same cDNA library was screened using ErbB4 K751A as bait, nor were the ErbB4 K751A or D843A baits able to interact with the Fyn clone in the yeast two-hybrid growth assay. These results suggest that Fyn is an ErbB4 binding protein, that ErbB4-Fyn binding is mediated by tyrosine phosphorylation of ErbB4, and that binding is likely through the Fyn SH2 domain.

To determine if full-length ErbB4 and Fyn interact within a mammalian cell, Cos7 cells were transfected to transiently express ErbB4 and Fyn tagged with a C-terminal V5-epitope. The transfected cells were grown on glass slides to 70% confluency, activated with 7.2 nM NRG1 (for 10 minutes, fixed in 4% paraformaldehyde) and permeabilized with 100% methanol for 1 minute. Non-specific binding sites were blocked with 10% FBS in phosphate-buffered saline (PBS) for 1 hour. Primary antibodies (α-ErbB4 antibody (Santa Cruz Biotechnology, Santa Cruz, Calif.) 1:400 and α-V5 antibody (Invitrogen, Carlsbad, Calif.) 1:500) were added overnight at 4° C. Secondary antibodies (Molecular Probes, goat α-rabbit Alexa Fluor-488 and goat α-mouse-Alexa Fluor-594 at 1:500) were added for 4 hours at 4° C. Cells were mounted in PVA-DABCO (Chemicon, Temecula, Calif.) and examined with a Leica deconvolution CTR MIC Microscope using Leica QFluoro Image Manager software. Co-localization of Fyn and ErbB4 in COS7 cells was observed and this co-localization was independent of ErbB4 receptor activation by NRG1, perhaps due to high basal levels of ErbB4 tyrosine phosphorylation in the transfected cells.

Physical association of ErbB4 and Fyn was demonstrated by co-immunoprecipitation from transformed HEK293 cells stably expressing both proteins. The transfected HEK293 cells stably expressing ErbB4 and Fyn-V5 were activated with 7.2 nM NRG1 for 10 minutes and lysed using RIPA-buffer (20 mM MOPS, 0.5 mM EDTA pH 7.0, 150 mM NaCl, 3.5 mM sodium dodecyl sulfate (SDS), 1% NP-40, 1% deoxycholate (DOC)) containing Complete Protease Inhibitor Cocktail (Roche Diagnostics, Basal, Switzerland) and Phosphatase Inhibitor Cocktail Set II (Calbiochem, Temecula, Calif.). 25U of Benzonase Nuclease (Novagen, San Diego, Calif.) per 1 ml of lysate were added to each sample. Lysates were incubated with the appropriate antibodies (1 μg/ml lysate) for 2 hours at 4° C. (α-Glu [Emerald Biostructures], α-ErbB4, α-HA and α-PSD95 [all Santa Cruz]). Immunocomplexes were isolated using Protein G Sepharose 4 Fast Flow (Amersham Biosciences, Uppsula, Sweden) and recovered by boiling for 7 minutes in 1×SDS-PAGE sample buffer. Fyn containing protein complexes were immunoprecipitated using α-ErbB4 (Santa Cruz Biotechnology, Santa Cruz, Calif.) control α-Glu (Emerald Biostructures, Bainbridge Island, Wash.) antibodies.

To demonstrate that ErbB4 tyrosine phosphorylation is required for ErbB4-Fyn interaction, CHO-K1 cells stably co-expressing ErbB4 and Fyn, or co-expressing the kinase-dead mutant ErbB4 D843A and Fyn were used. ErbB4 and Fyn, but not ErbB4 D843A and Fyn co-immunoprecipitation could be demonstrated. These results demonstrate that ErbB4 and Fyn physically interact in mammalian cells, and this interaction may not be solely dependent upon ErbB4 tyrosine phosphorylation.

ErbB4 Binding Partners in Brain

ErbB4 has been shown to be a component of the postsynaptic density in brain, a multi-protein complex containing various protein kinases, including Fyn and other Src-family kinases, the NMDA receptor and scaffolding proteins, such as PSD-95 (Husi et al., Nat. Neurosci. 3: 661-669, 2000). Specific interaction of PSD-95 and ErbB4 also has been demonstrated in brain (Garcia et al., Proc. Natl. Acad. Sci., 97: 3596-3601, 2000).

To further characterize components of the NRG1/ErbB4 signaling pathway a-ErbB4 co-immunoprecipitates from mouse brain hippocampus were analyzed. Animals were euthanized by cervical dislocation as anesthetics were found to alter NR2B Tyr1472 phosphorylation. Hippocampus tissue was isolated by manual dissection and homogenized in DOC buffer (20 μM ZnCl₂, 50 mM Tris pH 9.0, 1% DOC) (1 ml/50 mg of tissue) containing protease and phosphatase inhibitors as above. 25U Benzonase Nuclease per 1 ml of lysate were added to each sample during a 60 minute incubation at 4° C. For SDS-PAGE and Western blot, sample protein content was determined using a BCA Protein Assay Reagent Kit (Pierce, Rockford, Ill.), adjusted to a final concentration of 2 μg/μl total protein in 1×SDS-PAGE sample buffer, and then 20-25 μg of protein was loaded per gel lane. For immunoprecipitation, samples were adjusted to a final concentration of 2 μg/μl total protein in DOC buffer. Lysates were incubated with the appropriate antibodies (1 μg of antibody/250 μg of total protein) overnight at 4° C. (α-Glu, α-ErbB4, α-HA and α-PSD95). Immunocomplexes were isolated using Protein G Sepharose 4 Fast Flow and recovered by boiling for 7 minutes in 1×SDS-PAGE sample buffer. Samples were subjected to one-dimensional SDS-PAGE followed by silver staining, in-gel digestion and mass spectrometric analysis or by Western blotting.

In association with immunoprecipitates of ErbB4, a peptide with a sequence (EIISEVQR; SEQ ID NO: 17) was detected. This peptide matched DDEF2, a Pyk2 binding protein and Src-family kinase substrate. This suggested that Pyk2, a non-receptor tyrosine kinase, that has previously been implicated in the modulation of NMDA receptor function by Huang et al. (Neuron 29: 485-496, 2001), associates with ErbB4. This was confirmed by Western blot analysis of α-ErbB4 co-immunoprecipitates, in which Pyk2 was detected. Pyk2 was also co-immunoprecipitated from mouse brain hippocampus using antibodies directed against PSD-95, confirming a previous observation that PSD-95 and Pyk2 are components of the multi-protein postsynaptic density complex (Husi et al., Neuron 29: 485-496, 2001). No co-immunoprecipitation of Pyk2 was observed when α-HA antibodies were used for immunoprecipitation.

NRG1 Regulation of p59Fyn Kinase

The physical association of Fyn and Pyk2 with ErbB4 suggests that both non-receptor tyrosine kinases may be downstream effectors of NRG1. The initial step of NRG1 signaling is activation of ErbB4 kinase activity and auto-phosphorylation on tyrosine residues. An increase in ErbB4 tyrosine phosphorylation and associated kinase activity was observed when CHO-K1 cells (ECACC Accession No. 85051005) expressing ErbB4 were exposed to NRG1 (recombinant human HRG-αEGF domain; R&D Systems, Minneapolis, Minn.) in serum free medium for 0-15 minutes. Following immunoprecipitation of ErbB4, kinase activity was measured using a peptide substrate. Maximum ErbB4 kinase activity was detected within 1 minute of NRG1 exposure but returned to the pre-stimulation level by 15 minutes.

To determine whether NRG1-induced activation of ErbB4 leads to Fyn phosphorylation, NRG1 exposure of HEK293 cells stably expressing ErbB4 and Fyn-V5 were activated with 7.2 nM NRG1 (R&D Systems; Minneapolis, Minn.) for 10 minutes and lysed using RIPA buffer. Exposure to NRG1 resulted in tyrosine phosphorylation of ErbB4, activation of MAPK p44/42 as shown by an increase in Thr202/Tyr204 phosphorylation, and in phosphorylation of Fyn on Tyr420. There was no change in Fyn Tyr531 phosphorylation. Phosphorylation on Tyr531 holds the kinase in an inactive conformation through interaction with the Fyn SH2 domain (Slater et al., Nat. Rev. Neurosci. 5: 317-328, 2004). Fyn is activated either by dephophorylation of Tyr531 with loss of the SH2 domain interaction, or through displacement of the SH2 domain by a second tyrosine phosphorylated binding partner allowing auto-phosphorylation on the Fyn activation loop residue Tyr420. Alternatively, Fyn can be phosphorylated on Tyr420 and activated directly by a second kinase. Thus, the NRG1-stimulated increase in Fyn Tyr420 phosphorylation may be through stimulation of Fyn auto-phosphorylation, through phosphorylation of Fyn by ErbB4, or through phosphorylation of Fyn by an ErbB4 activated tyrosine kinase other than Fyn.

These alternatives were distinguished by applying PP2 (25 nM; Calbiochem, San Diego, Calif.) a relatively selective Src-family kinase inhibitor, to inhibit Fyn while ErbB4 signaling was stimulated with NRG1. Fyn inhibition is highly sensitive to PP2 with an IC₅₀=5 nM, while PP3, a control kinase inhibitor related to PP2, has no effect on Fyn. Fyn was phosphorylated at a low level on Tyr420 under basal conditions. This was reduced by 24 hour preincubation with PP2, suggesting that Fyn Tyr420 basal phosphorylation was due to autophosphorylation or phosphorylation by another Src-family kinase. After NRG1 stimulation, an increase in Fyn Tyr420 phosphorylation was seen, even under conditions in which PP2 was used to inhibit p59 Fyn autophosphorylation. The control compound PP3 had no effect on Fyn phosphorylation. Thus, ErbB4 or an ErbB4 activated tyrosine kinase may phosphorylate Fyn on Tyr420 in response to NRG1 stimulation and activate the kinase directly.

To test if the observed increase in Fyn Tyr420 phosphorylation was associated with enhanced catalytic activity, Fyn kinase activity was measured after NRG1 stimulation. Fyn kinase activity was increased within 5 minutes of NRG1 stimulation, reached maximum activity after 7 minutes, and returned to pre-stimulation levels within 15 minutes. No increase in Fyn kinase activity was observed when cells co-expressing Fyn and the catalytically inactive ErbB4 D843A mutant were stimulated with NRG1. These results confirm that in mammalian cells, Fyn kinase is activated in response to NRG1 signaling.

p59Fyn Potentiates ErbB4 Mediated NRG1 Signaling

Greater ErbB4 tyrosine phosphorylation was observed in the presence as compared to the absence of Fyn. This suggests that Fyn either directly or indirectly amplifies NRG1 signaling by increasing ErbB4 tyrosine phosphorylation. Thus, NRG1-induced ErbB4 phosphorylation under conditions in which Fyn and related Src-family kinases were inhibited by PP2 (selective Src-family kinase inhibitor described above). PP2 greatly reduced ErbB4 tyrosine phosphorylation, while the control compound PP3 (25 nM; Calbiochem, San Diego, Calif.) had no effect. PP2 had no effect on ErbB4 basal phosphorylation in the absence of NRG1, nor on NRG1 induced tyrosine phosphorylation in HEK293 cells expressing only ErbB4. These results suggest that Fyn is activated in response to NRG1 signaling and that activated Fyn contributes to ErbB4 phosphorylation on tyrosine residues. This may result in the creation of additional SH2 domain docking sites, thus leading to further activation of Fyn as part of a positive feedback mechanism involving NRG1, ErbB4 and Src-family kinases.

Since Fyn kinase activity appeared to contribute to NRG1-induced tyrosine phosphorylation of ErbB4, it was hypothesized that NRG1 dependent signal transduction activity would be enhanced in cells over-expressing Fyn kinase. CHO-K1 cells expressing only ErbB4 were constructed, and subsequently a subclone co-expressing Fyn was identified. The parental ErbB4 expressing cell line and the subclone co-expressing ErbB4 and Fyn continued to express similar levels of ErbB4 as shown by Western blot. The cells also contained a reporter gene sensitive to MAPK pathway activation, the luciferase gene under the control of a promoter containing a serum response element (SRE). The cells were activated for 4 hours with 0-57.6 nM and the luciferase assay was performed using the Dual-Luciferase Reported assay system (Promega, Madison, Wis.) according to the manufacturer's instructions. Induction of the luciferase reporter gene by NRG1 was amplified two-fold (p<0.05) in cells co-expressing ErbB4 and Fyn as compared to cells expressing only ErbB4. No significant differences in baseline luciferase activity or in NRG1 EC₅₀ (˜1 nM) were found between the two cell lines. These results suggest that Fyn amplifies NRG1 signaling through ErbB4.

### Example 2

### In Vitro Analysis of NRG1/ErbB4-Dependent NMDA Receptor Phosphorylation

Fyn and Pyk2 previously have been implicated in the phosphorylation of the NR2A and NR2B subunits of the NMDA receptor (Tezuka et al., Proc. Natl. Acad. Sci. 96: 435-440, 1999; Nakazawa et al., J. Biol. Chem. 276: 693-699, 2001; Heid et al., J. Neurosci. 22: 5452-5461, 2002). It also has been reported that NR2B Tyr1472 phosphorylation is increased after induction of long term potentiation (LTP) in the hippocampal CA1 region (Nakazawa et al., J. Biol. Chem. 276: 693-699), and that Pyk2 is required for LTP induction (Yang et al., J. Neurosci. 23: 4072-4080, 2003).

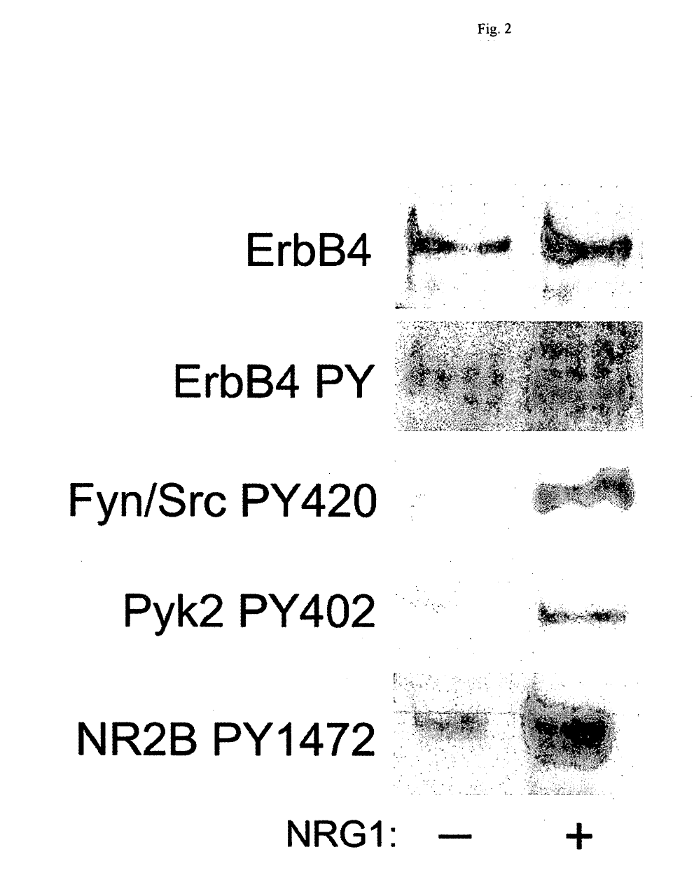

Studies were carried out to determine if NRG1 signaling, mediated by activation of Src-family kinases, results in changes in the phosphorylation state of regulatory tyrosine residues on NR2B. BE(2)-M 17 human neuroblastoma cells (ECACC No. 95011816) have a neuron-like appearance after differentiation for 72 hours in the presence of 10 μM retinoic acid (RA; Sigma, St. Louis, Mo.) (Hanada et al., Can. Res. 53: 4978-4986, 1993). Western blot analysis indicated that BE(2)-M17 cells expressed ErbB4, Fyn, Src, Pyk2, PSD95 and NR1. As RA differentiation induces NR2B, but not NR2A, expression, it was expected that the functional NMDA receptor channels expressed in these cells comprised of NR1 and NR2B subunits. Stimulation of differentiated BE(2)-M17 cells with NRG1 for 10 minutes increased ErbB4 tyrosine phosphorylation (FIG. 2), increased Fyn/Src Tyr420 phosphorylation (indicative of increased Src-family kinase activity), and increased Pyk2 Tyr402 phosphorylation. Pyk2 Tyr402 is an auto-phosphorylation site involved in Src-family kinase recruitment (Dikic et al., Nature 383: 547-550, 1996). In addition, a coincident increase in NR2B Tyr1472 phosphorylation was also observed (FIG. 2). Thus, NRG1 signaling through ErbB4 modulates NR2B tyrosine phosphorylation in neuroblastoma cells.

### Example 3

### In Vivo Analysis of NMDA Phosphorylation for Identification of Anti-Schizophrenic Drugs

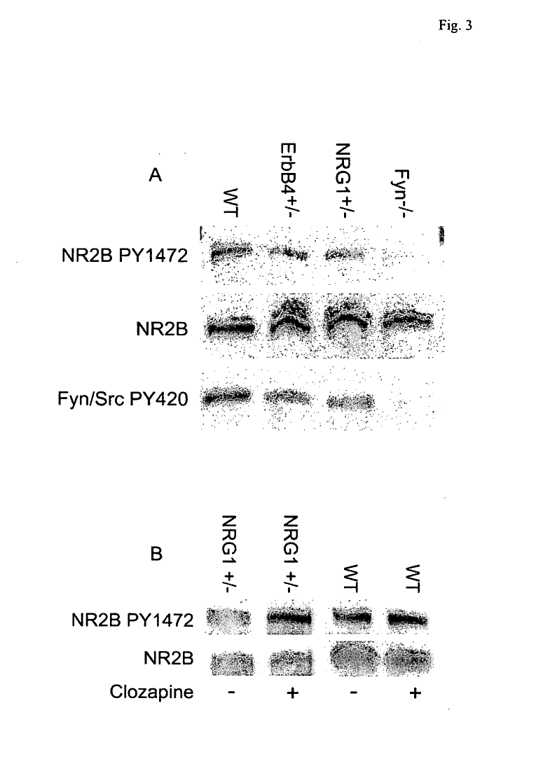

It was hypothesized that NRG1^{+/-} and ErbB4^{+/-} mutant mice might show altered regulation of NMDA receptor subunit NR2B Tyr1472 phosphorylation related to their abnormal behavioral phenotype. Hippocampal lysates were analyzed as described in Example 1. It was determined that Tyr1472 of NR2B was hypo-phosphorylated in hippocampal lysates from 9 month old NRG1^{+/-} or ErbB4^{+/-} mutant mice as compared to age and sex matched C57B1/6 control mice (FIG. 3). There was no apparent difference in total NR2B protein comparing any of the mutant and control mice. As reported by others, NR2B Tyr1472 phosphorylation is also reduced in Fyn^{-/-} null mice (Grant et al., Science 258: 1903-1910, 1992). Tyr1472 phosphorylation in NRG1^{+/-} or ErbB4^{+/-} mutant mice was reduced to the level seen in Fyn^{-/-} null mice implying that NRG1 is a regulator of Fyn-mediated NR2B tyrosine phosphorylation. Fyn/Src Tyr420 phosphorylation also was reduced in the mutant mice compared to the controls, suggesting attenuated Src-family kinase activity in an environment of altered NRG1 signaling. Although the α-Fyn/Src PY420 antibody reagent (1:800; Biosource, Camarillo, Calif.) used for Western blotting did not distinguish between Fyn and Src, the signal is virtually absent in Fyn^{-/-} null mice suggesting that Fyn is the predominant kinase in the hippocampal lysate. These results suggest that in animals deficient for components of the NRG1 signaling pathway, altered regulation of NR2B Tyr1472 phosphorylation plays a role in NMDA receptor modulation.

Behavioral abnormality of NRG1^{+/-} mutant mice observed in the open field test is reversible with clozapine at a dose of 1 mg/kg i.p. The activity level of wild-type litter mate controls is not affected by that dose of clozapine. Thus, clozapine is not simply sedating at 1 mg/kg i.p., but has a selective effect on the behavioral abnormality of NRG1^{+/-} mutant mice. Reversal of the behavioral abnormality occurred rapidly within 25 minutes of a single exposure to clozapine.

To determine if clozapine at the same dose reversed the hypophosphorylation of NR2B, clozapine was administered to age and sex matched NRG1^{+/-} mice and C57B1/6 wild-type controls, and hippocampal lysates were prepared 25 minutes after injection. NR2B Tyr1472 phosphorylation was increased 2.5-3 fold when normalized against total NR2B (FIG. 3). NR2B Tyr1472 phosphorylation in clozapine-treated NRG1 ^{+/-} mice was indistinguishable from age and sex matched controls. The same dose of clozapine had no effect on NR2B Tyr1472 phosphorylation when administered to wild-type mice. Thus, reversal of the behavioral abnormality of hyperactivity in the open field in NRG1^{+/-} mutant mice is associated with restoration of NR2B Tyr1472 phosphorylation to normal levels.

### Example 4

### Methods of the Invention Identified Potential Modulator of NMDA Receptor Function

The cell-based methods of identifying modulators of NMDA receptor function described herein identified a small molecule (denoted herein as D150838), identified by proprietary methods, that induces tyrosine phosphorylation of the NMDA receptor subunit NR2B. CHO cells expressing ErbB4 polypeptide were contacted with D150838 to determine whether D150838 activated the NRG1 signaling pathway. The activation of the NRG1 signaling pathway was measured by immunoprecipitating ErbB4 polypeptide and detecting tyrosine phosphorylated ErbB4 by Western blot analysis with an antibody specific for phosphotyrosine residues. The addition of D150838 to CHO cells induced tyrosine phosphorylation of ErbB4 at a higher level than phosphorylation induced by exposure to NRG1 (positive control). Control assays using other tyrosine kinase receptors (PDGF receptor and insulin receptor) that are tyrosine phosphorylated upon activation were also carried out. D150838 did not induce tyrosine phosphorylation of either the PDGF receptor or the insulin receptor. The control assays demonstrate that D150838 acts specifically on the NRG1 signaling pathway.

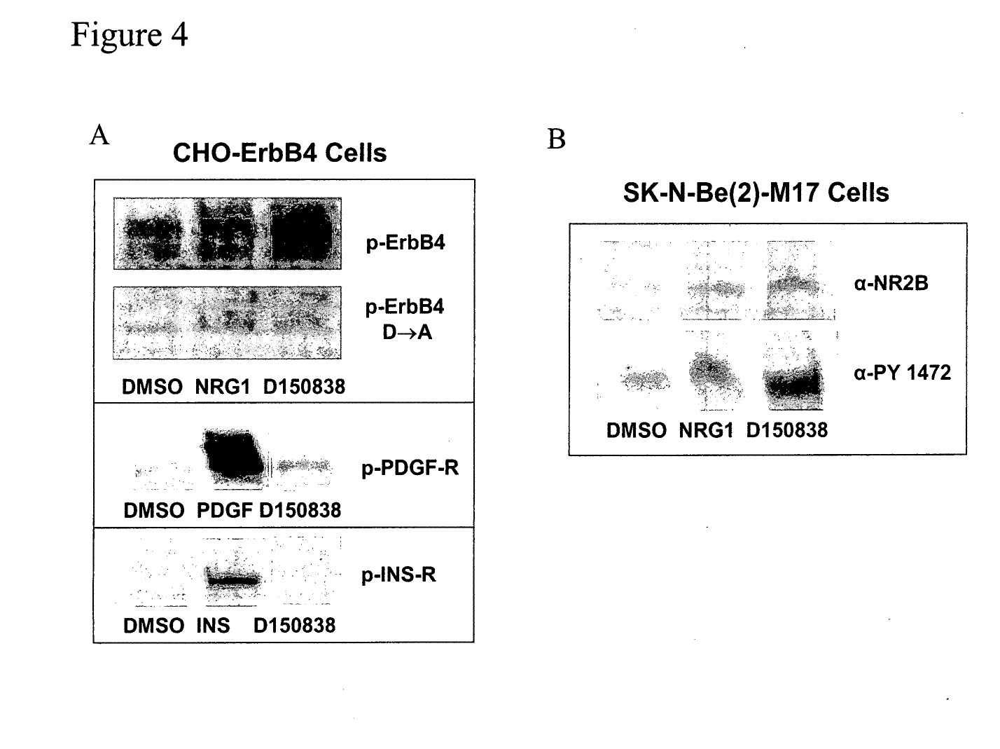

To determine if D150838 modulates tyrosine phosphorylation of the NMDA receptor, SK-N-BE(2)-M17 cells were grown for 72 house in the presence of retinoic acid to induce expression of the NMDA receptor NR2B subunit. The cells were contacted with NRG1 or D150838, the NR2B subunit was immunoprecipitated, and the level of tyrosine phosphorylation was detected by Western blot analysis with an anti-phosphotyrosine antibody. Addition of NRG1 or D150838 induced tyrosine phosphorylation of the NR2B subunit. Phosphorylation of tyrosine residue 1472 was detected after exposure to D150838. As depicted in FIG. 4, these experiments demonstrate that methods of the present invention have successfully identified a modulator of NMDA receptor function as measured by phosphorylation of the tyrosine residue 1472.

What is claimed:

1. A method of identifying a modulator of N-methyl-D-Aspartate (NMDA) receptor function comprising
(a) administering a test agent to a mammal harboring a genetic defect in a NRG1 signaling pathway, wherein the mammal has an NMDA receptor complex hypophosphorylation phenotype;
(b) measuring tyrosine phosphorylation of NMDA receptor complex in the brain of the mammal after administering the test agent; and
(c) identifying a modulator of NMDA receptor function from the measurement of NMDA receptor complex tyrosine phosphorylation.

2. The method of claim 1 wherein the measuring tyrosine phosphorylation of the NMDA receptor complex comprises measuring phosphorylation of a NMDA receptor.

3. The method of claim 1 wherein the measuring tyrosine phosphorylation of the NMDA receptor complex comprises measuring phosphorylation of a NMDA receptor subunit NR2A

4. The method of claim 1 wherein the measuring tyrosine phosphorylation of the NMDA receptor complex comprises measuring phosphorylation of a NMDA receptor subunit NR2B.

5. The method of claim 4 wherein the measuring tyrosine phosphorylation of the NMDA receptor complex comprises measuring phosphorylation of a tyrosine corresponding to the tyrosine at position 1472 of the NMDA receptor subunit NR2B amino acid sequence of SEQ ID NO: 6.

6. The method of claim 1 wherein step (c) comprises:
comparing the measurement of NMDA receptor complex tyrosine phosphorylation in the brain of the mammal administered the test agent with a measurement of NMDA receptor complex tyrosine phosphorylation in the brain of a control mammal that did not receive the test agent,
wherein the control mammal has the genetic defect and the NMDA receptor complex hypophosphorylation phenotype, and
wherein a difference in NMDA receptor complex tyrosine phosphorylation measurements between the mammal administered the test agent and the control mammal identifies the test agent as a modulator of NMDA receptor function.

7. The method of claim 6, wherein a greater NMDA receptor complex tyrosine phosphorylation measurement in the mammal administered the test agent identifies the test agent as a positive modulator of NMDA receptor function.

8. The method of claim 1 wherein step (c) comprises:
comparing the measurement of NMDA receptor complex tyrosine phosphorylation in the brain of the mammal administered the test agent with a measurement of NMDA receptor complex tyrosine phosphorylation in the brain of a control mammal free of the genetic defect and having a wild type NMDA receptor complex phosphorylation phenotype,
wherein similar NMDA receptor complex tyrosine phosphorylation measurements in the mammal harboring the genetic defect and the control mammal identifies the test agent as a positive modulator of NMDA receptor function.

9. The method of claim 1, wherein the administering step comprises administering two or more concentrations of the test agent to two or more of the mammals, wherein dose-dependent differences in NMDA receptor complex tyrosine phosphorylation measurements from in the mammals identifies the test agent as a modulator of NMDA receptor function.

10. The method of claim 1 wherein step (c) comprises:
comparing the measurement of NMDA receptor complex tyrosine phosphorylation in the brain of the mammal administered the test agent with a measurement of NMDA receptor complex tyrosine phosphorylation in the brain of a control mammal that received an agent known to increases NMDA receptor complex tyrosine phosphorylation,
wherein the control mammal has the genetic defect and the NMDA receptor complex hypophosphorylation phenotype, and
wherein similar NMDA receptor complex tyrosine phosphorylation measurements between the mammal administered the test agent and the control mammal identifies the test agent as a positive modulator of NMDA receptor function.

11. A method of identifying a modulator of N-methyl-D-aspartate (NMDA) receptor function, comprising
(a)(1) administering an agent that reduces phosphorylation of an NMDA receptor complex to a mammal, in an amount effective to induce an NMDA receptor complex hypophosphorylation phenotype in the mammal;
(a)(2) administering a test agent to the mammal;
(b) measuring tyrosine phosphorylation of a NMDA receptor complex in the brain of the mammal after administering the test agent; and
(c) identifying a modulator of NMDA receptor function from the measurement of tyrosine phosphorylation.

12. The method of claim 11 wherein the measuring tyrosine phosphorylation of the NMDA receptor complex comprises measuring phosphorylation of a NMDA receptor.

13. The method of claim 11 wherein the measuring tyrosine phosphorylation of the NMDA receptor complex comprises measuring phosphorylation of a NMDA receptor subunit NR2A

14. The method of claim 11 wherein the measuring tyrosine phosphorylation of the NMDA receptor complex comprises measuring phosphorylation of a NMDA receptor subunit NR2B.

15. The method of claim 14 wherein the measuring tyrosine phosphorylation of the NMDA receptor complex comprises measuring phosphorylation of a tyrosine corresponding to the tyrosine at position 1472 of the NMDA receptor subunit NR2B amino acid sequence of SEQ ID NO: 6.

16. The method of claim 11 wherein step (c) comprises:
comparing the measurement of NMDA receptor complex tyrosine phosphorylation in the brain of the mammal administered the test agent with a measurement of NMDA receptor complex tyrosine phosphorylation in the brain of a control mammal that did not receive the test agent,
wherein the control mammal has the agent-induced NMDA receptor complex hypophosphorylation phenotype, and
wherein a difference in NMDA receptor complex tyrosine phosphorylation measurements between the mammal administered the test agent and the control mammal identifies the test agent as a modulator of NMDA receptor function.

17. The method of claim 11, wherein a greater NMDA receptor complex tyrosine phosphorylation measurement in the mammal administered the test agent identifies the test agent as a positive modulator of NMDA receptor function.

18. The method of claim 11 wherein step (c) comprises:
comparing the measurement of NMDA receptor complex tyrosine phosphorylation in the brain of the mammal administered the test agent with a measurement of NMDA receptor complex tyrosine phosphorylation in the brain of a control mammal free of the agent-induced NMDA receptor complex hypophosphorylated phenotype and having a wild type NMDA receptor complex phosphorylation phenotype,
wherein similar NMDA receptor complex tyrosine phosphorylation measurements in the mammal receiving the phosphorylation-reducing agent and the control mammal identifies the test agent as an positive modulator of NMDA receptor function.

19. The method of claim 11, wherein the administering step comprises administering two or more concentrations of the test agent to two or more of the mammals with the agent-induced NMDA receptor complex hypophosphorylation phenotype, wherein dose-dependent differences in NMDA receptor complex tyrosine phosphorylation measurements in the mammals identifies the test agent as a modulator of NMDA receptor function.

20. The method of claim 11, wherein the agent that reduces phosphorylation of the NMDA receptor complex is phencyclidine (PCP), ketamine or amphetamine.

21. A method of identifying modulators of N-methyl-D-aspartate (NMDA) receptor function comprising
(a) administering a test agent to a mammal exhibiting a behavioral symptom of schizophrenia, wherein the mammal has a NRG1 signaling pathway protein hypophosphorylation phenotype;
(b) measuring tyrosine phosphorylation of a NRG1 signaling pathway protein in the brain of the mammal after administering the test agent; and
(c) identifying a modulator of NMDA receptor function from the measurement of tyrosine phosphorylation of the NRG1 signaling pathway protein.

22. The method of claim 21, wherein the NRG1 signaling pathway protein is selected from the group consisting of ErbB4, Fyn or Pyk2.

23. The method of claim 21 wherein step (c) comprises comparing the measurement of tyrosine phosphorylation of a positive regulatory site of the NRG1 signaling pathway protein in the brain of the mammal administered the test agent with a measurement of tyrosine phosphorylation of the NRG1 signaling pathway protein in the brain of a control mammal not administered the test agent, wherein the control mammal exhibits the same behavioral symptom, and wherein a test agent that increases tyrosine phosphorylation of a positive regulatory site of the NRG1 signaling pathway protein in the mammal administered the test agent is identified as a positive modulator of NMDA receptor function.

24. The method of claim 21 wherein step (c) comprises comparing the measurement of tyrosine phosphorylation of the NRG1 signaling pathway protein in the brain of the mammal administered the test agent with a measurement of tyrosine phosphorylation of the NRG1 signaling pathway protein in the brain of a wild type mammal that does not exhibit the symptom, wherein a test agent that increases the tyrosine phosphorylation of a positive regulatory site of a NRG1 signaling pathway protein in the mammal having the NRG1 signaling pathway protein hypophosphorylation phenotype to a level similar to the level of tyrosine phosphorylation of the NRG1 signaling pathway protein in the brain of the wild type mammal is identified to be a positive modulator of NMDA receptor function.

25. The method of claim 21 wherein the behavioral symptom of schizophrenia is selected from the group consisting of hyperactivity, deceased social interaction, defective memory function, defective cognitive function, decreased pre-pulse inhibition and combinations thereof.

26. A method of identifying modulators of N-methyl-D-aspartate (NMDA) receptor function comprising
(a) administering a test agent to a mammal exhibiting a behavioral symptom of schizophrenia, wherein the mammal has a NMDA receptor complex hypophosphorylation phenotype;
(b) measuring tyrosine phosphorylation of a NMDA receptor complex in the brain of the mammal after administering the test agent; and
(c) identifying a modulator of NMDA receptor function from the measurement of tyrosine phosphorylation of the NMDA receptor complex.

27. The method of claim 26 wherein the measuring tyrosine phosphorylation of the NMDA receptor complex comprises measuring phosphorylation of a NMDA receptor.

28. The method of claim 26 wherein the measuring tyrosine phosphorylation of the NMDA receptor complex comprises measuring phosphorylation of a NMDA receptor subunit NR2A.

29. The method of claim 26 wherein the measuring tyrosine phosphorylation of the NMDA receptor complex comprises measuring phosphorylation of a NMDA receptor subunit NR2B.

30. The method of claim 26 wherein the measuring tyrosine phosphorylation of the NMDA receptor complex comprises measuring phosphorylation of a tyrosine corresponding to the tyrosine at position 1472 of the NMDA receptor subunit NR2B amino acid sequence of SEQ ID NO: 6.

31. The method of claim 26 wherein step (c) comprises comparing the measurement of tyrosine phosphorylation of the NMDA receptor complex in the brain of the mammal administered the test agent with a measurement of tyrosine phosphorylation of the NMDA receptor complex in the brain of a control mammal not administered the test agent, wherein the control mammal exhibits the same behavioral symptom, and wherein a test agent that increases tyrosine phosphorylation in the mammal administered the test agent is identified as a positive modulator of NMDA receptor function.

32. The method of claim 26 wherein step (c) comprises comparing the measurement of tyrosine phosphorylation of the NMDA receptor complex in the brain of the mammal administered the test agent with a measurement of tyrosine phosphorylation of the NMDA receptor complex in the brain of a wild type mammal that does not exhibit the symptom, wherein a test agent that increases the tyrosine phosphorylation in the mammal administered the test agent to a level similar to the level of tyrosine phosphorylation in the brain of the wild type mammal is identified to be a positive modulator of NMDA receptor function.

33. The method of claim 26 wherein the behavioral symptom of schizophrenia is selected from the group consisting of hyperactivity, decreased social interaction, defective memory function, defective cognitive function, decreased pre-pulse inhibition and combinations thereof.

34. The method according to any one of claims 1, 11, 21 or 26, wherein the mammal is a non-human primate.

35. The method according to any one of claims 1, 11, 21 or 26, wherein the mammal is a rodent.

36. The method according to any one of claims 1, 11, 21 or 26, wherein the mammal is selected from the group consisting of NRG1^{-/-} mice, NRG1 ^{+/-} mice, ErbB4^{+/-} mice and ErbB4^{-/-} mice.

37. The method according to any one of claims 1, 11, 21 or 26, wherein the mammal is a rodent and wherein the genetic defect is within a gene encoding a NRG1 signaling pathway protein selected from the group consisting of NRG1, ErbB4, Fyn and Pyk2.

38. The method according to any one of claims 1, 11, 21 or 26, wherein the mammal is a rodent selected from the group consisting of NMDA receptor subunit NR1^{-/-} mice, Reeler mice, DiGeorge mice, proline-dehydrogenase mutant mice and 5HT-2A receptor^{-/-} mice.

39. A method according to any one of claims 1, 11, 21 or 26, wherein the administering comprises a route selected from the group consisting of intradermal administration, intramuscular administration, inttaperitoneal administration, intraocular administration, subcutaneous administration, topical administration, oral administration and intranasal administration.

40. A method according to any one of claims 1, 11, 21 or 26, wherein the test agent is formulated in a carrier selected from the group consisting of water, saline, salt solutions, buffered saline, alcohol, glycerol, ethanol, gum arabic, vegetable oils, benzyl alcohols, polyethylene glycols, gelatin, carbohydrates, dextrose, magnesium stearate, talc, silicic acid, viscous paraffin, perfume oil, fatty esters, hydroxymethylcellulose, polyvinyl pyroline and a combination thereof.

41. A method according to any one of claims 1, 11, 21 or 26, wherein the measuring comprises an immunoassay using an antibody substance that recognizes a phosphorylated tyrosine amino acid.

42. A method according to any one of claims 1, 11, 21 or 26, further comprising steps of monitoring the mammal that receives the test agent for behavioral symptoms of schizophrenia.

43. A method according to any one of claims 1, 11, 21 or 26, further comprising:
(d) formulating a composition comprising a pharmaceutically acceptable carrier and a test agent identified in step (c) as a modulator of NMDA receptor function;
(e) administering the composition to a mammal that exhibits behavioral symptoms of schizophrenia; and
(f) monitoring the mammal for a decrease in the behavioral characteristics of schizophrenia.

44. A method of screening for a candidate compound to palliate schizophrenia comprising
(a) contacting a test agent to a cell that expresses a NMDA receptor subunit NR2 and
(b) measuring tyrosine phosphorylation of the NR2 subunit of the NMDA receptor of the cell in the presence and absence of the test agent, wherein an increase in tyrosine phosphorylation of the NMDA receptor NR2 subunit in the presence of the test agent identifies an agent as a candidate compound to palliate schizophrenia.

45. The method of claim 44 wherein the NR2 subunit is NR2A or NR2B.

46. The method of claim 44 wherein the measuring comprises measuring phosphorylation of a tyrosine corresponding to the tyrosine at position 1472 of the NMDA receptor subunit NR2B amino-acid sequence set forth in SEQ ID NO: 6.

47. The method of claim 44, wherein the cell is recombinantly modified to express increased amounts of NMDA receptor subunit NR2.

48. The method of claim 44 wherein the cell further expresses a NRG1 signaling pathway protein.

49. The method of claim 48 wherein the NRG1 signaling pathway protein is ErbB4, Fyn or Pyk2.

50. The method of claim 44, wherein the cell is a mammalian cell.

51. The method of claim 44, wherein the cell is a neuron or a cell from a neuronal cell line.

52. The method of claim 51, wherein the cell is selected from the group consisting of a BE(2)-M17 cell (ECACC No. 95011816) or SH-SY5Y (ATCC Accession No. CRL-2266).

53. A method according to claim 44, further comprising:
(d) formulating a composition comprising a pharmaceutically acceptable carrier and a test agent identified in step (c) as a modulator of NMDA receptor function;
(e) administering the composition to a mammal that exhibits behavioral symptoms of schizophrenia; and
(f) monitoring the mammal for a decrease in the behavioral characteristics of schizophrenia.

55. A method of identifying modulators of N-methyl-D-aspartate (NMDA) receptor function comprising
(a) contacting a test agent to a cell that expresses a NMDA receptor subunit NR2, ErbB4 and Fyn; and
(b) measuring tyrosine phosphorylation of a NRG1 signaling pathway protein in the presence and absence of the test agent, wherein an increase in tyrosine phosphorylation of a positive regulatory site of the NRG1 signaling pathway protein the presence of the test agent identifies an agent as a positive modulator of NMDA receptor function, and wherein a decrease in tyrosine phosphorylation of a negative regulatory site of the NRG1 signaling pathway component in the presence of the test agent identifies an agent as a negative modulator of NMDA receptor function.

55. The methods of claim 44 or 54 wherein the cell of the contacting step is transfected with a polynucleotide that encodes at least one protein of the NRG1 signaling pathway.

56. The method of claim 55 wherein the cell is transfected with a polynucleotide that encodes at least one of the NRG1 signaling pathway proteins selected from the group consisting of
(a) ErbB4 polypeptide having the amino acid sequence of SEQ ID NO: 12,
(b) Fyn polypeptide having the sequence of SEQ ID NO: 14, and
(c) Pyk2 polypeptide having the sequence of SEQ ID NO: 16.

57. The method of claim 44 or 54 wherein the cell is transfected with a polynucleotide encoding at least one NMDA receptor subunit NR2 selected from the group consisting of
(a) NR2A polypeptide having an amino acid sequence encoded by the polynucleotide sequence of SEQ ID NO: 4, and
(b) NR2B polypeptide having the sequence of SEQ ID NO: 6.

58. A method of identifying a modulator of N-methyl-D-aspartate (NMDA) receptor function, comprising:
(a) culturing a cell in the presence and absence of a test agent, wherein the cell expresses NR2 polypeptide and polypeptide components of a NRG1 signaling pathway, and wherein the cell exhibits a physiological or biochemical response to neuregulin 1 (NRG1), and
(b) measuring tyrosine phosphorylation of a NRG1 signaling pathway protein in the presence and absence of the test agent, wherein an increase in tyrosine phosphorylation of a positive regulatory site of the NGR1 signaling pathway protein in the presence of the test agent identifies the agent as a positive modulator of NMDA receptor function, and wherein a decrease in tyrosine phosphorylation of a negative regulatory site of the NRG1 signaling pathway protein in the presence of the test agent identifies and agent that is a negative modulator of NMDA receptor function.


SEQUENCE LISTING (18 sequences)

SEQ ID NO: 1 - DNA, 4663 bp, Homo sapiens
ggtaaggctctggaaaagggggcgctgggagcgcattgcgagggggctggagagggagagaggagcggaagctgagggtgtgaaacggctggccccgaacacacctcgcggcgctccagtgattcctggtgtccgacctcagccccagtcagtgcgggtccagtttccaggctctcgcggaaggcctggctgagcacatgcggcagccacggtcgccctccctattcctcttagcccgaggaggggggtcccaagttacatggccacgcagatggggcctctccctcatttctgaaccttgtggggaggggaaccttgaagggagcgccccccagagccatggcttagggcctcccccacccctctggagctccagtctgcaagagtcaggagccgaaatatcgctgactgtgggtgacgactcttgcgcgcacacacacatacaagcgggcacgacgcgttcggtcctattaaaaggcacgcaagggtgcggctgcacgcggtgacacggacccctctaacgtttccaaactgagctccctgcaggtccccgacagcacaggcccctgtcccaggacccctccaggcacgcgctcacacgcacacgcgcgctccccggctcacgcgcgctccgacacacacgctcacgcgaacgcaggcgcacgctctggcgcgggaggcgccccttcgcctccgtgttgggaagcgggggcggcgggaggggcaggagacgttggccccgctcgcgtttctgcagctgctgcagtcgccgcagcgtccggaccggaaccagcgccgtccgcggagccgccgccgccgccgccgggccctttccaagccgggcgctcggagctgtgcccggccccgcttcagcaccgcggacagcgccggccgcgtggggctgagccgagcccccgcgcacgcttcagcgccccttccctcggccgacgtcccgggaccgccgctccgggggagacgtggcgtccgcagcccgcggggccgggcgagcgcaggacggcccggaagccccgcgggggatgcgccgagggccccgcgttcgcgccgcgcagagccaggcccgcggcccgagcccatgagcaccatgcgcctgctgacgctcgccctgctgttctcctgctccgtcgcccgtgccgcgtgcgaccccaagatcgtcaacattggcgcggtgctgagcacgcggaagcacgagcagatgttccgcgaggccgtgaaccaggccaacaagcggcacggctcctggaagattcagctcaatgccacctccgtcacgcacaagcccaacgccatccagatggctctgtcggtgtgcgaggacctcatctccagccaggtctacgccatcctagttagccatccacctacccccaacgaccacttcactcccacccctgtctcctacacagccggcttctaccgcatacccgtgctggggctgaccacccgcatgtccatctactcggacaagagcatccacctgagcttcctgcgcaccgtgccgccctactcccaccagtccagcgtgtggtttgagatgatgcgtgtctacagctggaaccacatcatcctgctggtcagcgacgaccacgagggccgggcggctcagaaacgcctggagacgctgctggaggagcgtgagtccaaggcagagaaggtgctgcagtttgacccagggaccaagaacgtgacggccctgctgatggaggcgaaagagctggaggcccgggtcatcatcctttctgccagcgaggacgatgctgccactgtataccgcgcagccgcgatgctgaacatgacgggctccgggtacgtgtggctggtcggcgagcgcgagatctcggggaacgccctgcgctacgccccagacggcatcctcgggctgcagctcatcaacggcaagaacgagtcggcccacatcagcgacgccgtgggcgtggtggcccaggccgtgcacgagctcctcgagaaggagaacatcaccgacccgccgcggggctgcgtgggcaacaccaacatctggaagaccgggccgctcttcaagagagtgctgatgtcttccaagtatgcggatggggtgactggtcgcgtggagttcaatgaggatggggaccggaagttcgccaactacagcatcatgaacctgcagaaccgcaagctggtgcaagtgggcatctacaatggcacccacgtcatccctaatgacaggaagatcatctggccaggcggagagacagagaagcctcgagggtaccagatgtccaccagactgaagattgtgacgatccaccaggagcccttcgtgtacgtcaagcccacgctgagtgatgggacatgcaaggaggagttcacagtcaacggcgacccagtcaagaaggtgatctgcaccgggcccaacgacacgtcgccgggcagcccccgccacacggtgcctcagtgttgctacggcttttgcatcgacctgctcatcaagctggcacggaccatgaacttcacctacgaggtgcacctggtggcagatggcaagttcggcacacaggagcgggtgaacaacagcaacaagaaggagtggaatgggatgatgggcgagctgctcagcgggcaggcagacatgatcgtggcgccgctaaccataaacaacgagcgcgcgcagtacatcgagttttccaagcccttcaagtaccagggcctgactattctggtcaagaaggagattccccggagcacgctggactcgttcatgcagccgttccagagcacactgtggctgctggtggggctgtcggtgcacgtggtggccgtgatgctgtacctgctggaccgcttcagccccttcggccggttcaaggtgaacagcgaggaggaggaggaggacgcactgaccctgtcctcggccatgtggttctcctggggcgtcctgctcaactccggcatcggggaaggcgcccccagaagcttctcagcgcgcatcctgggcatggtgtgggccggctttgccatgatcatcgtggcctcctacaccgccaacctggcggccttcctggtgctggaccggccggaggagcgcatcacgggcatcaacgaccctcggctgaggaacccctcggacaagtttatctacgccacggtgaagcagagctccgtggatatctacttccggcgccaggtggagctgagcaccatgtaccggcatatggagaagcacaactacgagagtgcggcggaggccatccaggccgtgagagacaacaagctgcatgccttcatctgggactcggcggtgctggagttcgaggcctcgcagaagtgcgacctggtgacgactggagagctgtttttccgctcgggcttcggcataggcatgcgcaaagacagcccctggaagcagaacgtctccctgtccatcctcaagtcccacgagaatggcttcatggaagacctggacaagacgtgggttcggtatcaggaatgtgactcgcgcagcaacgcccctgcgacccttacttttgagaacatggccggggtcttcatgctggtagctgggggcatcgtggccgggatcttcctgattttcatcgagattgcctacaagcggcacaaggatgctcgccggaagcagatgcagctggcctttgccgccgttaacgtgtggcggaagaacctgcagcagtaccatcccactgatatcacgggcccgctcaacctctcagatccctcggtcagcaccgtggtgtgaggcccccggaggcgcccacctgcccagttagcccggccaaggacactgatgggtcctgctgctcgggaaggcctgagggaagcccacccgccccagagactgcccaccctgggcctcccgtccgtccgcccgcccaccccgctgcctggcgggcagcccctgctggaccaaggtgcggaccggagcggctgaggacggggcagagctgagtcggctgggcagggcgcagggcgctccggcagaggcagggccctggggtctctgagcagtggggagcgggggctaactggccccaggcgaaggggcttggagcagagacggcagccccatccttcccgcagcaccagcctgagccacagtggggcccatggccccagctggctgggtcgcccctcctcgggcgcctgcgctcctctgcagcctgagctccaccctcccctcttcttgcggcaccgcccacccacaccccgtctgccccttgaccccacacgccggggctggccctgccctcccccacggccgtccctgacttcccagctggcagcgcctcccgccgcctcgggccgcctcctccagactcgagagggctgagcccctcctctcctcgtccggcctgcagcccagaacgggcctccccgggggtccccggacgctggctcgggactgtcttcaaccctgccctgcaccttgggcacgggagagcgccacccgcccgcccccgccctcgctccgggtgcgtgaccggcccgccaccttgtacagaaccagcactcccagggcccgagcgcgtgccttccccgtgcggcccgtgcgcagccgcgctctgcccctccgtccccagggtgcaggcgcgcaccgcccaacccccacctcccggtgtatgcagtggtgatgcctaaaggaatgtcacgc

SEQ ID NO: 2 - protein, 885 aa, Homo sapiens
MSTMRLLTLALLFSCSVARAACDPKIVNIGAVLSTRKHEQMFREAVNQANKRHGSWKIQLNATSVTHKPNAIQMALSVCEDLISSQVYAILVSHPPTPNDHFTPTPVSYTAGFYRIPVLGLTTRMSIYSDKSIHLSFLRTVPPYSHQSSVWFEMMRVYSWNHIILLVSDDHEGRAAQKRLETLLEERESKAEKVLQFDPGTKNVTALLMEAKELEARVIILSASEDDAATVYRAAAMLNMTGSGYVWLVGEREISGNALRYAPDGILGLQLINGKNESAHISDAVGVVAQAVHELLEKENITDPPRGCVGNTNIWKTGPLFKRVLMSSKYADGVTGRVEFNEDGDRKFANYSIMNLQNRKLVQVGIYNGTHVIPNDRKIIWPGGETEKPRGYQMSTRLKIVTIHQEPFVYVKPTLSDGTCKEEFTVNGDPVKKVICTGPNDTSPGSPRHTVPQCCYGFCIDLLIKLARTMNFTYEVHLVADGKFGTQERVNNSNKKEWNGMMGELLSGQADMIVAPLTINNERAQYIEFSKPFKYQGLTILVKKEIPRSTLDSFMQPFQSTLWLLVGLSVHVVAVMLYLLDRFSPFGRFKVNSEEEEEDALTLSSAMWFSWGVLLNSGIGEGAPRSFSARILGMVWAGFAMIIVASYTANLAAFLVLDRPEERITGINDPRLRNPSDKFIYATVKQSSVDIYFRRQVELSTMYRHMEKHNYESAAEAIQAVRDNKLHAFIWDSAVLEFEASQKCDLVTTGELFFRSGFGIGMRKDSPWKQNVSLSILKSHENGFMEDLDKTWVRYQECDSRSNAPATLTFENMAGVFMLVAGGIVAGIFLIFIEIAYKRHKDARRKQMQLAFAAVNVWRKNLQQYHPTDITGPLNLSDPSVSTVV

SEQ ID NO: 3 - DNA, 6293 bp, Homo sapiens
atcatgggaccgggtgagcgctgagaatcgcggccgcagccatcagccctggagatgaccaggagcggccactgctgagaactatgtggagagaggctgcgagccctgctgcagagcctccggctgggatagccgccccccgtgggggcgatgcggacagcgcgggacagccaggggagcgcgctggggccgcagcatgcgggaacccgctaaacccggtggctgctgaggcggccgagatgctcgtgcgcgcagcgcgccccactgcatcctcgaccttctcgggctacagggaccgtcagtggcgactatgggcagagtgggctattggaccctgctggtgctgccggcccttctggtctggcgcggtccggcgccgagcgcggcggcggagaagggtccccccgcgctaaatattgcggtgatgctgggtcacagccacgacgtgacagagcgcgaacttcgaacactgtggggccccgagcaggcggcggggctgcccctggacgtgaacgtggtagctctgctgatgaaccgcaccgaccccaagagcctcatcacgcacgtgtgcgacctcatgtccggggcacgcatccacggcctcgtgtttggggacgacacggaccaggaggccgtagcccagatgctggattttatctcctcccacaccttcgtccccatcttgggcattcatgggggcgcatctatgatcatggctgacaaggatccgacgtctaccttcttccagtttggagcgtccatccagcagcaagccacggtcatgctgaagatcatgcaggattatgactggcatgtcttctccctggtgaccactatcttccctggctacagggaattcatcagcttcgtcaagaccacagtggacaacagctttgtgggctgggacatgcagaatgtgatcacactggacacttcctttgaggatgcaaagacacaagtccagctgaagaagatccactcttctgtcatcttgctctactgttccaaagacgaggctgttctcattctgagtgaggcccgctcccttggcctcaccgggtatgatttcttctggattgtccccagcttggtctctgggaacacggagctcatcccaaaagagtttccatcgggactcatttctgtctcctacgatgactgggactacagcctggaggcgagagtgagggacggcattggcatcctaaccaccgctgcatcttctatgctggagaagttctcctacatccccgaggccaaggccagctgctacgggcagatggagaggccagaggtcccgatgcacaccttgcacccatttatggtcaatgttacatgggatggcaaagacttatccttcactgaggaaggctaccaggtgcaccccaggctggtggtgattgtgctgaacaaagaccgggaatgggaaaaggtgggcaagtgggagaaccatacgctgagcctgaggcacgccgtgtggcccaggtacaagtccttctccgactgtgagccggatgacaaccatctcagcatcgtcaccctggaggaggccccattcgtcatcgtggaagacatagaccccctgaccgagacgtgtgtgaggaacaccgtgccatgtcggaagttcgtcaaaatcaacaattcaaccaatgaggggatgaatgtgaagaaatgctgcaaggggttctgcattgatattctgaagaagctttccagaactgtgaagtttacttacgacctctatctggtgaccaatgggaagcatggcaagaaagttaacaatgtgtggaatggaatgatcggtgaagtggtctatcaacgggcagtcatggcagttggctcgctcaccatcaatgaggaacgttctgaagtggtggacttctctgtgccctttgtggaaacgggaatcagtgtcatggtttcaagaagtaatggcaccgtctcaccttctgcttttctagaaccattcagcgcctctgtctgggtgatgatgtttgtgatgctgctcattgtttctgccatagctgtttttgtctttgaatacttcagccctgttggatacaacagaaacttagccaaagggaaagcaccccatgggccttcttttacaattggaaaagctatatggcttctttggggcctggtgttcaataactccgtgcctgtccagaatcctaaagggaccaccagcaagatcatggtatctgtatgggccttcttcgctgtcatattcctggctagctacacagccaatctggctgccttcatgatccaagaggaatttgtggaccaagtgaccggcctcagtgacaaaaagtttcagagacctcatgactattccccaccttttcgatttgggacagtgcctaatggaagcacggagagaaacattcggaataactatccctacatgcatcagtacatgaccaaatttaatcagaaaggagtagaggacgccttggtcagcctgaaaacggggaagctggacgctttcatctacgatgccgcagtcttgaattacaaggctgggagggatgaaggctgcaagctggtgaccatcgggagtgggtacatctttgccaccaccggttatggaattgcccttcagaaaggctctccttggaagaggcagatcgacctggccttgcttcagtttgtgggtgatggtgagatggaggagctggagaccctgtggctcactgggatctgccacaacgagaagaacgaggtgatgagcagccagctggacattgacaacatggcgggcgtattctacatgctggctgccgccatggcccttagcctcatcaccttcatctgggagcacctcttctactggaagctgcgcttctgtttcacgggcgtgtgctccgaccggcctgggttgctcttctccatcagcaggggcatctacagctgcattcatggagtgcacattgaagaaaagaagaagtctccagacttcaatctgacgggatcccagagcaacatgttaaaactcctccggtcagccaaaaacatttccagcatgtccaacatgaactcctcaagaatggactcacccaaaagagctgctgacttcatccaaagaggttccctcatcatggacatggtttcagataaggggaatttgatgtactcagacaacaggtcctttcaggggaaagagagcatttttggagacaacatgaacgaactccaaacatttgtggccaaccggcagaaggataacctcaataactatgtattccagggacaacatcctcttactctcaatgagtccaaccctaacacggtggaggtggccgtgagcacagaatccaaagcgaactctagaccccggcagctgtggaagaaatccgtggattccatacgccaggattcactatcccagaatccagtctcccagagggatgaggcaacagcagagaataggacccactccctaaagagccctaggtatcttccagaagagatggcccactctgacatttcagaaacgtcaaatcgggccacgtgccacagggaacctgacaacagtaagaaccacaaaaccaaggacaactttaaaaggtcagtggcctccaaataccccaaggactgtagtgaggtcgagcgcacctacctgaaaaccaaatcaagctcccctagagacaagatctacactatagatggtgagaaggagcctggtttccacttagatccaccccagtttgttgaaaatgtgaccctgcccgagaacgtggacttcccggacccctaccaggatcccagtgaaaacttccgcaagggggactccacgctgccaatgaaccggaaccccttgcataatgaagaggggctttccaacaacgaccagtataaactctactccaagcacttcaccttgaaagacaagggttccccgcacagtgagaccagcgagcgataccggcagaactccacgcactgcagaagctgcctttccaacatgcccacctattcaggccacttcaccatgaggtcccccttcaagtgcgatgcctgcctgcggatggggaacctctatgacatcgatgaagaccagatgcttcaggagacaggtaacccagccaccggggagcaggtctaccagcaggactgggcacagaacaatgcccttcaattacaaaagaacaagctaaggattagccgtcagcattcctacgataacattgtcgacaaacctagggagctagaccttagcaggccctcccggagcataagcctcaaggacagggaacggcttctggagggaaatttttacggcagcctgtttagtgtcccctcaagcaaactctcggggaaaaaaagctcccttttcccccaaggtctggaggacagcaagaggagcaagtctctcttgccagaccacacctccgataaccctttcctccactcccacagggatgaccaacgcttggttattgggagatgcccctcggacccttacaaacactcgttgccatcccaggcggtgaatgacagctatcttcggtcgtccttgaggtcaacggcatcgtactgttccagggacagtcggggccacaatgatgtgtatatttcggagcatgttatgccttatgctgcaaataagaataatatgtactctacccccagggttttaaattcctgcagcaatagacgcgtgtacaagaaaatgcctagtatcgaatctgatgtttaaaaatcttccattaatgttttatctatagggaaatacacgtaatggccaatgttctggagggtaaatgttggatgtccaatagtgccctgctaagaggaagaagatgtagggaggtattttgttgttgttgttgttggctcttttgcacacggcttcatgccataatcttccactcaaggaatcttgtgaggtgtgtgctgagcatggcagacaccagataggtgagtccttaaccaaaaataactaactacataagggcaagtctccgggacatgcctactgggtatgttggcaataatgatgcattggatgccaatggtgatgttatgatttcctatattccaaattccattaaggtcagcccaccatgtaattttctcatcagaaatgcctaatggtttctctaatacagaataagcaatatggtgtgcatgtaaacctgacacagacaaaataaaaacagttaagaatgcatctgcactgtagtcggatttgaacatgtgcaagagattaggaagtttggctcgtaacagtttcagctttcttgttatgccttccatcacagcccaggctcaccccaagaactccaggctcccctaaagaatagcaaatcagtgtgttcgtgatgactgtgctaccttcattatagttcatttccaagacacatctggagccaaaggcccgagggaccctcaggtggggagagctacaggaatctctttggatgttgatgtgtgtttctctctaccctcggcttcgatggtcttgttcagagctgcataaactaacacatttatgtctccgagatctaagtgtggatcttctgtctgtgacacagtggccattgtagtttatcccgaagacgcctatgtacgtaagtttgcatttcctcccttctggtgatgactcagggttgtatagtatctgttaccccttccctcccagagtaaccataactcgttccgtttccaaacagccatggtggtgtccaattagctgtgtatcgctcttcccagagttgttaatgtggtgacatgcaccaacagccgtatgtgtactgtgatctgtaagaagtacaatgccatctgtctgccgaaggctagcatggttttaggtttatcttccttcacatccagaaattctgttggacactcacttccaccccaaactcctcaaatcaaaagccttcaaaacacgaggcactcttggatctaccctgagtatcctccaaactgtggatacagtttagtgagacaagcaatttctcccttctgagttattctctctgttggtggcaaaccacttcatagcaccaacagagatgtaggaaaaattcctcaaagtatttgtcatttctgagtcgcctgcattatcccattcttattctcctcaaacctgtgcatatatgacatgaaatgatatccattttttttttaagttagaaacagagaggggaatacttatgcatggggagcctgttagcacagtgcctgccacaaaaacaagtgcccccgacaagatagttgctatgttatgacactttctcagatcaggattttctagtttaaaaattaaatatcataaaacg

SEQ ID NO: 4 - protein, 1464 aa, Homo sapiens [sequence omitted]

SEQ ID NO: 5 - DNA, 6240 bp, Homo sapiens
ttgaatttgcatctcttcaagacacaagattaaaacaaaatttacgctaaattggattttaaattatcttccgttcatttatccttcgtctttcttatgtggatatgcaagcgagaagaagggactggacattcccaacatgctcactcccttaatctgtccgtctagaggtttggcttctacaaaccaagggagtcgacgagttgaagatgaagcccagagcggagtgctgttctcccaagttctggttggtgttggccgtcctggccgtgtcaggcagcagagctcgttctcagaagagcccccccagcattggcattgctgtcatcctcgtgggcacttccgacgaggtggccatcaaggatgcccacgagaaagatgatttccaccatctctccgtggtaccccgggtggaactggtagccatgaatgagaccgacccaaagagcatcatcacccgcatctgtgatctcatgtctgaccggaagatccagggggtggtgtttgctgatgacacagaccaggaagccatcgcccagatcctcgatttcatttcagcacagactctcaccccgatcctgggcatccacgggggctcctctatgataatggcagataaggatgaatcctccatgttcttccagtttggcccatcaattgaacagcaagcttccgtaatgctcaacatcatggaagaatatgactggtacatcttttctatcgtcaccacctatttccctggctaccaggactttgtaaacaagatccgcagcaccattgagaatagctttgtgggctgggagctagaggaggtcctcctactggacatgtccctggacgatggagattctaagatccagaatcagctcaagaaacttcaaagccccatcattcttctttactgtaccaaggaagaagccacctacatctttgaagtggccaactcagtagggctgactggctatggctacacgtggatcgtgcccagtctggtggcaggggatacagacacagtgcctgcggagttccccactgggctcatctctgtatcatatgatgaatgggactatggcctccccgccagagtgagagatggaattgccataatcaccactgctgcttctgacatgctgtctgagcacagcttcatccctgagcccaaaagcagttgttacaacacccacgagaagagaatctaccagtccaatatgctaaataggtatctgatcaatgtcacttttgaggggaggaatttgtccttcagtgaagatggctaccagatgcacccgaaactggtgataattcttctgaacaaggagaggaagtgggaaagggtggggaagtggaaagacaagtccctgcagatgaagtactatgtgtggccccgaatgtgtccagagactgaagagcaggaggatgaccatctgagcattgtgaccctggaggaggcaccatttgtcattgtggaaagtgtggaccctctgagtggaacctgcatgaggaacacagtcccctgccaaaaacgcatagtcactgagaataaaacagacgaggagccgggttacatcaaaaaatgctgcaaggggttctgtattgacatccttaagaaaatttctaaatctgtgaagttcacctatgacctttacctggttaccaatggcaagcatgggaagaaaatcaatggaacctggaatggtatgattggagaggtggtcatgaagagggcctacatggcagtgggctcactcaccatcaatgaggaacgatcggaggtggtcgacttctctgtgcccttcatagagacaggcatcagtgtcatggtgtcacgcagcaatgggactgtctcaccttctgccttcttagagccattcagcgctgacgtatgggtgatgatgtttgtgatgctgctcatcgtctcagccgtggctgtctttgtctttgagtacttcagccctgtgggttataacaggtgcctcgctgatggcagagagcctggtggaccctctttcaccatcggcaaagctatttggttgctctggggtctggtgtttaacaactccgtacctgtgcagaacccaaaggggaccacctccaagatcatggtgtcagtgtgggccttctttgctgtcatcttcctggccagctacactgccaacttagctgccttcatgatccaagaggaatatgtggaccaggtttctggcctgagcgacaaaaagttccagagacctaatgacttctcaccccctttccgctttgggaccgtgcccaacggcagcacagagagaaatattcgcaataactatgcagaaatgcatgcctacatgggaaagttcaaccagaggggtgtagatgatgcattgctctccctgaaaacagggaaactggatgccttcatctatgatgcagcagtgctgaactatatggcaggcagagatgaaggctgcaagctggtgaccattggcagtgggaaggtctttgcttccactggctatggcattgccatccaaaaagattctgggtggaagcgccaggtggaccttgctatcctgcagctctttggagatggggagatggaagaactggaagctctctggctcactggcatttgtcacaatgagaagaatgaggtcatgagcagccagctggacattgacaacatggcaggggtcttctacatgttgggggcggccatggctctcagcctcatcaccttcatctgcgaacaccttttctattggcagttccgacattgctttatgggtgtctgttctggcaagcctggcatggtcttctccatcagcagaggtatctacagctgcatccatggggtggcgatcgaggagcgccagtctgtaatgaactcccccaccgcaaccatgaacaacacacactccaacatcctgcgcctgctgcgcacggccaagaacatggctaacctgtctggtgtgaatggctcaccgcagagcgccctggacttcatccgacgggagtcatccgtctatgacatctcagagcaccgccgcagcttcacgcattctgactgcaaatcctacaacaacccgccctgtgaggagaacctcttcagtgactacatcagtgaggtagagagaacgttcgggaacctgcagctgaaggacagcaacgtgtaccaagatcactaccaccatcaccaccggccccatagtattggcagtgccagctccatcgatgggctctacgactgtgacaacccacccttcaccacccagtccaggtccatcagcaagaagcccctggacatcggcctcccctcctccaagcacagccagctcagtgacctgtacggcaaattctccttcaagagcgaccgctacagtggccacgacgacttgatccgctccgatgtctctgacatctcaacccacaccgtcacctatgggaacatcgagggcaatgccgccaagaggcgtaagcagcaatataaggacagcctgaagaagcggcctgcctcggccaagtcccgcagggagtttgacgagatcgagctggcctaccgtcgccgaccgccccgctcccctgaccacaagcgctacttcagggacaaggaagggctacgggacttctacctggaccagttccgaacaaaggagaactcaccccactgggagcacgtagacctgaccgacatctacaaggagcggagtgatgactttaagcgcgactccatcagcggaggagggccctgtaccaacaggtctcacatcaagcacgggacgggcgacaaacacggcgtggtcagcggggtacctgcaccttgggagaagaacctgaccaacgtggagtgggaggaccggtccgggggcaacttctgccgcagctgtccctccaagctgcacaactactccacgacggtgacgggtcagaactcgggcaggcaggcgtgcatccggtgtgaggcttgcaagaaagcaggcaacctgtatgacatcagtgaggacaactccctgcaggaactggaccagccggctgccccagtggcggtgacgtcaaacgcctccaccactaagtaccctcagagcccgactaattccaaggcccagaagaagaaccggaacaaactgcgccggcagcactcctacgacaccttcgtggacctgcagaaggaagaagccgccctggccccgcgcagcgtaagcctgaaagacaagggccgattcatggatgggagcccctacgcccacatgtttgagatgtcagctggcgagagcacctttgccaacaacaagtcctcagtgcccactgccggacatcaccaccacaacaaccccggcggcgggtacatgctcagcaagtcgctctaccctgaccgggtcacgcaaaaccctttcatccccacttttggggacgaccagtgcttgctccatggcagcaaatcctacttcttcaggcagcccacggtggcgggggcgtcgaaagccaggccggacttccgggcccttgtcaccaacaagccggtggtctcggcccttcatggggccgtgccagcccgtttccagaaggacatctgtatagggaaccagtccaacccctgtgtgcctaacaacaaaaaccccagggctttcaatggctccagcaatgggcatgtttatgagaaactttctagtattgagtctgatgtctgagtgagggaacagagaggttaaggtgggtacgggagggtaaggctgtgggtcgcgtgatgcgcatgtcacggagggtgacgggggtgaacttggttcccatttgctcctttcttgttttaatttatttatgggatcctggagttctggttcctactgggggcaaccctggtgaccagcaccatctctcctccttttcacagttctctccttcttccccccgctgtcagccattcctgttcccatgagatgatgccatgggccctctcagcaggggagggtagagcggagaaaggaagggctgcatgcgggcttcctcctggtgtggaagagctccttgatatcctctttgagtgaagctgggagaaccaaaaagaggctatgtgagcacaaaggtagcttttcccaaactgatcttttcatttaggtgaggaagcaaaagcatctatgtgagaccatttagcacactgcttgtgaaaggaaagaggctctggctaaattcatgctgcttagatgacatctgtctaggaatcatgtgccaagcagaggttgggaggccatttgtgtttatatataagcccaaaaatgcttgcttcaaccccatgagactcgatagtggtggtgaacagaacccaaggtcattggtggcagagtggattcttgaacaaactggaaagtacgttatgatagtgtcccccggtgccttggggacaagagcaggtggattgtgcgtgcatgtgtgttcatgcacacttgcacccatgtgtagtcaggtgcctcaagagaaggcaaccttgactcttttattgtttctttcaatatccccaagcagtgtgattgtttggcttatatacagacagagatggccatgtattacctgaattttggctgtgtctcccttcatccttctggaataaggagaatgaaaattcttgataaagaagattctgtggtctaaacaaaaaaaggcggtgagcaatcctgcaagagcaaggtacataaacaagtcctcagtggttggcaactgtttcaacttgtttgaaccaagaaccttccaggaaggctaaagggaaaccgaatttcacagccatgattcttttgcccacacttgggacgaaaagattctacaaagctcttttgagcatttagactctcgactggccaaggtttggggaagaacgaacggacctttgaagaagtaaggagtcgtgtatggtagggtaagtgagagagggggatgtttcctatgctttgatcccttctcacttaacctgaagctagacgagcaggcttcttccccccaaaactgattacaactgctacagagcagacagttaagagaaatgagcttgacatttaagagaaatgagctgcactccatgagtgcagctctggaggtacgaaaagaggggaagagacttggaaatgggagacgggggcagagagggaccctccaccacctctttgggcctggctgggtgggaatgtgacttgagcccagagtgaacactcttggtagaagcccttctaccttcctgcaacacctgttccctctcagattgtaccattgagccggaac

SEQ ID NO: 6 - protein, 1484 aa, Homo sapiens
MKPRAECCSPKFWLVLAVLAVSGSRARSQKSPPSIGIAVILVGTSDEVAIKDAHEKDDFHHLSVVPRVELVAMNETDPKSIITRICDLMSDRKIQGVVFADDTDQEAIAQILDFISAQTLTPILGIHGGSSMIMADKDESSMFFQFGPSIEQQASVMLNIMEEYDWYIFSIVTTYFPGYQDFVNKIRSTIENSFVGWELEEVLLLDMSLDDGDSKIQNQLKKLQSPIILLYCTKEEATYIFEVANSVGLTGYGYTWIVPSLVAGDTDTVPAEFPTGLISVSYDEWDYGLPARVRDGIAIITTAASDMLSEHSFIPEPKSSCYNTHEKRIYQSNMLNRYLINVTFEGRNLSFSEDGYQMHPKLVIILLNKERKWERVGKWKDKSLQMKYYVWPRMCPETEEQEDDHLSIVTLEEAPFVIVESVDPLSGTCMRNTVPCQKRIVTENKTDEEPGYIKKCCKGFCIDILKKISKSVKFTYDLYLVTNGKHGKKINGTWNGMIGEVVMKRAYMAVGSLTINEERSEVVDFSVPFIETGISVMVSRSNGTVSPSAFLEPFSADVWVMMFVMLLIVSAVAVFVFEYFSPVGYNRCLADGREPGGPSFTIGKAIWLLWGLVFNNSVPVQNPKGTTSKIMVSVWAFFAVIFLASYTANLAAFMIQEEYVDQVSGLSDKKFQRPNDFSPPFRFGTVPNGSTERNIRNNYAEMHAYMGKFNQRGVDDALLSLKTGKLDAFIYDAAVLNYMAGRDEGCKLVTIGSGKVFASTGYGIAIQKDSGWKRQVDLAILQLFGDGEMEELEALWLTGICHNEKNEVMSSQLDIDNMAGVFYMLGAAMALSLITFICEHLFYWQFRHCFMGVCSGKPGMVFSISRGIYSCIHGVAIEERQSVMNSPTATMNNTHSNILRLLRTAKNMANLSGVNGSPQSALDFIRRESSVYDISEHRRSFTHSDCKSYNNPPCEENLFSDYISEVERTFGNLQLKDSNVYQDHYHHHHRPHSIGSASSIDGLYDCDNPPFTTQSRSISKKPLDIGLPSSKHSQLSDLYGKFSFKSDRYSGHDDLIRSDVSDISTHTVTYGNIEGNAAKRRKQQYKDSLKKRPASAKSRREFDEIELAYRRRPPRSPDHKRYFRDKEGLRDFYLDQFRTKENSPHWEHVDLTDIYKERSDDFKRDSISGGGPCTNRSHIKHGTGDKHGVVSGVPAPWEKNLTNVEWEDRSGGNFCRSCPSKLHNYSTTVTGQNSGRQACIRCEACKKAGNLYDISEDNSLQELDQPAAPVAVTSNASTTKYPQSPTNSKAQKKNRNKLRRQHSYDTFVDLQKEEAALAPRSVSLKDKGRFMDGSPYAHMFEMSAGESTFANNKSSVPTAGHHHHNNPGGGYMLSKSLYPDRVTQNPFIPTFGDDQCLLHGSKSYFFRQPTVAGASKARPDFRALVTNKPVVSALHGAVPARFQKDICIGNQSNPCVPNNKNPRAFNGSSNGHVYEKLSSIESDV

SEQ ID NO: 7 - DNA, 4339 bp, Homo sapiens
cccttaataagatttgccacgtacactcgagccatcgcgagtgtccttgagccgcgggtgacggtggctctcgctgctcgcgccccctcctcccgcggggggagcctgatgccacgttccctatgaattatttatcgccggcctaaaaataccccgaacttcacagcccgagtgaccctccggtggacatgggtggggccctggggccggccctgttgctcacctcgctcttcggtgcctgggcagggctgggtccggggcagggcgagcagggcatgacggtggccgtggtgtttagcagctcagggccgccccaggcccagttccgtgcccgcctcaccccccagagcttcctggacctacccctggagatccagccgctcacagttggggtcaacaccaccaaccccagcagcctcctcacccagatctgcggcctcctgggtgctgcccacgtccacggcattgtctttgaggacaacgtggacaccgaggcggtggcccagatccttgacttcatctcctcccagacccatgtgcccatcctcagcatcagcggaggctctgctgtggtcctcacccccaaggagccgggctccgccttcctgcagctgggcgtgtccctggagcagcagctgcaggtgctgttcaaggtgctggaagagtacgactggagcgccttcgccgtcatcaccagcctgcacccgggccacgcgctcttcctggagggcgtgcgcgccgtcgccgacgccagccacgtgagttggcggctgctggacgtggtcacgctggagctgggcccgggagggccgcgcgcgcgcacgcagcgcctgctgcgccagctcgacgcgcccgtgtttgtggcctactgctcgcgcgaggaggccgaggtgctcttcgccgaggcggcgcaggccggtctggtggggcccggccacgtgtggctggtgcccaacctggcgctgggcagcaccgatgcgccccccgccaccttccccgtgggcctcatcagcgtcgtcaccgagagctggcgcctcagcctgcgccagaaggtgcgcgacggcgtggccattctggccctgggcgcccacagctactggcgccagcatggaaccctgccagccccggccggggactgccgtgttcaccctgggcccgtcagccctgcccgggaggccttctacaggcacctactgaatgtcacctgggagggccgagacttctccttcagccctggtgggtacctggtccagcccaccatggtggtgatcgccctcaaccggcaccgcctctgggagatggtggggcgctgggagcatggcgtcctatacatgaagtaccccgtgtggcctcgctacagtgcctctctgcagcctgtggtggacagtcggcacctgacggtggccacgctggaagagcggccctttgtcatcgtggagagccctgaccctggcacaggaggctgtgtccccaacaccgtgccctgccgcaggcagagcaaccacaccttcagcagcggggacgtggccccctacaccaagctctgctgtaagggattctgcatcgacatcctcaagaagctggccagagtggtcaaattctcctacgacctgtacctggtgaccaacggcaagcatggcaagcgggtgcgcggcgtatggaacggcatgattggggaggtgtactacaagcgggcagacatggccatcggctccctcaccatcaatgaggaacgctccgagatcgtagacttctctgtaccctttgtggagacgggcatcagtgtgatggtggctcgcagcaatggcaccgtctccccctcggccttcttggagccatatagccctgcagtgtgggtgatgatgtttgtcatgtgcctcactgtggtggccatcaccgtcttcatgttcgagtacttcagccctgtcagctacaaccagaacctcaccagaggcaagaagtccgggggcccagctttcactatcggcaagtccgtgtggctgctgtgggcgctggtcttcaacaactcagtgcccatcgagaacccgcggggcaccaccagcaagatcatggttctggtctgggccttctttgctgtcatcttcctcgccagctacacggccaacctggccgccttcatgatccaagagcaatacatcgacactgtgtcgggcctcagtgacaagaagtttcagcggcctcaagatcagtacccacctttccgcttcggcacggtgcccaacggcagcacggagcggaacatccgcagtaactaccgtgacatgcacacccacatggtcaagttcaaccagcgctcggtggaggacgcgctcaccagcctcaagatggggaagctggatgccttcatctatgatgctgctgtcctcaactacatggcaggcaaggacgagggctgcaagctggtcaccattgggtctggcaaggtctttgctaccactggctacggcatcgccatgcagaaggactcccactggaagcgggccatagacctggcgctcttgcagttcctgggggacggagagacacagaaactggagacagtgtggctctcagggatctgccagaatgagaagaacgaggtgatgagcagcaagctggacatcgacaacatggcaggcgtcttctacatgctgctggtggccatggggctggccctgctggtcttcgcctgggagcacctggtctactggaagctgcgccactcggtgcccaactcatcccagctggacttcctgctggctttcagcaggggcatctacagctgcttcagcggggtgcagagcctcgccagcccaccgcggcaggccagcccggacctcacggccagctcggcccaggccagcgtgctcaagatgctgcaggcagcccgcgacatggtgaccacggcgggcgtaagcagctccctggaccgcgccactcgcaccatcgagaattggggtggcggccgccgtgcgcccccaccgtccccctgcccgaccccgcggtctggccccagcccatgcctgcccacccccgacccgcccccagagccgagccccacgggctggggaccgccagacgggggtcgcgcggcgcttgtgcgcagggctccgcagcccccgggccgccccccgacgccggggccgcccctgtccgacgtctcccgagtgtcgcgccgcccagcctgggaggcgcggtggccggtgcggaccgggcactgcgggaggcacctctcggcctccgagcggcccctgtcgcccgcgcgctgtcactacagctcctttcctcgagccgaccgatccggccgccccttcctcccgctcttcccggagcccccggagctggaggacctgccgctgctcggtccggagcagctggcccggcgggaggccctgctgcacgcggcctgggcccggggctcgcgcccgcgtcacgcttccctgcccagctccgtggccgaggccttcgctcggcccagctcgctgcccgctgggtgcaccggccccgcctgcgcccgccccgacggccactcggcctgcaggcgcttggcgcaggcgcagtcgatgtgcttgccgatctaccgggaggcctgccaggagggcgagcaggcaggggcccccgcctggcagcacagacagcacgtctgcctgcacgcccacgcccacctgccattttgctggggggctgtctgtcctcaccttccaccctgtgccagccacggctcctggctctccggggcctgggggcctctggggcacaggggcaggactctggggctgggcacaggctacagagacagtgggggactggacgagatcagcagtgtagcccgtgggacgcaaggcttcccgggaccctgcacctggagacggatctccagtctggagtcagaagtgtgagttatcagccactcaggctccgagccagctggattctctgcctgccactgtcagggttaagcggcaggcaggattgggcttttctggcttctaccatgaaatcctggccatgggaccccagtgacagatgatgtcttccatggtcatcagtgacctcagtagcctcaaatcatggtgagggctgggcttttgctgtcctcttctcacgcagagttctgccaggagggtgtgctgtgggggtcagactcctgaggctctcccttccctggggctagccagttactggtcatgcctgctgtgggcatggaggctggaacttgtggttgaggcagggccatcccgatccttgctctacctggctagagtttcttctcatcagagcactgggacattaaacccaccttttcccagaaaaaaaaaaaaaaaaaaaaaaaaaaaaa

SEQ ID NO: 8 - protein, 1236 aa, Homo sapiens
MGGALGPALLLTSLFGAWAGLGPGQGEQGMTVAVVFSSSGPPQAQFRARLTPQSFLDLPLEIQPLTVGVNTTNPSSLLTQICGLLGAAHVHGIVFEDNVDTEAVAQILDFISSQTHVPILSISGGSAVVLTPKEPGSAFLQLGVSLEQQLQVLFKVLEEYDWSAFAVITSLHPGHALFLEGVRAVADASHVSWRLLDVVTLELGPGGPRARTQRLLRQLDAPVFVAYCSREEAEVLFAEAAQAGLVGPGHVWLVPNLALGSTDAPPATFPVGLISVVTESWRLSLRQKVRDGVAILALGAHSYWRQHGTLPAPAGDCRVHPGPVSPAREAFYRHLLNVTWEGRDFSFSPGGYLVQPTMVVIALNRHRLWEMVGRWEHGVLYMKYPVWPRYSASLQPVVDSRHLTVATLEERPFVIVESPDPGTGGCVPNTVPCRRQSNHTFSSGDVAPYTKLCCKGFCIDILKKLARVVKFSYDLYLVTNGKHGKRVRGVWNGMIGEVYYKRADMAIGSLTINEERSEIVDFSVPFVETGISVMVARSNGTVSPSAFLEPYSPAVWVMMFVMCLTVVAITVFMFEYFSPVSYNQNLTRGKKSGGPAFTIGKSVWLLWALVFNNSVPIENPRGTTSKIMVLVWAFFAVIFLASYTANLAAFMIQEQYIDTVSGLSDKKFQRPQDQYPPFRFGTVPNGSTERNIRSNYRDMHTHMVKFNQRSVEDALTSLKMGKLDAFIYDAAVLNYMAGKDEGCKLVTIGSGKVFATTGYGIAMQKDSHWKRAIDLALLQFLGDGETQKLETVWLSGICQNEKNEVMSSKLDIDNMAGVFYMLLVAMGLALLVFAWEHLVYWKLRHSVPNSSQLDFLLAFSRGIYSCFSGVQSLASPPRQASPDLTASSAQASVLKMLQAARDMVTTAGVSSSLDRATRTIENWGGGRRAPPPSPCPTPRSGPSPCLPTPDPPPEPSPTGWGPPDGGRAALVRRAPQPPGRPPTPGPPLSDVSRVSRRPAWEARWPVRTGHCGRHLSASERPLSPARCHYSSFPRADRSGRPFLPLFPEPPELEDLPLLGPEQLARREALLHAAWARGSRPRHASLPSSVAEAFARPSSLPAGCTGPACARPDGHSACRRLAQAQSMCLPIYREACQEGEQAGAPAWQHRQHVCLHAHAHLPFCWGAVCPHLPPCASHGSWLSGAWGPLGHRGRTLGLGTGYRDSGGLDEISSVARGTQGFPGPCTWRRISSLESEV

SEQ ID NO: 9 - DNA, 4299 bp, Homo sapiens
ggacgccgggttccggtcctggcccccccgccatccccccaacagaacagggtcatgaaaagaggccgcccggcggggcccgcaggcgatgcgcggcgccggtggcccccgcggccctcggggccccgctaagatgctgctgctgctggcgctggcctgcgccagcccgttcccggaggaggcgccggggccgggcggggccggtgggcccggcggcggcctcggcggggcgcggccgctcaacgtggcgctcgtgttctcggggcccgcgtacgcggccgaggcggcacgcctgggcccggccgtggcggcggcggtgcgcagcccgggcctagacgtgcggcccgtggcgctggtgctcaacggctcggacccgcgcagcctcgtgctgcagctctgcgacctgctgtcggggttgcgcgtgcacggcgtggtcttcgaagacgactcgcgcgcgcccgccgtcgcgcccatcctcgacttcctgtcggcgcagacctcgctgcccatcgtggccgtgcacggcggcgccgcgctcgtgctcacgcccaaggagaagggctccaccttcctgcagctgggctcttccaccgagcaacagcttcaggtcatctttgaggtgctggaggagtatgactggacgtcctttgtagccgtgaccactcgtgcccctggccaccgggccttcctgtcctacattgaggtgctgactgacggtagtctggtgggctgggagcaccgcggagcgctgacgctggaccctggggcgggcgaggccgtgctcagtgcccagctccgcagtgtcagcgcgcagatccgcctgctcttctgcgcccgagaggaggccgagcccgtgttccgcgcagctgaggaggctggcctcactggatctggctacgtctggttcatggtggggccccagctggctggaggcgggggctctggggcccctggtgagccccctcttctgccaggaggcgcccccctgcctgccgggctgtttgcagtgcgctcggctggctggcgggatgacctggctcggcgagtggcagctggcgtggccgtagtggccagaggtgcccaggccctgctgcgtgattatggtttccttcctgagctcggccacgactgtcgcgcccagaaccgcacccaccgcggcgagagtctgcataggtacttcatgaacatcacgtgggataaccgggattactccttcaatgaggacggcttcctagtgaacccctccctggtggtcatctccctcaccagagacaggacgtgggaggtggtgggcagctgggagcagcagacgctccgcctcaagtacccgctgtggtcccgctatggtcgcttcctgcagccagtggacgacacgcagcacctcacggtggccacgctggaggaaaggccgtttgtcatcgtggagcctgcagaccctatcagcggcacctgcatccgagactccgtcccctgccggagccagctcaaccgaacccacagccctccaccggatgccccccgcccggaaaagcgctgctgcaagggtttctgcatcgacattctgaagcggctggcgcataccatcggcttcagctacgacctctacctggtcaccaatggcaagcacggaaagaagatcgatggcgtctggaacggcatgatcggggaggtgttctaccagcgcgcagacatggccatcggctccctcaccatcaacgaggagcgctccgagatcgtggacttctccgtccccttcgtggagaccggcatcagcgtcatggtggcgcgcagcaatggcacggtgtccccctcggccttcctcgagccctacagccccgccgtgtgggtgatgatgttcgtcatgtgcctcactgtggtcgccgtcactgttttcatcttcgagtacctcagtcctgttggttacaaccgcagcctggccacgggcaagcgccctggcggttcaaccttcaccattgggaaatccatctggctgctctgggccctggtgttcaataattcggtgcccgtggagaacccccggggaaccaccagcaaaatcatggtgctggtgtgggccttcttcgccgtcatcttcctcgccagctacacagccaacctggccgccttcatgatccaggaggagtacgtggatactgtgtctgggctcagtgaccgcaagttccagaggccccaggagcagtacccgcccctgaagtttgggaccgtgcccaacggctccacggagaagaacatccgcagcaactatcccgacatgcacagctacatggtgcgctacaaccagccccgcgtagaggaagcgctcactcagctcaaggcagggaagctggacgccttcatctacgatgctgcagtgctcaattacatggcccgcaaggacgagggctgcaagcttgtcaccatcggctccggcaaggtcttcgccacgacaggctatggcatcgccctgcacaagggctcccgctggaagcggcccatcgacctggcgttgctgcagttcctgggggatgatgagatcgagatgctggagcggctgtggctctctgggatctgccacaatgacaaaatcgaggtgatgagcagcaagctggacatcgacaacatggcgggcgtcttctacatgctcctggtggccatgggcctgtccctgctggtcttcgcctgggagcacctggtgtactggcgcctgcggcactgcctggggcccacccaccgcatggacttcctgctggccttctccaggggcatgtacagctgctgcagcgctgaggccgccccaccgcccgccaagcccccgccgccgccacagcccctgcccagccccgcgtaccccgcgccggggccggctcccgggcccgcacctttcgtgccccgcgagcgcgcctcagtggaccgctggcgccggaccaagggcgcggggccgccggggggcgcgggcctggccgacggcttccaccgctactacggccccatcgagccgcagggcctaggcctcggcctgggcgaagcgcgcgcggcaccgcggggcgcagccgggcgcccgctgtccccgccggccgctcagcccccgcagaagccgccggcctcctatttcgccatcgtacgcgacaaggagccagccgagccccccgccggcgccttccccggcttcccgtcgccgcccgcgccccccgccgccgcggccaccgccgtcgggccgccactctgccgcttggccttcgaggacgagagcccgccggcgcccgcgcggtggccgcgctcggaccccgagagccaacccctgctggggccaggcgcgggcggcgcggggggcacggggggcgcaggcggaggagccccggccgctccgcccccgtgctgcgccgcgccgcccccgtgcccttacctcgatctcgagccgtcgccgtcggactcggaggactcggagagcctgggcggcgcgtcgctgggcggcctggatccctggtggttcgccgacttcccttacccgtatgccgagcgcctcgggccgccgcccggccgctactggtcggtcgacaagctcgggggctggcgcgccgggagctgggactacctgcccccgcgcagcggtccggccgcctggcactgtcggcactgcgccagcctggagctgctgccgccgccgcgccatctcagctgctcgcacgatggcctggacggcggctggtgggcgccaccgcctccaccctgggccgccgggcccctgccccgacgccgggcccgctgcgggtgcccgcggtcgcacccgcaccgcccgcgggcctcgcaccgcacgcccgccgctgccgcgccccaccaccacaggcaccggcgcgccgctgggggctgggacctcccgccgcccgcgcccacctcgcgctcgctcgaggacctcagctcgtgccctcgcgccgcccctgcgcgcaggcttaccgggccctcccgccacgctcgcaggtgtccgcacgccgcgcactgggggccgccgctgcccacagcttcccaccggagacaccggggcggggacctgggcacccgcaggggctcggcgcacttctctagcctcgagtccgaggtatgacgcggccccgggggccccaccgcccccttggtcagcgcaggccacggcccgagggggcgcccgcagtggacaggacccgcgtgggttgggaaggaaagcagtggaactggccggaccccgcctggagcagcgtcctgcgccccctggttctggaggaaccgcaagccggagaggatttggtccctcaactatcacccagg

SEQ ID NO: 10 - protein, 1336 aa, Homo sapiens
MRGAGGPRGPRGPAKMLLLLALACASPFPEEAPGPGGAGGPGGGLGGARPLNVALVFSGPAYAAEAARLGPAVAAAVRSPGLDVRPVALVLNGSDPRSLVLQLCDLLSGLRVHGVVFEDDSRAPAVAPILDFLSAQTSLPIVAVHGGAALVLTPKEKGSTFLQLGSSTEQQLQVIFEVLEEYDWTSFVAVTTRAPGHRAFLSYIEVLTDGSLVGWEHRGALTLDPGAGEAVLSAQLRSVSAQIRLLFCAREEAEPVFRAAEEAGLTGSGYVWFMVGPQLAGGGGSGAPGEPPLLPGGAPLPAGLFAVRSAGWRDDLARRVAAGVAVVARGAQALLRDYGFLPELGHDCRAQNRTHRGESLHRYFMNITWDNRDYSFNEDGFLVNPSLVVISLTRDRTWEVVGSWEQQTLRLKYPLWSRYGRFLQPVDDTQHLTVATLEERPFVIVEPADPISGTCIRDSVPCRSQLNRTHSPPPDAPRPEKRCCKGFCIDILKRLAHTIGFSYDLYLVTNGKHGKKIDGVWNGMIGEVFYQRADMAIGSLTINEERSEIVDFSVPFVETGISVMVARSNGTVSPSAFLEPYSPAVWVMMFVMCLTVVAVTVFIFEYLSPVGYNRSLATGKRPGGSTFTIGKSIWLLWALVFNNSVPVENPRGTTSKIMVLVWAFFAVIFLASYTANLAAFMIQEEYVDTVSGLSDRKFQRPQEQYPPLKFGTVPNGSTEKNIRSNYPDMHSYMVRYNQPRVEEALTQLKAGKLDAFIYDAAVLNYMARKDEGCKLVTIGSGKVFATTGYGIALHKGSRWKRPIDLALLQFLGDDEIEMLERLWLSGICHNDKIEVMSSKLDIDNMAGVFYMLLVAMGLSLLVFAWEHLVYWRLRHCLGPTHRMDFLLAFSRGMYSCCSAEAAPPPAKPPPPPQPLPSPAYPAPGPAPGPAPFVPRERASVDRWRRTKGAGPPGGAGLADGFHRYYGPIEPQGLGLGLGEARAAPRGAAGRPLSPPAAQPPQKPPASYFAIVRDKEPAEPPAGAFPGFPSPPAPPAAAATAVGPPLCRLAFEDESPPAPARWPRSDPESQPLLGPGAGGAGGTGGAGGGAPAAPPPCCAAPPPCPYLDLEPSPSDSEDSESLGGASLGGLDPWWFADFPYPYAERLGPPPGRYWSVDKLGGWRAGSWDYLPPRSGPAAWHCRHCASLELLPPPRHLSCSHDGLDGGWWAPPPPPWAAGPLPRRRARCGCPRSHPHRPRASHRTPAAAAPHHHRHRRAAGGWDLPPPAPTSRSLEDLSSCPRAAPARRLTGPSRHARRCPHAAHWGPPLPTASHRRHRGGDLGTRRGSAHFSSLESEV

SEQ ID NO: 11 - DNA, 5484 bp, Homo sapiens
aattgtcagcacgggatctgagacttccaaaaaatgaagccggcgacaggactttgggtctgggtgagccttctcgtggcggcggggaccgtccagcccagcgattctcagtcagtgtgtgcaggaacggagaataaactgagctctctctctgacctggaacagcagtaccgagccttgcgcaagtactatgaaaactgtgaggttgtcatgggcaacctggagataaccagcattgagcacaaccgggacctctccttcctgcggtctgttcgagaagtcacaggctacgtgttagtggctcttaatcagtttcgttacctgcctctggagaatttacgcattattcgtgggacaaaactttatgaggatcgatatgccttggcaatatttttaaactacagaaaagatggaaactttggacttcaagaacttggattaaagaacttgacagaaatcctaaatggtggagtctatgtagaccagaacaaattcctttgttatgcagacaccattcattggcaagatattgttcggaacccatggccttccaacttgactcttgtgtcaacaaatggtagttcaggatgtggacgttgccataagtcctgtactggccgttgctggggacccacagaaaatcattgccagactttgacaaggacggtgtgtgcagaacaatgtgacggcagatgctacggaccttacgtcagtgactgctgccatcgagaatgtgctggaggctgctcaggacctaaggacacagactgctttgcctgcatgaatttcaatgacagtggagcatgtgttactcagtgtccccaaacctttgtctacaatccaaccacctttcaactggagcacaatttcaatgcaaagtacacatatggagcattctgtgtcaagaaatgtccacataactttgtggtagattccagttcttgtgtgcgtgcctgccctagttccaagatggaagtagaagaaaatgggattaaaatgtgtaaaccttgcactgacatttgcccaaaagcttgtgatggcattggcacaggatcattgatgtcagctcagactgtggattccagtaacattgacaaattcataaactgtaccaagatcaatgggaatttgatctttctagtcactggtattcatggggacccttacaatgcaattgaagccatagacccagagaaactgaacgtctttcggacagtcagagagataacaggtttcctgaacatacagtcatggccaccaaacatgactgacttcagtgttttttctaacctggtgaccattggtggaagagtactctatagtggcctgtccttgcttatcctcaagcaacagggcatcacctctctacagttccagtccctgaaggaaatcagcgcaggaaacatctatattactgacaacagcaacctgtgttattatcataccattaactggacaacactcttcagcacaatcaaccagagaatagtaatccgggacaacagaaaagctgaaaattgtactgctgaaggaatggtgtgcaaccatctgtgttccagtgatggctgttggggacctgggccagaccaatgtctgtcgtgtcgccgcttcagtagaggaaggatctgcatagagtcttgtaacctctatgatggtgaatttcgggagtttgagaatggctccatctgtgtggagtgtgacccccagtgtgagaagatggaagatggcctcctcacatgccatggaccgggtcctgacaactgtacaaagtgctctcattttaaagatggcccaaactgtgtggaaaaatgtccagatggcttacagggggcaaacagtttcattttcaagtatgctgatccagatcgggagtgccacccatgccatccaaactgcacccaagggtgtaacggtcccactagtcatgactgcatttactacccatggacgggccattccactttaccacaacatgctagaactcccctgattgcagctggagtaattggtgggctcttcattctggtcattgtgggtctgacatttgctgtttatgttagaaggaagagcatcaaaaagaaaagagccttgagaagattcttggaaacagagttggtggaaccattaactcccagtggcacagcacccaatcaagctcaacttcgtattttgaaagaaactgagctgaagagggtaaaagtccttggctcaggtgcttttggaacggtttataaaggtatttgggtacctgaaggagaaactgtgaagattcctgtggctattaagattcttaatgagacaactggtcccaaggcaaatgtggagttcatggatgaagctctgatcatggcaagtatggatcatccacacctagtccggttgctgggtgtgtgtctgagcccaaccatccagctggttactcaacttatgccccatggctgcctgttggagtatgtccacgagcacaaggataacattggatcacaactgctgcttaactggtgtgtccagatagctaagggaatgatgtacctggaagaaagacgactcgttcatcgggatttggcagcccgtaatgtcttagtgaaatctccaaaccatgtgaaaatcacagattttgggctagccagactcttggaaggagatgaaaaagagtacaatgctgatggaggaaagatgccaattaaatggatggctctggagtgtatacattacaggaaattcacccatcagagtgacgtttggagctatggagttactatatgggaactgatgacctttggaggaaaaccctatgatggaattccaacgcgagaaatccctgatttattagagaaaggagaacgtttgcctcagcctcccatctgcactattgacgtttacatggtcatggtcaaatgttggatgattgatgctgacagtagacctaaatttaaggaactggctgctgagttttcaaggatggctcgagaccctcaaagatacctagttattcagggtgatgatcgtatgaagcttcccagtccaaatgacagcaagttctttcagaatctcttggatgaagaggatttggaagatatgatggatgctgaggagtacttggtccctcaggctttcaacatcccacctcccatctatacttccagagcaagaattgactcgaataggagtgaaattggacacagccctcctcctgcctacacccccatgtcaggaaaccagtttgtataccgagatggaggttttgctgctgaacaaggagtgtctgtgccctacagagccccaactagcacaattccagaagctcctgtggcacagggtgctactgctgagatttttgatgactcctgctgtaatggcaccctacgcaagccagtggcaccccatgtccaagaggacagtagcacccagaggtacagtgctgaccccaccgtgtttgccccagaacggagcccacgaggagagctggatgaggaaggttacatgactcctatgcgagacaaacccaaacaagaatacctgaatccagtggaggagaacccttttgtttctcggagaaaaaatggagaccttcaagcattggataatcccgaatatcacaatgcatccaatggtccacccaaggccgaggatgagtatgtgaatgagccactgtacctcaacacctttgccaacaccttgggaaaagctgagtacctgaagaacaacatactgtcaatgccagagaaggccaagaaagcgtttgacaaccctgactactggaaccacagcctgccacctcggagcacccttcagcacccagactacctgcaggagtacagcacaaaatatttttataaacagaatgggcggatccggcctattgtggcagagaatcctgaatacctctctgagttctccctgaagccaggcactgtgctgccgcctccaccttacagacaccggaatactgtggtgtaagctcagttgtggttttttaggtggagagacacacctgctccaatttccccacccccctctctttctctggtggtcttccttctaccccaaggccagtagttttgacacttcccagtggaagatacagagatgcaatgatagttatgtgcttacctaacttgaacattagagggaaagactgaaagagaaagataggaggaaccacaatgtttcttcatttctctgcatgggttggtcaggagaatgaaacagctagagaaggaccagaaaatgtaaggcaatgctgcctactatcaaactagctgtcactttttttctttttctttttctttctttgtttctttcttcctcttctttttttttttttttttaaagcagatggttgaaacacccatgctatctgttcctatctgcaggaactgatgtgtgcatatttagcatccctggaaatcataataaagtttccattagaacaaaagaataacattttctataacatatgatagtgtctgaaattgagaatccagtttctttccccagcagtttctgtcctagcaagtaagaatggccaactcaactttcataatttaaaaatctccattaaagttataactagtaattatgttttcaacactttttggtttttttcattttgttttgctctgaccgattcctttatatttgctcccctatttttggctttaatttctaattgcaaagatgtttacatcaaagcttcttcacagaatttaagcaagaaatattttaatatagtgaaatggccactactttaagtatacaatctttaaaataagaaagggaggctaatatttttcatgctatcaaattatcttcaccctcatcctttacatttttcaacatttttttttctccataaatgacactacttgataggccgttggttgtctgaagagtagaagggaaactaagagacagttctctgtggttcaggaaaactactgatactttcaggggtggcccaatgagggaatccattgaactggaagaaacacactggattgggtatgtctacctggcagatactcagaaatgtagtttgcacttaagctgtaattttatttgttctttttctgaactccattttggattttgaatcaagcaatatggaagcaaccagcaaattaactaatttaagtacatttttaaaaaaagagctaagataaagactgtggaaatgccaaaccaagcaaattaggaaccttgcaacggtatccagggactatgatgagaggccagcacattatcttcatatgtcacctttgctacgcaaggaaatttgttcagttcgtatacttcgtaagaaggaatgcgagtaaggattggcttgaattccatggaatttctagtatgagactatttatatgaagtagaaggtaactctttgcacataaattggtataataaaaagaaaaacacaaacattcaaagcttagggataggtccttgggtcaaaagttgtaaataaatgtgaaacatcttctc

SEQ ID NO: 12 - protein, 1308 aa, Homo sapiens
MKPATGLWVWVSLLVAAGTVQPSDSQSVCAGTENKLSSLSDLEQQYRALRKYYENCEVVMGNLEITSIEHNRDLSFLRSVREVTGYVLVALNQFRYLPLENLRIIRGTKLYEDRYALAIFLNYRKDGNFGLQELGLKNLTEILNGGVYVDQNKFLCYADTIHWQDIVRNPWPSNLTLVSTNGSSGCGRCHKSCTGRCWGPTENHCQTLTRTVCAEQCDGRCYGPYVSDCCHRECAGGCSGPKDTDCFACMNFNDSGACVTQCPQTFVYNPTTFQLEHNFNAKYTYGAFCVKKCPHNFVVDSSSCVRACPSSKMEVEENGIKMCKPCTDICPKACDGIGTGSLMSAQTVDSSNIDKFINCTKINGNLIFLVTGIHGDPYNAIEAIDPEKLNVFRTVREITGFLNIQSWPPNMTDFSVFSNLVTIGGRVLYSGLSLLILKQQGITSLQFQSLKEISAGNIYITDNSNLCYYHTINWTTLFSTINQRIVIRDNRKAENCTAEGMVCNHLCSSDGCWGPGPDQCLSCRRFSRGRICIESCNLYDGEFREFENGSICVECDPQCEKMEDGLLTCHGPGPDNCTKCSHFKDGPNCVEKCPDGLQGANSFIFKYADPDRECHPCHPNCTQGCNGPTSHDCIYYPWTGHSTLPQHARTPLIAAGVIGGLFILVIVGLTFAVYVRRKSIKKKRALRRFLETELVEPLTPSGTAPNQAQLRILKETELKRVKVLGSGAFGTVYKGIWVPEGETVKIPVAIKILNETTGPKANVEFMDEALIMASMDHPHLVRLLGVCLSPTIQLVTQLMPHGCLLEYVHEHKDNIGSQLLLNWCVQIAKGMMYLEERRLVHRDLAARNVLVKSPNHVKITDFGLARLLEGDEKEYNADGGKMPIKWMALECIHYRKFTHQSDVWSYGVTIWELMTFGGKPYDGIPTREIPDLLEKGERLPQPPICTIDVYMVMVKCWMIDADSRPKFKELAAEFSRMARDPQRYLVIQGDDRMKLPSPNDSKFFQNLLDEEDLEDMMDAEEYLVPQAFNIPPPIYTSRARIDSNRSEIGHSPPPAYTPMSGNQFVYRDGGFAAEQGVSVPYRAPTSTIPEAPVAQGATAEIFDDSCCNGTLRKPVAPHVQEDSSTQRYSADPTVFAPERSPRGELDEEGYMTPMRDKPKQEYLNPVEENPFVSRRKNGDLQALDNPEYHNASNGPPKAEDEYVNEPLYLNTFANTLGKAEYLKNNILSMPEKAKKAFDNPDYWNHSLPPRSTLQHPDYLQEYSTKYFYKQNGRIRPIVAENPEYLSEFSLKPGTVLPPPPYRHRNTVV

SEQ ID NO: 13 - DNA, 2650 bp, Homo sapiens
gccgcgctggtggcggcggcgcgtcgttgcagttgcgccatctgtcaggagcggagccggcgaggagggggctgccgcgggcgaggaggaggggtcgccgcgagccgaaggccttcgagacccgcccgccgcccggcggcgagagtagaggcgaggttgttgtgcgagcggcgcgtcctctcccgcccgggcgcgccgcgcttctcccagcgcaccgaggaccgcccgggcgcacacaaagccgccgcccgcgccgcaccgcccggcggccgccgcccgcgccagggagggattcggccgccgggccggggacaccccggcgccgccccctcggtgctctcggaaggcccaccggctcccgggcccgccggggaccccccggagccgcctcggccgcgccggaggagggcggggagaggaccatgtgagtgggctccggagcctcagcgccgcgcagtttttttgaagaagcaggatgctgatctaaacgtggaaaaagaccagtcctgcctctgttgtagaagacatgtggtgtatataaagtttgtgatcgttggcggacattttggaatttagataatgggctgtgtgcaatgtaaggataaagaagcaacaaaactgacggaggagagggacggcagcctgaaccagagctctgggtaccgctatggcacagaccccacccctcagcactaccccagcttcggtgtgacctccatccccaactacaacaacttccacgcagccgggggccaaggactcaccgtctttggaggtgtgaactcttcgtctcatacggggaccttgcgtacgagaggaggaacaggagtgacactctttgtggccctttatgactatgaagcacggacagaagatgacctgagttttcacaaaggagaaaaatttcaaatattgaacagctcggaaggagattggtgggaagcccgctccttgacaactggagagacaggttacattcccagcaattatgtggctccagttgactctatccaggcagaagagtggtactttggaaaacttggccgaaaagatgctgagcgacagctattgtcctttggaaacccaagaggtacctttcttatccgcgagagtgaaaccaccaaaggtgcctattcactttctatccgtgattgggatgatatgaaaggagaccatgtcaaacattataaaattcgcaaacttgacaatggtggatactacattaccacccgggcccagtttgaaacacttcagcagcttgtacaacattactcagagagagctgcaggtctctgctgccgcctagtagttccctgtcacaaagggatgccaaggcttaccgatctgtctgtcaaaaccaaagatgtctgggaaatccctcgagaatccctgcagttgatcaagagactgggaaatgggcagtttggggaagtatggatgggtacctggaatggaaacacaaaagtagccataaagactcttaaaccaggcacaatgtcccccgaatcattccttgaggaagcgcagatcatgaagaagctgaagcacgacaagctggtccagctctatgcagtggtgtctgaggagcccatctacatcgtcaccgagtatatgaacaaaggaagtttactggatttcttaaaagatggagaaggaagagctctgaaattaccaaatcttgtggacatggcagcacaggtggctgcaggaatggcttacatcgagcgcatgaattatatccatagagatctgcgatcagcaaacattctagtggggaatggactcatatgcaagattgctgacttcggattggcccgattgatagaagacaatgagtacacagcaagacaaggtgcaaagttccccatcaagtggacggcccccgaggcagccctgtacgggaggttcacaatcaagtctgacgtgtggtcttttggaatcttactcacagagctggtcaccaaaggaagagtgccatacccaggcatgaacaaccgggaggtgctggagcaggtggagcgaggctacaggatgccctgcccgcaggactgccccatctctctgcatgagctcatgatccactgctggaaaaaggaccctgaagaacgccccacttttgagtacttgcagagcttcctggaagactactttaccgcgacagagccccagtaccaacctggtgaaaacctgtaaggcccgggtctgcggagagaggccttgtcccagaggctgccccacccctccccattagctttcaattccgtagccagctgctccccagcagcggaaccgcccaggatcagattgcatgtgactctgaagctgacgaacttccatggccctcattaatgacacttgtccccaaatccgaacctcctctgtgaagcattcgagacagaaccttgttatttctcagactttggaaaatgcattgtatcgatgttatgtaaaaggccaaacctctgttcagtgtaaatagttactccagtgccaacaatcctagtgctttccttttttaaaaatgcaaatcctatgtgattttaactctgtcttcacctgattcaactaaaaaaaaaaaagtattattttccaaaagtggcctctttgtctaaaacaataaaattttttttcatgttttaacaaaaaccaa

SEQ ID NO: 14 - protein, 537 aa, Homo sapiens
MGCVQCKDKEATKLTEERDGSLNQSSGYRYGTDPTPQHYPSFGVTSIPNYNNFHAAGGQGLTVFGGVNSSSHTGTLRTRGGTGVTLFVALYDYEARTEDDLSFHKGEKFQILNSSEGDWWEARSLTTGETGYIPSNYVAPVDSIQAEEWYFGKLGRKDAERQLLSFGNPRGTFLIRESETTKGAYSLSIRDWDDMKGDHVKHYKIRKLDNGGYYITTRAQFETLQQLVQHYSERAAGLCCRLVVPCHKGMPRLTDLSVKTKDVWEIPRESLQLIKRLGNGQFGEVWMGTWNGNTKVAIKTLKPGTMSPESFLEEAQIMKKLKHDKLVQLYAVVSEEPIYIVTEYMNKGSLLDFLKDGEGRALKLPNLVDMAAQVAAGMAYIERMNYIHRDLRSANILVGNGLICKIADFGLARLIEDNEYTARQGAKFPIKWTAPEAALYGRFTIKSDVWSFGILLTELVTKGRVPYPGMNNREVLEQVERGYRMPCPQDCPISLHELMIHCWKKDPEERPTFEYLQSFLEDYFTATEPQYQPGENL

SEQ ID NO: 15 - DNA, 3416 bp, Homo sapiens
cggtacaggtaagtcggccgggcaggtaggggtgcccgaggagtagtcgctggagtccgcgcctccctgggactgcaatgtgccggtcttagctgctgcctgagaggatgtctggggtgtccgagcccctgagccgagtaaagttgggcacattacgccggcctgaaggccctgcagagcccatggtggtggtaccagtagatgtggaaaaggaggacgtgcgtatcctcaaggtctgcttctatagcaacagcttcaatcctgggaagaacttcaaactggtcaaatgcactgtccagacggagatccgggagatcatcacctccatcctgctgagcgggcggatcgggcccaacatccggttggctgagtgctatgggctgaggctgaagcacatgaagtccgatgagatccactggctgcacccacagatgacggtgggtgaggtgcaggacaagtatgagtgtctgcacgtggaagccgagtggaggtatgaccttcaaatccgctacttgccagaagacttcatggagagcctgaaggaggacaggaccacgctgctctatttttaccaacagctccggaacgactacatgcagcgctacgccagcaaggtcagcgagggcatggccctgcagctgggctgcctggagctcaggcggttcttcaaggatatgccccacaatgcacttgacaagaagtccaacttcgagctcctagaaaaggaagtggggctggacttgtttttcccaaagcagatgcaggagaacttaaagcccaaacagttccggaagatgatccagcagaccttccagcagtacgcctcgctcagggaggaggagtgcgtcatgaagttcttcaacactctcgccggcttcgccaacatcgaccaggagacctaccgctgtgaactcattcaaggatggaacattactgtggacctggtcattggccctaaagggatccgccagctgactagtcaggacgcaaagcccacctgcctggccgagttcaagcagatcaggtccatcaggtgcctcccgctggaggagggccaggcagtacttcagctgggcattgaaggtgccccccaggccttgtccatcaaaacctcatccctagcagaggctgagaacatggctgacctcatagacggctactgccggctgcagggtgagcaccaaggctctctcatcatccatcctaggaaagatggtgagaagcggaacagcctgccccagatccccatgctaaacctggaggcccggcggtcccacctctcagagagctgcagcatagagtcagacatctacgcagagattcccgacgaaaccctgcgaaggcccggaggtccacagtatggcattgcccgtgaagatgtggtcctgaatcgtattcttggggaaggcttttttggggaggtctatgaaggtgtctacacaaatcacaaaggggagaaaatcaatgtagctgtcaagacctgcaagaaagactgcactctggacaacaaggagaagttcatgagcgaggcagtgatcatgaagaacctcgaccacccgcacatcgtgaagctgatcggcatcattgaagaggagcccacctggatcatcatggaattgtatccctatggggagctgggccactacctggagcggaacaagaactccctgaaggtgctcaccctcgtgctgtactcactgcagatatgcaaagccatggcctacctggagagcatcaactgcgtgcacagggacattgctgtccggaacatcctggtggcctcccctgagtgtgtgaagctgggggactttggtctttcccggtacattgaggacgaggactattacaaagcctctgtgactcgtctccccatcaaatggatgtccccagagtccattaacttccgacgcttcacgacagccagtgacgtctggatgttcgccgtgtgcatgtgggagatcctgagctttgggaagcagcccttcttctggctggagaacaaggatgtcatcggggtgctggagaaaggagaccggctgcccaagcctgatctctgtccaccggtcctttataccctcatgacccgctgctgggactacgaccccagtgaccggccccgcttcaccgagctggtgtgcagcctcagtgacgtttatcagatggagaaggacattgccatggagcaagagaggaatgctcgctaccgaacccccaaaatcttggagcccacagccttccaggaacccccacccaagcccagccgacctaagtacagaccccctccgcaaaccaacctcctggctccaaagctgcagttccaggttcctgagggtctgtgtgccagctctcctacgctcaccagccctatggagtatccatctcccgttaactcactgcacaccccacctctccaccggcacaatgtcttcaaacgccacagcatgcgggaggaggacttcatccaacccagcagccgagaagaggcccagcagctgtgggaggctgaaaaggtcaaaatgcggcaaatcctggacaaacagcagaagcagatggtggaggactaccagtggctcaggcaggaggagaagtccctggaccccatggtttatatgaatgataagtccccattgacgccagagaaggaggtcggctacctggagttcacagggcccccacagaagcccccgaggctgggcgcacagtccatccagcccacagctaacctggaccggaccgatgacctggtgtacctcaatgtcatggagctggtgcgggccgtgctggagctcaagaatgagctctgtcagctgccccccgagggctacgtggtggtggtgaagaatgtggggctgaccctgcggaagctcatcgggagcgtggatgatctcctgccttccttgccgtcatcttcacggacagagatcgagggcacccagaaactgctcaacaaagacctggcagagctcatcaacaagatgcggctggcgcagcagaacgccgtgacctccctgagtgaggagtgcaagaggcagatgctgacggcttcacacaccctggctgtggacgccaagaacctgctcgacgctgtggaccaggccaaggttctggccaatctggcccacccacctgcagagtgacggagggtgggggccacctgcctgcgtcttccgcccctgcctgccatgtacctcccctgccttgctgttggtcatgtgggtcttccagggagaaggccaaggggagtcaccttcccttgccactttgcacgacgccctctccccacccctacccctggctgtactgctcaggctgcagctggacagaggggactctgggctatggacacagggtgacggtgacaaagatggctcagagggggactgctgctgcctggccactgctccctaagccagcct

SEQ ID NO: 16 - protein, 1009 aa, Homo sapiens
MSGVSEPLSRVKLGTLRRPEGPAEPMVVVPVDVEKEDVRILKVCFYSNSFNPGKNFKLVKCTVQTEIREIITSILLSGRIGPNIRLAECYGLRLKHMKSDEIHWLHPQMTVGEVQDKYECLHVEAEWRYDLQIRYLPEDFMESLKEDRTTLLYFYQQLRNDYMQRYASKVSEGMALQLGCLELRRFFKDMPHNALDKKSNFELLEKEVGLDLFFPKQMQENLKPKQFRKMIQQTFQQYASLREEECVMKFFNTLAGFANIDQETYRCELIQGWNITVDLVIGPKGIRQLTSQDAKPTCLAEFKQIRSIRCLPLEEGQAVLQLGIEGAPQALSIKTSSLAEAENMADLIDGYCRLQGEHQGSLIIHPRKDGEKRNSLPQIPMLNLEARRSHLSESCSIESDIYAEIPDETLRRPGGPQYGIAREDVVLNRILGEGFFGEVYEGVYTNHKGEKINVAVKTCKKDCTLDNKEKFMSEAVIMKNLDHPHIVKLIGIIEEEPTWIIMELYPYGELGHYLERNKNSLKVLTLVLYSLQICKAMAYLESINCVHRDIAVRNILVASPECVKLGDFGLSRYIEDEDYYKASVTRLPIKWMSPESINFRRFTTASDVWMFAVCMWEILSFGKQPFFWLENKDVIGVLEKGDRLPKPDLCPPVLYTLMTRCWDYDPSDRPRFTELVCSLSDVYQMEKDIAMEQERNARYRTPKILEPTAFQEPPPKPSRPKYRPPPQTNLLAPKLQFQVPEGLCASSPTLTSPMEYPSPVNSLHTPPLHRHNVFKRHSMREEDFIQPSSREEAQQLWEAEKVKMRQILDKQQKQMVEDYQWLRQEEKSLDPMVYMNDKSPLTPEKEVGYLEFTGPPQKPPRLGAQSIQPTANLDRTDDLVYLNVMELVRAVLELKNELCQLPPEGYVVVVKNVGLTLRKLIGSVDDLLPSLPSSSRTEIEGTQKLLNKDLAELINKMRLAQQNAVTSLSEECKRQMLTASHTLAVDAKNLLDAVDQAKVLANLAHPPAE

SEQ ID NO: 17 - protein, 8 aa, Artificial sequence
  Synthetic peptide
EIISEVQR

SEQ ID NO: 18 - protein, 13 aa, Artificial sequence
  Synthetic peptide
AFNGSSNGHVYEK